SEQUENCE LISTING (32 sequences)

SEQ ID NO: 1 - DNA, 5622 bp, mammalian
  CDS (1)..(5619)
atgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagaaaaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagaccgtgctggaattcatggacctccacttataattgaaggaagtgaatattggtttgttgctgatcttcacctaaactatgaagaagccgtcctgtactgtgccagcaatcacagctttcttgcgactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactattcaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataattctcttatgtggtttgataagaccccactgtcatatacacattggagagcaggaagaccaactataaaaaatgagaagtttttggctggtttaagtactgacggcttctgggatattcaaacctttaaagttattgaagaagcagtttattttcaccagcacagcattcttgcttgtaaaattgaaatggttgactacaaagaagaacataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaatctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgaccagtcttggagttggttagatggatcagaagtgacatttgtcaaatgggaaaataaaagtaagagtggtgttggaagatgtagcatgttgatagcttcaaatgaaacttggaaaaaagttgaatgtgaacatggttttggaagagttgtctgcaaagtgcctctggactgtccttcatctacttggattcagttccaagacagttgttacatttttctccaagaagccatcaaagtagaaagcatagaggatgtcagaaatcagtgtactgaccatggagcggacatgataagcatacataatgaagaagaaaatgcttttatactggatactttgaaaaagcaatggaaaggcccagatgatatcctactaggcatgttttatgacacagatgatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctatcccatacaaaaggaaatatttatcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaa

SEQ ID NO: 2 - protein, 1873 aa, mammalian [sequence omitted]

SEQ ID NO: 3 - DNA, 5622 bp, mammalian
tactcctgtccgacccgctggggagcggcgggccgccccgaggagtacgacgagaagaccaagaagctagagcgcctcgggagaccggcgcgtcgattactggggaagtggtagcaggtacctttatgcccgttcacgtagttcggtcacataccgacctatcatcgtctgctgacactactttgactcctgttcaataccttcacccacagggtcgtagccgagaaagtaaacgtgagggttttcacggaaccggagctataatggtttagccatttactcgactcttacaagtcgacactgaggtcacggtacgacaccacctttacactcgtggtgagagacatgcctcgacgggccatggccgaccgagacttcctacctgtaccgtgtcgttagagtttacgtagactacagaccttctttcctccgagtctcctttcggaaacactggtcggaatagtactctagatatggtctctacccttgagaataccctctggaacacttaaaggtaagaattaactaccctggaccgtagtactaacgtaagaactacttctagtatcacccggtaccacacggtggtggaatttaatacttatactggctttcaccccgtagacgaatttcggacttttgccaacacttctattaacccttttcttgctcgtcaaaccttcaacgatggttaaattatgagtctgccgagaaagaacctttcttcgaatacaaagtacagtcttagttcctcgactaaatgactcgtagttgtcacgacgacttaattgaatggaatttctttttcttccgtaacgattctaaaagacctaaccaaatttagtcgatatgagacgatctccgacccttaccagtctggtgtttggtaatttgaaagagttgaccctaggtctgtccgggtcacgtggatgatatccaccgaggtcgacacgttcttacctacgactcagaccagacaccgtctcgaaaaggacacttcgagttgacgggatacagacgtcctttggtaatttattatgtcacctcaattgtctacagacctgtatgagtctatgggcgacactacgtccgaccgacggtttattacctaaaacgatagacgaccatttactttcattaaggaccctattccgtgtacgctttacgtttcggaagtcatcactggattagtcgtaagtaagagatcgtctacacctccaccaacagtgttttgaggtattactcctatagtttcttcttcacacctatccggaattcttgtatttgtatggttgaaataaagtcaccagtctaccatgacttcaatgagattgtataaccctactcttactcggtttacaagggatgttattctgcgggttgacacaaaggatgaatcctctcgatccagtcacctttcaggttagtacactcctctttgattttatacatacgttctctttccctctttttgacttactgcgttcaagactattctacacaggaggtctactcccgaccttctctgtacctctttggacaatgttctaaatactcctactccagggaaaaccttgtttgacgttagactgatagtgatcgtctaaactcgttcttatggatttactaaactactttttcatactatttagagattcttttatgaagacctgaccggactctctacatctaagaacacctctcatattgacccgttgacaaccaccttcttccgcccgacattggaaaaggttgaccttaaaagaactcggtcgaaggggcccgccgacgcaccgatacagatgacctttcagacaacctttcaccctccacttcctgacgtcttcgaagtttcgtgaaagttaaacgttcttttactcacctggggaacccggacttcttcgtaggggattcggactactggggacaggacttccgaccgtctcaaaggggcgttcagaaagaacaatattccataaggtacgtctttcttaacattctttctccttgacccttcttcgacttgctaagacggttcgggaacctcgtgtggaaagatcgaagtcggtacacctactttatttccttaaagaagtgaaaaattgcctggtcaagtcaccggtcgtaaccgacacctaaccaaacttattttcctcgggtctaaatgttcctaggaccgttacctcactagcatgtggtcacagatgataatagtacggtttactcaaagtcgtcctaatactgtagtctctgacacgacgacagttccataaagtatccggtaccgcttctccgaccgtaaagatactactatctcttaaataaataaactccggaaaacgaacactatgttttgaacttacccacacggtttaaggttttccggcatgaggtttttgtggtctgaccatgttaggtctggcacgaccttaagtacctggaggtgaatattaacttccttcacttataaccaaacaacgactagaagtggatttgatacttcttcggcaggacatgacacggtcgttagtgtcgaaagaacgctgatattgtagaaaacaccctgattttcggtagtttttgttttatcgtttatatagaccactacctgtcttcaccacctattcttaatcgctcaccggttatctactagtaaaatgtatgagtgctataggtaccgtggcgaaaggacactgtaaacctctccttacgaacatgtacagacggttctgaaccgaatagctgaatccatttggttgtctgacatcatggttcaacgggaagtagacactttttatattacaaagaagcaatctctttatgtcgggtctaagacgtcgatttcacgttacaagactcgttacctaaggaaaagtcttattcacaaaagatttctagtttgggcacagagagtgtaaaagagttcgttcgctatggacagtgaggataccaccgtgggaaggaagtcacaactcggtctaacttgttctgaaataatgtaggaacgaaggcctataccttcgatgaaatacctaaccaaacgcgacctgacggatacttttctatttgtttacctgtctattgtctctcgactgcatgtcattgaaagtgggtaataaccaatcaccctccgactcttatggtcttttaaaaaaactccttctcagagcgatggtgacacgggattatgagttggaggtttttagtggcaaatgaccctgcaccttaaaatgtaggacgtcacttgcggtgaaacacagagagacagtctttataagtcttcaattttcgtctgtctgcaacgtcttacgaagtctttgacatttcatagatttattagacatgttttattagggtttctgagactgaaccgtgtcacgattttccctcacagacttttcattgtacgtcgaccactcgtagtgcctgggaatggtcgtccgtaaggagtcacacgtccgcgaggaagtgttgagaaggaatacctagcctgagaagtcagttctactacttgagttgaaaccaaccagtctaccctttgcagaagtaaaatcagcgacccgactttgattacccgttgagcttctgacacatcataatctgtgactacctaagaccttttgtcaactaacgttactgttagttggtccacgataaacgatgataagtcctttactctgactttttctccagtttggtcaactgtcacaatttacaggtagaggacaagatttatgaggcacctatggtaaagtcttgacaacgatgttaaagtattattgtttcttatccgtataccgttgttgtgtcctacttcaagtatgatttacggtctttgacttaggttttagtgtataagactcataagctctacttttcctcttattgaaacaagaactcgttgacgacatgaagttaatataccgaagtacccagtacaatccttattgaatatctttattaagagaatacaccaaactattctggggtgacagtatatgtgtaacctctcgtccttctggttgatattttttactcttcaaaaaccgaccaaattcatgactgccgaagaccctataagtttggaaatttcaataacttcttcgtcaaataaaagtggtcgtgtcgtaagaacgaacattttaactttaccaactgatgtttcttcttgtattatgatgtgacggtgtcaaatacggtatacttctaccataaatgtcacaataagtttttttccattgtaccatacttcgtaatttgtacacaagagtttcacctccagtgaaccgttcgcaagtgttggttttaccggtcgagaaagaccttctataacattttgcactacctaaaggtgatacccaacccgagagttcagtactaccttcacttagttcaaaacttaccagactaccatcatgtaaactgatatagggtacctttccggtttgtagaggacctttaacacaagagaacctaggttttccttgaacctttgtactttttacgttgagacaattcctaccacgataaacaatatttggatgttttagatttttcgacagggcagaatgtataagtagttctacaggtcgtcgttttctcttacccagtgccacctaggtcatgttcccagtgacaatgttcagactagtccgtaacgtgtcaaaaagtctccggttttttaacacaagttttgtactagtgagacgttgatagcaaaggtattttctacttctactcttatttaaacactcgtctgactactcccttttattattgtaatggtactctcaaaccgaacctaatagagttgtaagacaactggtcagaacctcaaccaatctacctagtcttcactgtaaacagtttacccttttattttcattctcaccacaaccttctacatcgtacaactatcgaagtttactttgaaccttttttcaacttacacttgtaccaaaaccttctcaacagacgtttcacggagacctgacaggaagtagatgaacctaagtcaaggttctgtcaacaatgtaaaaagaggttcttcggtagtttcatctttcgtatctcctacagtctttagtcacatgactggtacctcgcctgtactattcgtatgtattacttcttcttttacgaaaatatgacctatgaaactttttcgttacctttccgggtctactataggatgatccgtacaaaatactgtgtctactacgctcaaagttcaccaaactattaagtttatactgtaaactattcacctgtctggttctactactactcctaaatcaactgtggacacgaaaagacgtgtagttctgtccacttaccttttttcctttaacacttcaaagaagacaccttccttgtgatacgttttgtcgatagggtatgttttcctttataaatagtctattggtgtaaaattatagtcgtaaccactaacgatcgtgccattaaaactgtcaaaaccctcgttagtaaaccaaggacatgttttttgtaagactaagagcaaagtggtgtcaaaaaagttggcgtggggttagtggaatattacttctgacacaaaaccatcaacctcttcttttacttataggacaagttaaactgatt

SEQ ID NO: 4 - DNA, 3740 bp, mammalian
  CDS (45)..(740)
ctctccggccgcgcagccgctgccgcccacccgcacccgccgtcatgctccgggccgcgctgcccgcgctcctgctgccgttgctgggcctcgccgctgctgccgtcgcggactgtccttcatctacttggattcagttccaagacagttgttacatttttctccaagaagccatcaaagtagaaagcatagaggatgtcagaaatcagtgtactgaccatggagcggacatgataagcatacataatgaagaagaaaatgcttttatactggatactttgaaaaagcaatggaaaggcccagatgatatcctactaggcatgttttatgacacagatgatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctatcccatacaaaaggaaatatttatcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaagtttttggtaatcttgcactaagacatcaacaaatgccctggcagagataacttgggaaagattttaatataaaacttgacattggatattagagctttaatggtattccttattccagtaacatttttatgtactcatctgctgtgaaaagtctttaggttcattaaaaaaacaggttttagaaatgatcttagatctaatatagtgattttaagcatcccgtcaaaggcagaatctgtcacttgaatgaaggaaagcttaaagcccaagcagataaaaataaaagcccagcctatttgtcttgcctgctgtatcttccctatttagttgacccactttagtttatatgtttattagtaaacatgaaatggggaataagtgattttaagtacatcccatacatttaaatatctttgataattgttatttttttggcagataattcctctagaatgtgtatctttttatgatttagatgaagaaaattttacaacttttaacaccccacaccaattttagtttcattacttttacacacaccattttatcacaaatgactcaagttttaatgaatgtttataaattatttgaaacaaaatatgatcgctgtgtccaggatggcatagagaaagctggcaattaggttaacacttacatattatagtgcccctttaaggatttctctcttgccaccataccttttgtactttcccctatacaagatgtatctcattctcctcaagcatttataaatttttccttcaatgacatgaaaactgtgcaagcaaaaaccgaagaaaaacacttaagtacaactgtagtgacagtgatcaaagttttcagtgcatttattgtacattttaagaaaaaggtgaaaatcatttggggagtaaaaaaatgaaaaagctgaaacgagtaattttcctcaccatcaataaaccaaaaacaggaaagataaagaatgtataaatttcacgtaaattagtcacgtatcacttatcaatggggatacgttctaagaaatgcatagttagggaatcttgtgtgaaaatcagcttgtatttacacaaacccagatggtagagcctattttgtcccaaacctacacagcatgttactgtgctgaatactgcagacaattgtaacacaatatttgtgtatctaaatatagaaaaggtacagtaaaaatatggtctactaaggaaacactgttctatatgtggtccattactgactgaagtatactgtctagaagtctgaggctcaaagaaaagtaatccctcttctgaatccacaccccatcaattatcttactttcttctggggagatagatagatatactatctcactagcttgactaatggcaacaaagttccagcttgtgtagtctctttttattgaccacatgaatcgaaaacactcatcacaattaatggcactatcattaatgagacatgagtaactaaaaagtgatagaaaactattacagtgcggctacatggtactgaaaatgcaggcattacaccagctgttacacaagcacaagcatgctctgtaagagctttacatttctgagattttgtatagtgattgagatgtctattttattattgatagactattactaatgtcaatattgaacactaccctggaattcctgcctggttttcctacccaaattgtaccactccttgaagaactacaggcacagtaaaaaaatatggcgtattatgtgaactaaaagagttctaaaggagttcttaaaggagtggtagaatttgggtaggaaagtgattaagtccaacttaaaaccaacagtctcaaacgtctacaactacaatgtccaatgagccactagccacatgaggctatttaagtaaatttagtttaaaatccagttttcgaattacattagccacattgtcaagtgttcaaatcacaggtggttagtggctactgtactgggcaacatacattatagaacattttcattataggaagttttattgggcagtgctgctcttaaatcctaccttccactcaactcccatacaactttcttttgtacattttgatactttctacctaatggcagctcttccaaaatagctgctttaaactctgatttaattttcaatatttggtttcatttttcaacaggccaagaggcctctggtaatgaagtgctatatatatatatatatgacggagtctcactgtgctgcccaggctacagtgcagtggctcgatcttggctctctccaatctccgccttgcaggttttcaagcaattctcctgcctcagcctccttagtagctgggaccacagacatctgtcaccacacccagctaactttttgtatttttggtagagacggggtttcgccatattgactgggctggtctcaaactcctgacctcaagtgatccacccaccttggtctcccaaagtgctgggattacatgcgtgagccaccacacttggcctacattttttctttatataccagaacatctataacaggcaccttatctactcattagtgaagagataattggattacacaggcaggcttgtttactacatccagaatgtagaaactgctttcttcaacatcttggttctagctagtaataacaatataattctttggcagatattcagaataacattttaaactacattttcttagaaaattgcattcttgtagtgagcagtgtatggtctcttttgttcagaatttaaaactgataaccaatgaaagccttttctcttattcctctaccgtcatttacatgataatctgaagctaatatgacaatatttaaatactaagtggtactagggaactacaagaatactgtaaagcttaagccattgttatcactgtcatttagcatttaataacaaaactatacagaattatgtgcataccaatgaatgttttgtaccatctagttaaattttttaaataaagttttatgggttaagcag

SEQ ID NO: 5 - protein, 232 aa, mammalian
MLRAALPALLLPLLGLAAAAVADCPSSTWIQFQDSCYIFLQEAIKVESIEDVRNQCTDHGADMISIHNEEENAFILDTLKKQWKGPDDILLGMFYDTDDASFKWFDNSNMTFDKWTDQDDDEDLVDTCAFLHIKTGEWKKGNCEVSSVEGTLCKTAIPYKRKYLSDNHILISALVIASTVILTVLGAIIWFLYKKHSDSRFTTVFSTAPQSPYNEDCVLVVGEENEYPVQFD

SEQ ID NO: 6 - DNA, 3740 bp, mammalian
gagaggccggcgcgtcggcgacggcgggtgggcgtgggcggcagtacgaggcccggcgcgacgggcgcgaggacgacggcaacgacccggagcggcgacgacggcagcgcctgacaggaagtagatgaacctaagtcaaggttctgtcaacaatgtaaaaagaggttcttcggtagtttcatctttcgtatctcctacagtctttagtcacatgactggtacctcgcctgtactattcgtatgtattacttcttcttttacgaaaatatgacctatgaaactttttcgttacctttccgggtctactataggatgatccgtacaaaatactgtgtctactacgctcaaagttcaccaaactattaagtttatactgtaaactattcacctgtctggttctactactactcctaaatcaactgtggacacgaaaagacgtgtagttctgtccacttaccttttttcctttaacacttcaaagaagacaccttccttgtgatacgttttgtcgatagggtatgttttcctttataaatagtctattggtgtaaaattatagtcgtaaccactaacgatcgtgccattaaaactgtcaaaaccctcgttagtaaaccaaggacatgttttttgtaagactaagagcaaagtggtgtcaaaaaagttggcgtggggttagtggaatattacttctgacacaaaaccatcaacctcttcttttacttataggacaagttaaactgattcaaaaaccattagaacgtgattctgtagttgtttacgggaccgtctctattgaaccctttctaaaattatattttgaactgtaacctataatctcgaaattaccataaggaataaggtcattgtaaaaatacatgagtagacgacacttttcagaaatccaagtaatttttttgtccaaaatctttactagaatctagattatatcactaaaattcgtagggcagtttccgtcttagacagtgaacttacttcctttcgaatttcgggttcgtctatttttattttcgggtcggataaacagaacggacgacatagaagggataaatcaactgggtgaaatcaaatatacaaataatcatttgtactttaccccttattcactaaaattcatgtagggtatgtaaatttatagaaactattaacaataaaaaaaccgtctattaaggagatcttacacatagaaaaatactaaatctacttcttttaaaatgttgaaaattgtggggtgtggttaaaatcaaagtaatgaaaatgtgtgtggtaaaatagtgtttactgagttcaaaattacttacaaatatttaataaactttgttttatactagcgacacaggtcctaccgtatctctttcgaccgttaatccaattgtgaatgtataatatcacggggaaattcctaaagagagaacggtggtatggaaaacatgaaaggggatatgttctacatagagtaagaggagttcgtaaatatttaaaaaggaagttactgtacttttgacacgttcgtttttggcttctttttgtgaattcatgttgacatcactgtcactagtttcaaaagtcacgtaaataacatgtaaaattctttttccacttttagtaaacccctcatttttttactttttcgactttgctcattaaaaggagtggtagttatttggtttttgtcctttctatttcttacatatttaaagtgcatttaatcagtgcatagtgaatagttacccctatgcaagattctttacgtatcaatcccttagaacacacttttagtcgaacataaatgtgtttgggtctaccatctcggataaaacagggtttggatgtgtcgtacaatgacacgacttatgacgtctgttaacattgtgttataaacacatagatttatatcttttccatgtcatttttataccagatgattcctttgtgacaagatatacaccaggtaatgactgacttcatatgacagatcttcagactccgagtttcttttcattagggagaagacttaggtgtggggtagttaatagaatgaaagaagacccctctatctatctatatgatagagtgatcgaactgattaccgttgtttcaaggtcgaacacatcagagaaaaataactggtgtacttagcttttgtgagtagtgttaattaccgtgatagtaattactctgtactcattgatttttcactatcttttgataatgtcacgccgatgtaccatgacttttacgtccgtaatgtggtcgacaatgtgttcgtgttcgtacgagacattctcgaaatgtaaagactctaaaacatatcactaactctacagataaaataataactatctgataatgattacagttataacttgtgatgggaccttaaggacggaccaaaaggatgggtttaacatggtgaggaacttcttgatgtccgtgtcatttttttataccgcataatacacttgattttctcaagatttcctcaagaatttcctcaccatcttaaacccatcctttcactaattcaggttgaattttggttgtcagagtttgcagatgttgatgttacaggttactcggtgatcggtgtactccgataaattcatttaaatcaaattttaggtcaaaagcttaatgtaatcggtgtaacagttcacaagtttagtgtccaccaatcaccgatgacatgacccgttgtatgtaatatcttgtaaaagtaatatccttcaaaataacccgtcacgacgagaatttaggatggaaggtgagttgagggtatgttgaaagaaaacatgtaaaactatgaaagatggattaccgtcgagaaggttttatcgacgaaatttgagactaaattaaaagttataaaccaaagtaaaaagttgtccggttctccggagaccattacttcacgatatatatatatatatactgcctcagagtgacacgacgggtccgatgtcacgtcaccgagctagaaccgagagaggttagaggcggaacgtccaaaagttcgttaagaggacggagtcggaggaatcatcgaccctggtgtctgtagacagtggtgtgggtcgattgaaaaacataaaaaccatctctgccccaaagcggtataactgacccgaccagagtttgaggactggagttcactaggtgggtggaaccagagggtttcacgaccctaatgtacgcactcggtggtgtgaaccggatgtaaaaaagaaatatatggtcttgtagatattgtccgtggaatagatgagtaatcacttctctattaacctaatgtgtccgtccgaacaaatgatgtaggtcttacatctttgacgaaagaagttgtagaaccaagatcgatcattattgttatattaagaaaccgtctataagtcttattgtaaaatttgatgtaaaagaatcttttaacgtaagaacatcactcgtcacataccagagaaaacaagtcttaaattttgactattggttactttcggaaaagagaataaggagatggcagtaaatgtactattagacttcgattatactgttataaatttatgattcaccatgatcccttgatgttcttatgacatttcgaattcggtaacaatagtgacagtaaatcgtaaattattgttttgatatgtcttaatacacgtatggttacttacaaaacatggtagatcaatttaaaaaatttatttcaaaatacccaattcgtc

SEQ ID NO: 7 - DNA, 1122 bp, mammalian
  CDS (364)..(1047)
aatgccacaagtgctatggattagtcaacagtgctccaccaatgctctgtcctggttcctatccttgcactgatgttatgtaagatgctaacatttggagaagctgccgcaaggattacgggaagttctatttatttttgcaacattttagaaagtctgagattacttcagttcaaatgagaagtttatctttaacgaagagaagttggagtctgcggtgtgtccgcgcttggggatctgagcgtcccagcagtgcgaccctgggctccactcccccgcctcgagtgggaggcgtcgcaactgagctgggagctgcgcacccgacaagcaccgcccccggcccgctctcggcgccgcgcagtcatgccccacgcagcgctgtcctcgctcgtgctgctgagcctcgccactgccatcgtcgccgactgtccttcatctacctgggtccagttccaaggcagctgttatgcttttcttcaagtaaccatcaatgtggaaaacatagaggatgtcagaaaacagtgcactgaccacggggcagacatggtaagcatacacaatgaagaggaaaacgcgtttatactggacactttgcaaaagcgatggaagggtccagatgatctcctgctaggcatgttctatgacactgatgatgcaactttcaagtggtatgatcattcaaatatgacattcgacaagtgggcagatcaagatggtgaggacctagttgatacctgtggttttctgtacaccaagacaggtgaatggagaaaaggggattgtgaaatctcttctgtggagggaacactttgcaaagcagcaatcccatatgacaagaagtatttatcagataaccacattttaatatcgactctggtgatcgctagcacagtaactctggcagttttgggagcgatcatttggttcctctatagaagaaacgcgcgctctggcttcacctctttttcacctgcaccactgtcaccttacagtgatggctgtgccctggtagttgcagaagaagatgaatatgctgttcagctggactaagagtttggtaatatcaggccagcatattgartccattgacaawaatttcctgtgcaaggttttcatataaaa

SEQ ID NO: 8 - protein, 228 aa, mammalian
MPHAALSSLVLLSLATAIVADCPSSTWVQFQGSCYAFLQVTINVENIEDVRKQCTDHGADMVSIHNEEENAFILDTLQKRWKGPDDLLLGMFYDTDDATFKWYDHSNMTFDKWADQDGEDLVDTCGFLYTKTGEWRKGDCEISSVEGTLCKAAIPYDKKYLSDNHILISTLVIASTVTLAVLGAIIWFLYRRNARSGFTSFSPAPLSPYSDGCALVVAEEDEYAVQLD

SEQ ID NO: 9 - DNA, 1122 bp, mammalian
ttacggtgttcacgatacctaatcagttgtcacgaggtggttacgagacaggaccaaggataggaacgtgactacaatacattctacgattgtaaacctcttcgacggcgttcctaatgcccttcaagataaataaaaacgttgtaaaatctttcagactctaatgaagtcaagtttactcttcaaatagaaattgcttctcttcaacctcagacgccacacaggcgcgaacccctagactcgcagggtcgtcacgctgggacccgaggtgagggggcggagctcaccctccgcagcgttgactcgaccctcgacgcgtgggctgttcgtggcgggggccgggcgagagccgcggcgcgtcagtacggggtgcgtcgcgacaggagcgagcacgacgactcggagcggtgacggtagcagcggctgacaggaagtagatggacccaggtcaaggttccgtcgacaatacgaaaagaagttcattggtagttacaccttttgtatctcctacagtcttttgtcacgtgactggtgccccgtctgtaccattcgtatgtgttacttctccttttgcgcaaatatgacctgtgaaacgttttcgctaccttcccaggtctactagaggacgatccgtacaagatactgtgactactacgttgaaagttcaccatactagtaagtttatactgtaagctgttcacccgtctagttctaccactcctggatcaactatggacaccaaaagacatgtggttctgtccacttacctcttttcccctaacactttagagaagacacctcccttgtgaaacgtttcgtcgttagggtatactgttcttcataaatagtctattggtgtaaaattatagctgagaccactagcgatcgtgtcattgagaccgtcaaaaccctcgctagtaaaccaaggagatatcttctttgcgcgcgagaccgaagtggagaaaaagtggacgtggtgacagtggaatgtcactaccgacacgggaccatcaacgtcttcttctacttatacgacaagtcgacctgattctcaaaccattatagtccggtcgtataactyaggtaactgttwttaaaggacacgttccaaaagtatatttt

SEQ ID NO: 10 - DNA, 979 bp, mammalian
  CDS (1)..(672)
  VARIANT (5)..(5): Xaa = Leu
  VARIANT (13)..(13): Xaa = Val, Ala
  VARIANT (142)..(142): Xaa = Thr
cacgaggcctcgctsgtgctgctgagcctagccactgycatcttcgctgactgtccttcgtccatctgggttcagttccaaggcagctgttacacttttcttcaagtaaccatcaatgtggaaaacatagaggatgtcagaaagcagtgtactgatcacggggcagacctggtaagtatacacaatgaagaagaaaacgcatttatactggacactttacaaaagcgatggaaaggcccggatgatcttctgctaggcatgttttatgacactgatgatgcaagtttcaagtggtttgatcagtcaaatatgacattcgacaagtgggcagatgaggatggtgaggacctagttgacacctgtggttttctgtatgccaagacaggtgaatggagaaaaggaaattgtgaaatgtcttctgtgacrggaacactttgcaaaacagcaatcccatatgacaagaagtatttatcagataaccacattttaatatcgactctggtgatcgctagcacagtgactctggcagttttgggagcggtcatttggttcctctatagaaggagcgcacgctctggcttcacctctttctctcctgcaccacaatcaccttacagtgatggctgtgctctggtagttgcggaagaagatgaatactctgttcagctggactgagagtttgggaacatcagacgagcacactgaacaccttgacaagaaataatttcctatgcaagattgtcatgtaaaatttgccacggaaaactgaaccttttatggtattccttattcttctaacaatattttcatgtattcaatgtgacaaaacataaaccttctgattaaaaggaaaaaaagtaggtttcagaaaaggaactagcacagagctaacttacaggttttcttaagtagttttcatttgagtaaatgaaagctacagtacaataaagctggtaaaacgcaaaaaaaaaaaaaaaaa

SEQ ID NO: 11 - protein, 224 aa, mammalian
  VARIANT (5)..(5): Xaa = Leu
  VARIANT (13)..(13): Xaa = Ala or Val
  VARIANT (142)..(142): Xaa = Thr
HEASXVLLSLATXIFADCPSSIWVQFQGSCYTFLQVTINVENIEDVRKQCTDHGADLVSIHNEEENAFILDTLQKRWKGPDDLLLGMFYDTDDASFKWFDQSNMTFDKWADEDGEDLVDTCGFLYAKTGEWRKGNCEMSSVXGTLCKTAIPYDKKYLSDNHILISTLVIASTVTLAVLGAVIWFLYRRSARSGFTSFSPAPQSPYSDGCALVVAEEDEYSVQLD

SEQ ID NO: 12 - DNA, 979 bp, mammalian
gtgctccggagcgascacgacgactcggatcggtgacrgtagaagcgactgacaggaagcaggtagacccaagtcaaggttccgtcgacaatgtgaaaagaagttcattggtagttacaccttttgtatctcctacagtctttcgtcacatgactagtgccccgtctggaccattcatatgtgttacttcttcttttgcgtaaatatgacctgtgaaatgttttcgctacctttccgggcctactagaagacgatccgtacaaaatactgtgactactacgttcaaagttcaccaaactagtcagtttatactgtaagctgttcacccgtctactcctaccactcctggatcaactgtggacaccaaaagacatacggttctgtccacttacctcttttcctttaacactttacagaagacactgyccttgtgaaacgttttgtcgttagggtatactgttcttcataaatagtctattggtgtaaaattatagctgagaccactagcgatcgtgtcactgagaccgtcaaaaccctcgccagtaaaccaaggagatatcttcctcgcgtgcgagaccgaagtggagaaagagaggacgtggtgttagtggaatgtcactaccgacacgagaccatcaacgccttcttctacttatgagacaagtcgacctgactctcaaacccttgtagtctgctcgtgtgacttgtggaactgttctttattaaaggatacgttctaacagtacattttaaacggtgccttttgacttggaaaataccataaggaataagaagattgttataaaagtacataagttacactgttttgtatttggaagactaattttcctttttttcatccaaagtcttttccttgatcgtgtctcgattgaatgtccaaaagaattcatcaaaagtaaactcatttactttcgatgtcatgttatttcgaccattttgcgttttttttttttttttt

SEQ ID NO: 13 - DNA, 483 bp, mammalian
  misc_feature (43)..(43): n = A, C, G or T
gagctagttgacacctgtgcctttttgcacaccaagacaggtngattggaaaaaaggaaactgtgaagtttcttctgtggaaggaaccctttgtaaagcagctatcccatatgaaaagaaatatttatcagataaccgcattttaatatcagctttggtgattgctagcacagtaattctgacagttctgggagcagttgtttggttcttgtacaaaagaagtttggattctggtttcaccacagttttttcagctgcacaccaatcaccttataatgatgactgtgttttagtagttgcagaggaaaacgaatatgatattcaatttaactaagattttggaaatatcagactaagacaaatacctttcagtgattcctctgtaagatttcaatataaaacctgataatgaaaattagtttttatgatatattaccttattccagtaacattcattactcttatgtaaaatcactgatcatg

SEQ ID NO: 14 - DNA, 27 bp, mammalian
  CDS (1)..(27)
aaagtgcctctgggccctgattacaca

SEQ ID NO: 15 - protein, 9 aa, mammalian
KVPLGPDYT

SEQ ID NO: 16 - DNA, 42 bp, mammalian
  CDS (1)..(42)
aaagtgcctctggactgtccttcatctacttggattcagttc

SEQ ID NO: 17 - protein, 14 aa, mammalian
KVPLDCPSSTWIQF

SEQ ID NO: 18 - DNA, 42 bp, mammalian
  CDS (1)..(42)
gctgccgtcgcggactgtccttcatctacttggattcagttc

SEQ ID NO: 19 - protein, 14 aa, mammalian
AAVADCPSSTWIQF

SEQ ID NO: 20 - DNA, 5454 bp, mammalian
  CDS (1)..(5451)
atgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagaaaaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagaccgtgctggaattcatggacctccacttataattgaaggaagtgaatattggtttgttgctgatcttcacctaaactatgaagaagccgtcctgtactgtgccagcaatcacagctttcttgcgactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactattcaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataattctcttatgtggtttgataagaccccactgtcatatacacattggagagcaggaagaccaactataaaaaatgagaagtttttggctggtttaagtactgacggcttctgggatattcaaacctttaaagttattgaagaagcagtttattttcaccagcacagcattcttgcttgtaaaattgaaatggttgactacaaagaagaacataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaatctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgactgtccttcatctacttggattcagttccaagacagttgttacatttttctccaagaagccatcaaagtagaaagcatagaggatgtcagaaatcagtgtactgaccatggagcggacatgataagcatacataatgaagaagaaaatgcttttatactggatactttgaaaaagcaatggaaaggcccagatgatatcctactaggcatgttttatgacacagatgatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctatcccatacaaaaggaaatatttatcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaa

SEQ ID NO: 21 - protein, 1817 aa, mammalian [sequence omitted]

SEQ ID NO: 22 - DNA, 5454 bp, mammalian
tactcctgtccgacccgctggggagcggcgggccgccccgaggagtacgacgagaagaccaagaagctagagcgcctcgggagaccggcgcgtcgattactggggaagtggtagcaggtacctttatgcccgttcacgtagttcggtcacataccgacctatcatcgtctgctgacactactttgactcctgttcaataccttcacccacagggtcgtagccgagaaagtaaacgtgagggttttcacggaaccggagctataatggtttagccatttactcgactcttacaagtcgacactgaggtcacggtacgacaccacctttacactcgtggtgagagacatgcctcgacgggccatggccgaccgagacttcctacctgtaccgtgtcgttagagtttacgtagactacagaccttctttcctccgagtctcctttcggaaacactggtcggaatagtactctagatatggtctctacccttgagaataccctctggaacacttaaaggtaagaattaactaccctggaccgtagtactaacgtaagaactacttctagtatcacccggtaccacacggtggtggaatttaatacttatactggctttcaccccgtagacgaatttcggacttttgccaacacttctattaacccttttcttgctcgtcaaaccttcaacgatggttaaattatgagtctgccgagaaagaacctttcttcgaatacaaagtacagtcttagttcctcgactaaatgactcgtagttgtcacgacgacttaattgaatggaatttctttttcttccgtaacgattctaaaagacctaaccaaatttagtcgatatgagacgatctccgacccttaccagtctggtgtttggtaatttgaaagagttgaccctaggtctgtccgggtcacgtggatgatatccaccgaggtcgacacgttcttacctacgactcagaccagacaccgtctcgaaaaggacacttcgagttgacgggatacagacgtcctttggtaatttattatgtcacctcaattgtctacagacctgtatgagtctatgggcgacactacgtccgaccgacggtttattacctaaaacgatagacgaccatttactttcattaaggaccctattccgtgtacgctttacgtttcggaagtcatcactggattagtcgtaagtaagagatcgtctacacctccaccaacagtgttttgaggtattactcctatagtttcttcttcacacctatccggaattcttgtatttgtatggttgaaataaagtcaccagtctaccatgacttcaatgagattgtataaccctactcttactcggtttacaagggatgttattctgcgggttgacacaaaggatgaatcctctcgatccagtcacctttcaggttagtacactcctctttgattttatacatacgttctctttccctctttttgacttactgcgttcaagactattctacacaggaggtctactcccgaccttctctgtacctctttggacaatgttctaaatactcctactccagggaaaaccttgtttgacgttagactgatagtgatcgtctaaactcgttcttatggatttactaaactactttttcatactatttagagattcttttatgaagacctgaccggactctctacatctaagaacacctctcatattgacccgttgacaaccaccttcttccgcccgacattggaaaaggttgaccttaaaagaactcggtcgaaggggcccgccgacgcaccgatacagatgacctttcagacaacctttcaccctccacttcctgacgtcttcgaagtttcgtgaaagttaaacgttcttttactcacctggggaacccggacttcttcgtaggggattcggactactggggacaggacttccgaccgtctcaaaggggcgttcagaaagaacaatattccataaggtacgtctttcttaacattctttctccttgacccttcttcgacttgctaagacggttcgggaacctcgtgtggaaagatcgaagtcggtacacctactttatttccttaaagaagtgaaaaattgcctggtcaagtcaccggtcgtaaccgacacctaaccaaacttattttcctcgggtctaaatgttcctaggaccgttacctcactagcatgtggtcacagatgataatagtacggtttactcaaagtcgtcctaatactgtagtctctgacacgacgacagttccataaagtatccggtaccgcttctccgaccgtaaagatactactatctcttaaataaataaactccggaaaacgaacactatgttttgaacttacccacacggtttaaggttttccggcatgaggtttttgtggtctgaccatgttaggtctggcacgaccttaagtacctggaggtgaatattaacttccttcacttataaccaaacaacgactagaagtggatttgatacttcttcggcaggacatgacacggtcgttagtgtcgaaagaacgctgatattgtagaaaacaccctgattttcggtagtttttgttttatcgtttatatagaccactacctgtcttcaccacctattcttaatcgctcaccggttatctactagtaaaatgtatgagtgctataggtaccgtggcgaaaggacactgtaaacctctccttacgaacatgtacagacggttctgaaccgaatagctgaatccatttggttgtctgacatcatggttcaacgggaagtagacactttttatattacaaagaagcaatctctttatgtcgggtctaagacgtcgatttcacgttacaagactcgttacctaaggaaaagtcttattcacaaaagatttctagtttgggcacagagagtgtaaaagagttcgttcgctatggacagtgaggataccaccgtgggaaggaagtcacaactcggtctaacttgttctgaaataatgtaggaacgaaggcctataccttcgatgaaatacctaaccaaacgcgacctgacggatacttttctatttgtttacctgtctattgtctctcgactgcatgtcattgaaagtgggtaataaccaatcaccctccgactcttatggtcttttaaaaaaactccttctcagagcgatggtgacacgggattatgagttggaggtttttagtggcaaatgaccctgcaccttaaaatgtaggacgtcacttgcggtgaaacacagagagacagtctttataagtcttcaattttcgtctgtctgcaacgtcttacgaagtctttgacatttcatagatttattagacatgttttattagggtttctgagactgaaccgtgtcacgattttccctcacagacttttcattgtacgtcgaccactcgtagtgcctgggaatggtcgtccgtaaggagtcacacgtccgcgaggaagtgttgagaaggaatacctagcctgagaagtcagttctactacttgagttgaaaccaaccagtctaccctttgcagaagtaaaatcagcgacccgactttgattacccgttgagcttctgacacatcataatctgtgactacctaagaccttttgtcaactaacgttactgttagttggtccacgataaacgatgataagtcctttactctgactttttctccagtttggtcaactgtcacaatttacaggtagaggacaagatttatgaggcacctatggtaaagtcttgacaacgatgttaaagtattattgtttcttatccgtataccgttgttgtgtcctacttcaagtatgatttacggtctttgacttaggttttagtgtataagactcataagctctacttttcctcttattgaaacaagaactcgttgacgacatgaagttaatataccgaagtacccagtacaatccttattgaatatctttattaagagaatacaccaaactattctggggtgacagtatatgtgtaacctctcgtccttctggttgatattttttactcttcaaaaaccgaccaaattcatgactgccgaagaccctataagtttggaaatttcaataacttcttcgtcaaataaaagtggtcgtgtcgtaagaacgaacattttaactttaccaactgatgtttcttcttgtattatgatgtgacggtgtcaaatacggtatacttctaccataaatgtcacaataagtttttttccattgtaccatacttcgtaatttgtacacaagagtttcacctccagtgaaccgttcgcaagtgttggttttaccggtcgagaaagaccttctataacattttgcactacctaaaggtgatacccaacccgagagttcagtactaccttcacttagttcaaaacttaccagactaccatcatgtaaactgatatagggtacctttccggtttgtagaggacctttaacacaagagaacctaggttttccttgaacctttgtactttttacgttgagacaattcctaccacgataaacaatatttggatgttttagatttttcgacagggcagaatgtataagtagttctacaggtcgtcgttttctcttacccagtgccacctaggtcatgttcccagtgacaatgttcagactagtccgtaacgtgtcaaaaagtctccggttttttaacacaagttttgtactagtgagacgttgatagcaaaggtattttctacttctactcttatttaaacactcgtctgactactcccttttattattgtaatggtactctcaaaccgaacctaatagagttgtaagacaactgacaggaagtagatgaacctaagtcaaggttctgtcaacaatgtaaaaagaggttcttcggtagtttcatctttcgtatctcctacagtctttagtcacatgactggtacctcgcctgtactattcgtatgtattacttcttcttttacgaaaatatgacctatgaaactttttcgttacctttccgggtctactataggatgatccgtacaaaatactgtgtctactacgctcaaagttcaccaaactattaagtttatactgtaaactattcacctgtctggttctactactactcctaaatcaactgtggacacgaaaagacgtgtagttctgtccacttaccttttttcctttaacacttcaaagaagacaccttccttgtgatacgttttgtcgatagggtatgttttcctttataaatagtctattggtgtaaaattatagtcgtaaccactaacgatcgtgccattaaaactgtcaaaaccctcgttagtaaaccaaggacatgttttttgtaagactaagagcaaagtggtgtcaaaaaagttggcgtggggttagtggaatattacttctgacacaaaaccatcaacctcttcttttacttataggacaagttaaactgatt

SEQ ID NO: 23 - DNA, 21 bp, synthetic
gaccatggagcggacatgata

SEQ ID NO: 24 - DNA, 21 bp, synthetic
ggctctaccatctgggtttgt

SEQ ID NO: 25 - DNA, 19 bp, synthetic
ccgccatgtcgcgcggcct

SEQ ID NO: 26 - DNA, 24 bp, synthetic
accaaatcagtccgcccatgagaa

SEQ ID NO: 27 - DNA, 24 bp, synthetic
atcatgtccgctccatggtcagta

SEQ ID NO: 28 - DNA, 21 bp, synthetic
tattcagaagttaaaagcaga

SEQ ID NO: 29 - DNA, 21 bp, synthetic
ccaaaaggccgtactccaaaa

SEQ ID NO: 30 - DNA, 21 bp, synthetic
ggaggaaaactgaatgacgca

SEQ ID NO: 31 - DNA, 21 bp, synthetic
gaaaacggttgtgaagataat

SEQ ID NO: 32 - DNA, 32460 bp, human
attttttttaactgggtcatttgttttcacgttgttgagtttagacttgattttatagaactcttttgccaatgacatgaggcttaaacaagcacccttgcttgaccctggctagtgttgtccagaggccctcatgggtgaagtataattgcacagtttatgcatacattgtagcaaacatagcttaaagactcttcatccaggagctggtaacagagaaaagttgtctaagagacatagtccactgaaaggggtctttgaaaaggaggattctggccagtagatccaaagaagaaggctggcaaacgccaccttaaccaactgatccaagttaacatcagtgatgagacatcgacatcttgtccttctcgagcacgtacatcacttctggggaactctttcccaaaaaagcgcaacctgaatccaattagaaggaaatgtcagacaaaatgaaactgagggacattctacaaaacgactggccagtactttttaaatgtggtttgatcgtgaaagaaaaagaatgacagagaaccgtcctagattaaagggagactaaggagacacgacatgcaatgtatgatcctggattgaatcttggaacagaaaaggacccttagaggggcaattaatgaaatgtggtaagtgctgtagattagctaatcgcattatcccagtgcgagtgccctgtgtttgatcactgcactggcgttatatacaatgtcaatatttagagaagctggctgaagggttacggaaatctcttgtgctgtttttgcaacattttcgaaagtcctaaattatttcaattcaaatgaaaagtttaaaaaacaaaaattagaagttccagaaggccgcgcgccagccgctcctgcgggacgggacacccgggttctcctggtgggagcccccagtgccgttcacgttccgcccgggggggggattaaactcgcacgcgagaagcaccgcccccgctcccgccccgccccgctcctctccggccgcgcagccgctgccgcccacccgcacccgccgtcatgctccgggccgcgctgcccgcgctcctgctgccgttgctgggcctcgccgctgctgccgtcgcgggtaagcccttacgtagtccctcgccgggaccgtgcgcgaccgccttcgcccccttcccaacgcacgctcttcgtccccgcgcacccgagggcggcccgcagacgcaacacccggccggccatcccgcccttccctgcacgcccgtcccccgtgggtcctggctccgggtcacctctcacccgcctgccctcggggaggggaggtggccgagaataagggagggctctgtcttcctcggagtccacatcctcaccgcagaccccactccgcggggagggaacccccaaattaggccagttggccggagaactgagggacttggagtcgcacgacgggcgccgtttcagggcaatttcgggctgaaatgagaagcggggacgttggtggcgatttcccctgctggtgcgcggccggagtggggttgctgggatgggggtgggggccggaggaagtaggccctcttttgcaagcagcgctgtttgtctagttggttggtgttcaagttgtttaaacaggaaaacagttcagccaaataacccctggatggaagaggaacgggaataggcaaagcttggatttcactgaaatcaaggagttttaaagttctagtctgctgttgtgcaagtgacatctgaaaaatcacacacgtgatcattcatttacaaaacgactcgtgaggaaaatgcacaattctattgaccgtggtctttatttttaaaaaatttccatacaagcatgtcaaaaatatgtggatggggagactctggagaacacagacttccaaaaacaccactgactgaataattccaggaattaaagagcaaaataaacaagaactaaatgagtacttgtgtgggcttaaataaagtgcaagagatttaaataaaatgcaagagattccccccccccaccccttgccccagatttcactgcgtttttataataactgcctgctcgaagtctactgacaggaatatttcagtggacctcagtgttggaggcagcagcagctcagaacttggatacaaacccaaggttcctttcttgaaaacttctgtggacctgcatttatgactggttgtgacatctgctgcctatcaaaggggcagaaacaagatgtgcccatgttcacattgttcagactgggaacattaattttgtctaagacaaagctgggctgtctctgaaccctccttctgcacaccctcattttgcgagccagtaacatctcaactctcatgtaaaccaccctctgcgaggctgtgcatttgtactttaggctagtcgaattttcttgtcagatttttctttcttgtcagacttttaaagaaaatcagtttctagattttggtatgtctcttcttcagtgaagctgttttgaccagcaatagagggcaaatttccctttggaaatttttgtgcatttcctttgataagtccagtgtggatcaataggcttttcaagagctttagaaaagtgcatgatgaataaattaatgttaattaatcagctcctcccagtcaggaagctttaaggattaatttggaaatgagtgtgagctttgacctagctagttaaccaacttatctgcacttcagtaaaacagagataatacttactcatggggctattgggagcattaagtgggaactccacgtctagtccctattacaggcgtggttcatcttggtttccttccctttattctcttcatacaaaatgaagggtaattgttgcaaccagaaaacgtatgaataccaccttatgtatattggatgtttatggttactgaacacattcatatgtatgctaatgttatagggctgaaaaactaagtgtgtttttcataatactttacaaatctcccatccaagcaagatcaggggtcatatttggcttagaactaagtcaagaaagagtttgttgctgaataccaagatcttaatagaaaagctcttatgatgttgcataataaatatgggtattgcatataaatgtgatgttgaaacggaaatcattgttattgtctgtcattctggaggttattagtgaagtgatcttaaccttgttcttagctattattttgaaaatcacagtggacaaaacattctttaaattcctgagtgaaaatccatggcatcgctttaaaaagtttttccgtaagggtgcttaagcacataatagatgatcagtatgtatttatttggtcagtggtttcctatgcctggcatgagctgagtgaacaagcatgttccgagtaagtcctcattctgtgatcatcatgtggatcagaatctagggattttgaattgccatgtcctacaagccttagatgaggtgcctctgccttcttccctgatgtcatctcttaccacccttcctgccattcactctgtgtatccacagagtaaacagtatctgggtactccaacacctctggtccttctgtgcacaccaccttcccccagtccttggcatggctgcccccacctccaccattcagatctctgttcatatgtcaccttctccgaagcctccctgaacacccacattctctgtcacatgtccatgttttatcttttccaagcctttctgaatgcattgtgtttattcatctgtcttgcttgttgcttctcttaccaggggaaagaagctccataacagagattttaccatcttgtggatttttgttttgttttgatttgtgtctctgcactttattctaagttataataaaaggatgaaaaaatttaggtgagttatttaaaaagtaggtaggataagtggattgcaatttttttcaaattatattacctctgctatgcactcatttcataatgaattcaaagccttaaatcatcttaatctattgctgccaccttttctttctttctttttttttttttccgagatggagtctcactctgtcacccaggctggagtgcggtggcgcaatctcggctcacagcaacctctaccttctgggttcaagcaattctcctgcctcagcctcccgagtagctgggattacaggcatgcgccaccatgcctggctaacttttgtatttttagtagagatggggtttcaccatgttggccaggctggtctcaaactcctggcctcgtgatctgcccacctcggcctcccaaagtgctgggattacaggtgtgagccaccgtgcccggcctgatgccaccttttctgactcttgtgtatggcacagttatgtttcaggggaaattatctcagattaaatattagagtattttaaaataaattttgagctgggcgtggtggctcacgcctgtaatcccagcactttgggaggccgaggcgggtggatcacgaggtcaggagatcgagaccatcctggctaacacggtgaaaccctgtctctactaaaaatacaaaaaattagccgggcgtggtggcaggcacctgtggtcccagctactcaggaggctgaggcaggagaatggcgtgaacccaggagacagagcttgcagtgagctgagatcgcgccactgcactccagcctgggcgacagagcgagactcagtctcaaaataaataaatgaataaataaataaataaataaataaataaattaattaattttgaaatgttaatgtttatttttcttccaatgaaaaagtgaaatttcaattaggtattatttcaggacttcttttactgttgagaatcttttaacaatttgcttatgtgttgaaaactagttttgcagcaaggttactttccataatttttattgtgcatatcaagtatttcattccttctgtgggttatttagtgtttttttttttattattttattttattttattttattttattttattgttttgagacagggcctcactctgtcacccaggctagagtgcagtggtgcaatcatggctcactacagccttgacctgctgggctcaagtgatcctcccacctcagccccctgagtagctgggactacaggtgcatgccaccatgccctgctaattttttttttttttttttgtagaggtggggtctcactttgttgcccaggctagtcccaaactcctgagctcaagtgatcctcctacctcagccttccaaagctctgggattacaggtgtgaactgccacgcctggcctattttagattttgagagcaaatcttgagaccaaatctttaaaaaatagaccagtgatgcccaaagagaaaggtacagattaacaatggcacaccccttctatgaatatacattataacagcccttctgaagagggaagtgatgggacaatatggagatggatgtcttgaaagctagattgggaagagtgatatggagaggggatgaggtgtcagagtgcctcaatttatttgggattcagagagatcttgaagctcagagtacagccacagaatctctggattagtcagggacttggaacagccactccctcctccatacattataaatgtaataaatagcccctcttcttcatctgtctgtgccacagttgcccttgcggaagttatgtgactacaactcatatcttaatgggagaggagcaaaggtcttattattgacaatgaaaatgaaaaagaagtctttcccttttcctttgttaacatttagaacaagtatgtcccaagaatcacttcctcatgctgtgcctgttttttttttctttttttaaaaaaaagagagacagagaacttataaaggaacagagagttctattagatttgactggtaatgcaaagtattcctgtggatatgacattatcttcttttagcacatgataattttaggcagaggatctatatagaaacttttggagttatgctaagtacagcttttaaaatattcatgaggctcctgaatttctgtacgtattaaagaattataaaattataaaatccctaagagtcatttaagtgcattattaataacaacctatcattcaggactgctgtgaagaaataagagtaataggccctttgcagagagcttagtgggcaggtttttaattatattaggattatggcaaagatacttagcgttgaagacgacttagtttagtcttttgttcatgaggtgcatgaacgtcaacaatctcctcacaggagttatccagcctctatttgagcactgttcttctcaaagtaaaaatatatcttttgagtagtgcttaaaacttgtttctggggcacacactctgatcggggatctatagcatgagaaaaggggttccatgttgaattaactggagttactgctaatgctgaaatgagcctaaggagaaggcagttgaaagttttggggaagtgagtcaacagtctgttttctatttttctatacataccctgttaaaaaaaaatttttttttggtcttgtcacatgcagatgctcctcgacttagaatggggctatatcccaataaacttgtgaagtcaaaaaatcgtaagttgtccattattaagttcgggaccatttctgtattgctaacttagttattcagcaatggtctttgagtagctggttatcattatcatcatcatagtcattcccatactgtgcaacacagtagcattgtgtttgctcctccctctgtttgcctgaaatatccatgtgacttctttccctatcttttcagacacacacacacacacacacacacacacacacacacacacacacagagttggttctctgtttctctggagaaccctgactaatacaataaccaataagatagtatggaaataagacagtgtgacaatcaaggctggattgtatgaagcattgcatatccacctttgccctcctttgaatcactcactctgggggaagctacctgccatgtcataaggaccctcaagcacccctgtgtagaagtccacgtggtgaggaactgtggtgtcctgcccacagccagcaccagctcaccacccatatgagtgaggcttcttgaagctgacctttcagctccagttcagtgtttagatggctgcagccctagccagcatcttcactgtaacttcatggagaccccaagccagaatcacccagacaagcaactgcttcagaattcctgacccagagaaactgtatataataagtgtttcttgttttaagctgctaattttttacatagcaatagaccacaatacaccatctaacatattattttatgtatttgtcttccttttgcataactgcgtcaactagaacataaatttccaggagggcaggaatttcttttcctttttgttcactgtggaatctcaagcaccttgcacaattttttaacagcaccccctcagataggtactgttattcccattatacagatgaggaaactgggacacagtaaggttaaataacatgtccacgattgtgtagtcagagccagacttttctccacagtctaatgggcttaaccagagtcctgtattgacccacttctatgcatcccttccagtgttatgttcaagacagaaggatagttattattgagagtagttagatctcagacatggtctctgctgtcgtggcgttaacaatcatttgggaaaacaggatgcatatcagatagccgctatataaagaaggatctaatgagtgctatgtaaacatgtataggccatcagagggtaacacatttccttttccaaagaagaatgactacccatagttcttatgatttgtgccatattatttttaaaaccctagacacaactaattggaataaggataatttccttagtcaaaagctggctactctaggctaggcatcagcccaatatggggcctagtatatgaacgctaagcctatttgactgttgagatagtatcctcagttttcttattggccatgtatctgtacaataaatctatcatctggtagcctgagcatgtctttagtccttgttgccagaaaaggtctctcatgcataatgcagtttaaaagtcctacagatgttcaaaggagtcacaggttagaggaagataaggctgagacacttcatggagaattgacacatgagctgccactgaaataagaatataccttaatcagtgttttagagatcatatcttcagatcgttttaatttataaagtataagaactacatgttttcacagcatgccaggagcaaaagatgaaaaaaagaactataagtgcttgacttgagtgttgattatatttttgtggacttattagacattttattttatataaatggtgtttcaaaaattaattcatttttatatattgtcgagttatttgttataagtagaaaacactatctgtagttttgactagaaataatacaaaagtgcatttctgaacctgtggttcaataattgccaatgatttataaaagtgtttcaggtgtcattcagtgtggtaatgcagttccatttcacttttttaaaattccataaggtaaaagaaaaagagcaataaaaggaaccagaagaaagtaaaaagggaaaaaagaagagaaagaataatcaaaatttgcaatttagagcctctgcccttttgactttcagtttgagttttattctggaaatgacttctgtgtcctctgtcatgtctctggcagcagagaggggaaggagggaagaaacctgcctttttatcctagttcactcattcaatgaatatttatggagaccttactaggtacctgtaactgtgctttttatcatatgccctctgcctggattttttttttctttttccttttatccgtggcaacttcctatttatccagtgagctgtagcttaaatattgcctactttttgaaatgctcacttattctacaaagttatactctccttctcctacatctcatagcattttacctatcctaccttcggaatatctgtttgtctgtccgactcttccatcagactgaccacttcaagtgtagaagacttttactctttttctcatcaccccttaacactaagcattatacctaacacataggagttgctcagcaaatgtctgatatggatgatgggtagatgggtgaccaactgttgacaacttgttgaaaagtataaaagatggaattttgcatctggctatcatggagtaggagactaacactcccattaagaacaactagaaaatctagattaaattccataagaatcgtatttgctggcatcagagagctgctaaggcagtttgctgaggtgagcccaacagtctgtgtattacttttctccttaggagagctggtaaaatcttaaatagctaaaagcagagcagagtttttggcaatctcatagtgttgaggaaagaaaaaacatgagtcttagctcaccaaagaggaggtggctctagttaatatccaggctttcaactggcaatccctacggatcaaaagtctacaaagtctagaacacctcttcacatagactcagtccctgctgatcagaatcctttctaaaggaagataacatcatttagagttactacaatttttcatacacagtgtttggcatttgatagaaaactaccagacagaacaaaaaaaaaaaacaggcctaagggggtgtgagggtgcagggtaggagagtagagaacagactaataactgacctagaaattagagttttcagaaatggacttcaaaataactacgataagttaaagaatataaatactaagattgagaattttaaaatataatcaaattgaaattttaaaaatataatcaagttgaaattctataactgaaaaatcactgacattataaactcaatagatggggtttaacagcatgaaaaactgaagtaggctgggcacagtgactcacgcctgtaatcccagcactttgggaggccaaggtaggaggattgcttgagaccaagagttcaagaccagcctgggcaatatagtgacacccctgtctctacaaaaaaaaaaatcaaacaaattagccatcctgtctactcaggaggctgaggtgggaggatcacctgagcccaggagttcaaggctgcaatgagctatgaccatgccattgtaccccagcctgggtgacagagagagatcttgtcttaaaaaaaaaaaaattggagttacccttaccccagttaagttacaacttaagagtgcattaataaaccaggaagaatgagattatattatggagagtattaacagtattgaaaagagcaaaaggatatacaggacatggtaaaaagtctaacatagatgtaattttggtcccagaaagggaagagagagagaatggagcaaaagtagcatctgaaatgataatggccaagaacttttcaaaagtgaccaatgaaaataagccacagattcagaaagtgcattgaattcttagcaggatttaaaaacactaaaacaacagcatgccactgtaaaactgctaaaaaccgaagacaaagaaaatcttgaaagcagcaagaagggggaaaagcacattactgtccaaaaagcaacagtgagactgacagttaacttctcagtagaaatgatggaagcccaagcaacaaaactatagtgattcattgcttaattaacaacagggatacattttgagaaatgcaatttttcattaggcaattttgtcattgtgcaaacatcgtagagtgtacttacacaaacctagatggtatgcctactacacacctaggccacgtgctggagcctattgctctgcagtaacttgcggaacctgtatggcatgttgctatactgaatactgtaggcaattgtagcacaacataagtatttgtgtttctaaacatagcaaaagcacagtaaaaataatggtattataacctcatgggaaccctgccatatatagtctatcattgaccaagatgtcattatatggtgcatgactatgtacatcttaaatgaaagataagctgccattctacaattctgtatttgcaaagatagccatcaaacatgaagacagtataaagaccttttttctttttctttttttttttttgagacggagtctcactctgttgtcaggctggagtgcagtggtgcaatcttggctcactgcaacctccgcctcctgggttcaagtgattctcctgcctcaggctccccaatagctgggattacaggcacccgccaccacacccagctaattttttgtattttttagtagagacaggttttcaccatgttggccaggctggtctcaaattcctgacctcaggtgatccacccacctcggtgctgggattacaggcgtgagccactgtgcctggtggagacatttatagagtatcaagaactgagggagtttccagtagacctgcacttaaagaaacaccaaagagatttctttaggcaaatggaaaattatcccagtcagtaatacagaaatgcgagaaggaatgaagagcaacagagagggtaaatatgtatttaaatctaaaagaacaccggccaggcaaaacagtaataaaacaataagataaaggaaaggaagccaatgaaattaaaatattgtatgtttctaggactgcctaggaagtggtaaaagtactgatttatttgaggtattaataaatcaaggtcacatattataattttcagggtagctactacagaaaaaaaattaaagaatatataattaacaagctaaaagaaaaaggatcaaatataaaatgtttaatacaaaaaagacaataaatgagaaaaaaggaacataaaataagtaggacaaatgaaaaacaaatattaagatggtagatataaactccaattcatcgtaactacattaagtattactggaaatacttcaattaaaagacaaaggtgatcaagctggatttaaaaaaaaactacttgctgcttgcaagaattataactacttagaaaaagtgtttggcagtatctgtgaaagctgggcacatgcatactcgatggccccagcaatctacttctacaaaggtactcacaagaagtatgcatagaatgtttaaagcagcattacaatggccacaaactagaaactacccaaatattcatcaacagaagaatgaataagttcttagataagtcttaaataaaatgatttttaaagtaacatttatcaaaagaagccagacacaaaagagtacacaatgtatgattccataactataaagttcaaaaacaggcaaaactaatcgatggtgttagaagttaggttagagatgacctttggaagggatggtgagcaaaaggggttaatgttctgttcctcattcctctgataattatatgggtatgtttactttataaaaaattcaccaaaaaataaaagaaaaaagaaaaaaattcaccaacctgtatatttatgacttgtgcattgcatgtatgttttatttgaataaaaagtttagtttttaaaaagtgtatcaggaaaggtattagaattggcagcaaggtcaattcagttatattttagtatgtaagaaacttggccgggcgcagtggctcatgcctgtaatcccagcactttgggaggccaaggtggcccaatcaccagaggtcaggagttcaagaccagcctaaccaacatggtgaaaccctgtctctactaaaaatacaaaaattagctgggtgtggtggcgggagcctgtaatcccagctattggggaggctgaggcaggagaatcacttgaacccaggaggcagaggttgcattgagctgagatcgcgccactgccctccagcctgggcgacagagtgagactctgtctcaaaaaaaaaaaaaaaaaaagaaaaagacaacatgcttcaagaaggaaaggttgaacccacagggaaagggagtgtttgaagatgctgaagagatgataaattaagctacttaaggacaaataagaggattcattcattcagctgaatattttaagtccctattataatccaagagctatctgaggtgttaggaatccaaaaattaataaggcttagtccctatttttagagtggtcactgtccaggagcaaaagatgccaatgatgatgtctgtaaaaactacatatttttaatactttccttgtacgagacactgtgctgtgcaggtttgcagtatcttacctgatatttacaacaatccaatgaggtagatgttatctcccttttcctgatgaaacacaggaaataatataagttttccaaggtctcaccaccaagataaaaacataagtctgtctgacgttaaagccgtgttcctgctggtactttacactgccattggatggtgtgtggttacatgcaacagtcacaatcaattctagtggggatgtcttggagaaggtggtatagatggcctgagcctcaaaggatgagcagacagagaataatgtttgatggcctggaagtggatttgaaggaagaagagtcttttctctgaataaaggccagattttcaaagaaggtggcagaaatgtataatgaacctgagacgactgagtgctgtttctcagggaaaggatctacctgctccatatacagactttcaaccaagtgtcttgttttcagaccttcctcctacaaagctgtctggcgtctccaattcctgactctgttctggacttctttgctttgtaaatgttggtatcatgtaattttatagccagattgttttaatgtttcataatgaacattttcccaaagcaaactgcttgttgcaaaacaaccgtatctttaagccagcattgtgggagagttctagattcctgccagagttaaacccaaggtagtcatatgtgtaattcaagtgctcataaaacagctggaaggaaaaaaaatttggatataaaataaattataggtggggcacagtggctctcacttgtaatcccagcacttcggaagcccaaggtgggagaattgctgagcccaggagttccagagcagcctgggcaacatagcaagaccttgtctctacaaaaaaattaaaaaattagccgggtgtagtgttgtgtacctgtaatcccagctactcaggaggcggaggcacaagaatcacttgagcctgggaggcggaggttgcagtgagccaaggtcatgccactgcactctagcctgggtgacagagtaagactctgtctcaaaaaaaaaaaaaaaaaaaagtctgtacggatacagggagcacactgcttccttttaaaggcaagaccgctatgttgaacattccacttctgctaaaccctaattatccagaagttataagactgcgaagcaatctagcttggtggccatgtgctctgtgctatataagaagctgaggtcagtactggggacaaccaatgctctctgccatattctgagcttagtaaacttgagagtgttgaattaatttagattaatgtagctgattggtaaattctgatctttgtggtagggcttgcttctacttgtttatattaaaaaggtaatgcagaacttaggcctggcgtggtggctcacacctgttattccagcagtttaagagaccaaggcgggtggatcgcttgtgcttaggagtttgagaccagcctgggcaacgtggcgaaaccttgtctctacaaaaaatacaaaagttagccgggcatggtggcacatacctgcagtcccagctacacggaacgctgaggtgggaggatcccttgagcccaggaggcagaggctgcagtgagctgagatcgcgccactgcactccagccaggacgacagtacaagatcttgtctcaaaaaaaaaaaaaaaaaaaaaaaaaaagcagaacttaaaacataaaattgctacatatttatatagatgaaatcactaagatttttacctatatacaaacctaacagaatatggacgcttactgctttgtacttctttattcactggaattaagtaaaattctctttccattttggccagtgaaatcaactcttgcagggttacttttgcttttaatactaaccaattaaattctagagcctaggcttaaaaatgcaaatctttataataaaaagagactaagatagaggtgaaggagatgtccctttagagcagtagaggaagagttatggttctatttcattgaactacctcaaataatctactttgatgaggtgttaaattaagaacattcataagaacatcaatcttgttttcctgtgcaatatgaaaacttgtgtgaatatttataggctagcaacttgtttttcttttctctgttcttggaggtataatttgaacaacagggccttgtaaaagtcttaagacttttgaaattgttaaatactgcaaagataaccttcaaagagaagtgtgtaatcctgattggctgtcttctagatagtttgttgccttggtgataacactaatttacatgtctgaattcctaaacaacctaaaaacttgtatctttgctctgtaaagtcagaagagaatcattggcacatttctcttttcccgtatgaataatggagagaaacaaattcctgttacactggttgatttcttattttaataaagatttaacaaaggaaattgcctttgtgtgtgtgtgaaaatttagtatctaatatccttatctttgggaattcatgtaattctttttctcaagttatttaacagtgttcttcttttttctacatagactgtccttcatctacttggattcagttccaagacagttgttacatttttctccaagaagccatcaaagtagaaagcatagaggatgtcagaaatcagtgtactgaccatggtaaaaggcttattcttttctgtttgttttttcacaaattagtgaacagtgttcattaatttctttaaaaattgattttgtcacatctgacttttaactgggtcctgagatttaatgtacaacttttatttgacaacacacaagctacaggcagtggaattgggtatatatttaatcctggagttggaagacagttgttggacaatattgtccaaagcagggtctatttaacatgcatatcctgtgccaggcactgtgcttaagcacctcatatactttacctggctttatcataacaaacctatcaagtagtgtattattatcattttattgagagatgaaattaacttcagttatttgttcaagcttacacaggaaataattgacaaagccaggattttaacccagatctgttggattttaggcttagagttcttaatcacttagttattctgcctggaaagtgctgaagtcagattacataggaccatgaagtaagactgtagtaaagaagagtacattaaggaaaaattataaaaaggctatttgctagaccatacactcagtgttctctaggaaatagatttataacatcacattgtaaaatatttgaagtgtctttttttttttttttgagacagggtcttgctgtgtcccccaggcttgagtgcagtggcatgatcatagctcactacagccttgaactcctgggttcaagtgatcctcccatgtcagccccctgaatagacaggactacaggcgtgcgctagctattttatctgtttgtttgtagagacagggtttcactatgttgcccaggctggtctgaaactcttggcctcaagtgatcctctcaccttggccttccaaggtgctgggattacaggtgtgagccactgcatccagccaaaaaaatgtctttttttttttttttttttttttttgtgagatggagtctcgctctgtcgcctaggctggagtgcagtggcgtgatctcggctcactgcaagctgcgcctcccgggttcacgccattctcctgcctcagcctcccgagtagctgggattaccagcgcccaccaataggcccggctaattttttgtgtttttagtagagacggggtttcactgttggccaggatggtctcgtctcctgaccttgtgatcaggatcaagtgattctcctgcctcagcctcccgagtagctgagattacaggggtgcatcaccacacctggctaattttttgtatttttagtagaaacaaggtttcaccacgttaaccaggctggtgccgaactcctgaggtcaggcaatccacctgcctcagcctcccaaagtgctgggattacaggtgtgagccaccatgcccagcctaaaggtgctttttattactgttatattcttcaacaataacatttggaagaatgtatcaaattgtactagggataccttaatcaaaatcatatacatttattatagaaggaataaatgtaaacaccaaagttattaatcttattaaagcgccacatagtacttaatacatatatgttgtttccctttgaaaaagtcttacatgaacattggctctgtgaaaacaagacgaaatttccaacatgccaatatggttttatttcaaggcagttgaaataaaataatagcaattttgctggtcttagaatatttttaaaacaagttttcccctgcaactattttctttcctttcttcctttttcttcttcctcttttctttctttctctctcttttcatttgttctttctttctttcccttttttttttttaatggagtctcagtctgttgcccaggctggagtgcagtggtgtgatctcagctcattgcaacctctacctcctgggttcaagcaattctcctgcctcagcctcctgagtagctgggattacaggtgggtgccaccagacccaactaattttattttattttatttttttttggtagggacaggatttcgtcatgttggccaggctggtctcaaactcctgacctcaggtgatccacccgcctcagcctcccaaagtgctgggattacaggcatgagccaccacgcccagcccctacaactattttccatcccttaaaaaaaattatgtaaagtatactgagacataaacataatttcaatgacataaatcatattagaggctgagagccaagacatgtccctgtaaccttattagcttgtatgaactgtaagatcaaagtttattgtccagctctaatatatatagaaagttaagcaactatttattatttttatgtacctagtgattttcactgattctggaatatgctgactttttttctctttaaaataatttcaggagcggacatgataagcatacataatgaagaagaaaatgcttttatactggatactttgaaaaagcaatggaaaggcccagatgatatcctactaggcatgttttatgacacagatggtaagtgatatttacctcgtgggacgtgactttgttgttctttttttaatgtagcaaaactgatggcttttcaattctctggttcaagttgatgattcctgtggcctccaggtgttaaattttcagtgtgcacctatgatcataccacagcttgtatctgcatagctcaatgagttacacagatgaaagccacagttgattcttgtctggattaaaatgtggctctaaggctcagtatttttgtaatatagcatgatcataaaggtaagtattagtgtctgcaactaataggctaaggatagactaaattatcacggttctattatataaacatatttatcattactgtagacaaagtacatatgtacaaatataaaagtacatatgtaaacattttcaaatgaaatgaaattactaaggttactattaatatttaactatttttcttgacttttttcccatgagaatattatacatgtatatatttttgcgaaaattggatcatcttcagaaacctcttaaatcgatagcttttgatgatttggctgtacacaggatatattcctccttctaattttaatcctacaatcagaagtttagaccagacataccttagtaactttccacctttaatcctacaccctttttggcttgaactccttttaaaactgtcataatgttgaaaatattccttaattgacagatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctagtaagtatgaccgaaggtgtttttccattctagaaggggcagagaaagtaggactggttttaaaataattttgagataagtgtgcctcaataaatagaataaggctaggcacagtggctcacgcctgcaatcccagcattatggtaaactgaggcactggattgcttgagcgcaggagttgaagaccagcttgagcaacatggcgaaaactcatctctacaaaaaatacaaaaatcagctgggcatggtggcgtgcacctgcggtccgaaatacatgggagcctgaggtgggaggatcaattgagcctgggaggttgaggctgcagtaagcctgggcaacagagcaacactctgtctcaaaaagaaaaaaaaaaatagagggaataagacttcagaatatattaataggatggtttgctcttaagtctcttggaactcaaattgtaattctcttacatttaggaggtaaggccagatcacatgaaataggcatacttttattctgtctgcttagtgtttccaagaggtcaaaaatttcttgttcagaggaattattataatctctttgcatgtgatttgagtgagcagaagtggtgaagagtaatcatgtataattccagaattctcgtgttactttacaaggaattagattctccataagaacatcctttgtttaggcaaacaaaaactacatcatagtttataatttatttggaattaaaatgttttcttctgacccgtcagaaggttttttttttttttttttttttttttgcgatgcagtctctctctgtcgcccaggctggagtgcggtggcgcgatcttggctcactgcaacctctgcctcctggcttcaagcgattctcctgcctcagcctcccaagtagctgggactacaggcacccgccaccatgcccggctaatttttgtattttttagtagagatggggtttcaccatattggccaggctggtctcgaactcctgaccttgtgatctgcccacctcagcctcccaaagtgttaggattacagacgtgagccactgtgcccggcccagaaggttttttaatgaacagaagttatcctggcccacaacagtattttgatgttatttgttaaaacactacacagtttgttattctaagttttagaaagtaactgttttctcattaaagaaaattaaattttcaaaatgtgggctgggtacagtggctcatgcttgtaatcccagcactttttgaggctgaggcagcctgggtcaacatagcaagaccaccatctctacaaaaaaagtttttagtttttttaatgtttcaaaatatgaatttagggtgcttaaaacatgatataaacaaccatttccccattttgcatcctcagtattttccccttctacttcaactgtaaaccaaaactccacatcctgctttatttcctttctttacatatggaagtatgtacatttctttacttgctacagcaggatccttgactactgttagttttacagtgattatcaaaatgttcgtcatagttttatggtagcagtattcaacttcaaatatttgttttttaaaaaattggttcttttcaggttctgcacctcagggtgccattacataaacaaagtttttaattaaactataacttcaaaacatcagaaatagtgagtacatccatactaaggtagttagggtcacagttgtttaattttatgacatttgttttctagactctccaaccagaatcctcttttttcctttacctcattattgtcttttcctcaagatcccgtattcttttccaccaaccaagctaatgcatgttgttagccctctacatctttctttaaaaactaagttcaaaatagacaacagagacaactaatgaggcaaagaaatatggattttaggctatgaatatattgatattaagctactaataaaagggtttatttttaatgattgtgaacatgtttaaagtcaatcctataataaagcataattaaattctaatttttattttcttttgtattttagtcccatacaaaaggaaatatttatcaggtaagtaatgatttggtctttaaatttttcccacaattaaaaatatatcaaaattctggcatattaaaatacattacagatccctttttaaaattatgtaatataaagtagaacaatttttataagaatgtagaaaggtctcttagtaataagactcaacattttaaatatattctacaatatgtagttttgtctggcttcttttactcgggaatatttttttagattcatctgtgttgtgcacattcactgaattttttccctaagtgtcataatactttataaatatatcatatattgtccattctcttgtttatggatatttggatcacttctagttttaaactaatttgaatagaactgcctatgaacattctcccacatcttcttttggacctatgttctcattttctttgagtatattctagaaagtagaaagctgggtcatagggcaagtatatatatttagatttattagaaaccaccaaataatttttcaaaatgcttgaatcatgttactactggcagtgcgtccaagttccatttgctccatgttcatcccatgatggtattgtcaatttatttttagccattcagatcaggtgtgtagtgatcttcttgtggctttaattttcatttacctgataatgatgttgagcacctttcagatgattatttatttgtatatcttttgtgaaaaggcttttacccatttgcggagtgcagtggtgtgagcttgactcaccgcagtcttgacctcctgggctcaagcgatacttccacctcagcctcccaagtagctgggactacaggtgtgggccgccatgcccagctaactttttttgtacagatggagtttcaccatgttacccaggctagtctcaaactcctgggttcaaaggatccacccgcctcagcctcccaaagtgctgagattacaggcgttgggccgccatgcccagccacccatattttttttgagttgcctatatttttggtaggaatttttaaaacaaattttgtttctttaaatttggatatgggccgggctaaataatacaaaacttagccgggcacggtggcgggcgcctgtaatcccggctacttgggaggttgaggcagggagaattgcatgaatttgggaggcggaggttgcagtgagccgagatcacgccactgccctccagcctcggcgacagagtgactccatctcacaaaaaaaaaaaaaaaaaaaaaaaaattggatgtgagtcctttgccagttttctatttgggggttactacgatagaactatgacagaggaggagaaaagaagtgttgggtttgtgtgtatacaggaattacatcctcattaatctataggaagaagacaataaataacaagcattagtgtggatatgcagcaattggaactcttgtggtaggaatgtaaaatggtgcagcctctgtgaaaaacagtgtggtggttcttcaaaaaaaattattaaaagaattaccgtatgatccagcaattctgagtatgtatcccaaataactgaaagcaaggactctttaatatccgtacacccatctgtttgatatttgtgtatctatgttcatagcatcatttgccatagccaaaaggtggaagcaacccaagtgtccactgatgaagggataaacaaaatgtaatctgcacatacaactgagtatcatttagccttaaaagagaagaaaattctgacacgtgctacaacatgcataaacctgaaggacattaggctaaacaaaataagccaggcacaggccgggcgcagtggctcacgcctgtaatcccagcactttgggaggctgagaccggtggatcacctgaggtcaggagtttgagaccagcctggccaacatggcgaaatcccgactctactttaaaaaaaaaaaaaaaaaagttgaaacccggaggcagaggctgcagtgagccgggatcactccactgcactccagcctgggcgagacagagcaagcctccatctaaaacaaaacaaaacaaaaccaccacaaaaaaaaaagaaaaataagccagtcataaaaggccaaattctgtataattcttacctatagtatgaggtacctaaagtagtcaacctcaccaagaaactctttgccaagggttaggtcaggaagaaatgggagtttaataggtacagtttccattttacaaaatgagttgttctgtgaatggatggtggtgatgtagcacaacgaagtgaatgtacttaatgctactgcacacctaagatggtatttgttacatgtatttctttaaattacatttttaaaaaatcaagcagcggaagcatgttatttagacatggagacagataaatagctgaagcaacagtagtagttaactttaaggaatggatgttcaggtgagtagtggagcaggagcacagtattttcactacgtatcttctagaactatgacttttaagctacgtaaatgtattactttggaaaaaataaagatacacataagttggaacatttagaaaaaaataggtctgtggcttcaaaaacgaatagttgaaagatctggcaacatatggtcttcagactacaagaaaagtaatctctgaattcattacatgtatgcatgtatatatgtgcatgtaaatctgtattaaatatgtgtatgtaatttagtgagtaaacactatgtgtcaggctttctcctaaagttagacattgaagacagagcaatgaacaaaacaaaagcctctgctctcaagcatacattctagtgggaagggtaaaacaaaactatgtgagatggtgacacatcctatagggaaaacaggcagataaggatgaattccagtgcaagaagggaggtgttgcactttatttttatttttagtacagtggccagggaaggcctcatttaataaggccatgattttgcataatgaagacctaaagtcaaagtgaaagcctgtcatgtagatagggattaggcttagtctgactttaaggagtaaaacagtagagaacatggagttgaagcagcaaggaaacaacccaacagttaagtgtagctgccatcttggcaaacagggagttccccttcataccaagggtaagctggaagactcccttgggaagaatactttcaacagaattaaggtgtccatagtcggatgcacctagtgtggcaagcacacaggaaaggtcaagttagtccaagctgggttcagcaaaggttactggagaggatgcaggagttaaatcttcaaggataagtagacatcaggcaggagacagaggacaggttatctgaggtgacaaatcaggaccaaggtgaagggttgaatttttgctgtcacttactaaatggacttgggcaagtcatttaaaccctgtgcctcagaattaaatgcacagttggcactctcacctgaggctacagcctaacaaaatgtttcatatagtttttgaggtggattatgtatagttcgggcctcagatgatcagtgttagaactagagcgttaaggtagcagtcagatagataaataggaaacagaagaaaaatgtgtagtcttcacaatttatagagaggaagtctataaatttccagattggttgactggacagtggctatttatttgcaggaattcaggttagaagaaatttggaagtagggctacacacacatatttatctatggatttacataaaagacttgaatggttgagatcaaccatggaaagctttgagtccacaaagatgactaggaagcagtcgtctgaaaaatagagcagtcagggaagccaagtgagtaggagataacttcaaggctggacggctaacaatctagttgccccaggatgaactaaaacatccacgtgtttaagcatgtagtcataaagagatcattggtgacctctgccacagaaatttctgaagaaatgttgagaaaatggattaaatgggcttaagaaatgaaaaagtgagcttgtttcagtgtttgctgtttcctcaagtaaggaacattgttaggctaaaaagtatttttcctaaattttgtttctttaaaaaaaacaggatttcatggtaaatagctattatatgctgtcaagcaagcaatggacctttcctttaattaatgaggtagataatacatttcctaatgtaaaactatcctaacctccagatataaactacacattgttttaatacactgcttatcagttgccttgacaattgactttgtcacacattttagtagtatgcagctagctaactaaaacccctggaggcaactttggctaaaggtcacttgcagaatgtgaaaagcagcatatatacttactgtgtctcgcccatcttggacactgtccagttcttaggatacccggtcttttaaagctattatgattgagtctgtaaatatttctcaattactatgatctttcagacttttctatattactatcatgtaattcctatttaaagtagatgctaccgattatgaagccattattgtacaatcattagtacactgaaataaagtgtgttcacactggtaaagttgaggttctacactgaaaatggactatgttaattctaataaaactaactacggggctgggcacggtggcccacacctgtaatcccagcactctgggaggccgaggggggcagatcacttgaagccaggggtttcggaccaacctgggcaacatggcaaaactgtctctactaaaaatacaaaaattagctggacatcatggcgtgcgcctgtcatctcagctacccaggctgacacatgagaatccctggcacccagaaggcggaggttgccgtagccaaaaactatggaagaaccccatttgaactcagtttacggaattcttggctttttttcattttaatgtctttaataataccaccaccaaatccaccctcacaccttaccaaatctttactaccaaattttatttcagaattaaaaagatagttcacttggggccatttcctcactcctcccaaaaaatcctgtcaatatttgaaaagtatcaatggctctccctcactttaacaaataatttggccaaatttctctcactaatcttaaattcttccaggtctcattttaacaagcaccagttgaaataatgggtattatgaatatatttaataataaaaaatctgtcaaaataattctgaaaagttttcacattctttagagtcgattactatacataatacaagtacctgcccaaaagtatgtttgttctcctaaaccaggttattttcctcaatttattaaactggattcttatagaaagattacaagggtttgaataggcaagaactgcaaagttgtgcatacttacagaaatttagggaaacacacaaagaatgaatgaatgcaaagacacacaaaaccaggtactttactgcaggtcttggtgtttttttcagttaaacaaatcttaaatccccaaagccaggtgaaccattaaacctcaataatgtaacacattatctaaatactagaattcctaaagggaacagtaggtctgaatttgtagtcttagatcagaaagtaaagcgaaggaagacaaactggagcatggtatatacttgcgtgatttccactatggggtcttcagtagagtggagaagtgcacaacttcctagaaaatttgagtactattcttcaggctaagaaatataaatcttgttagtaacaaatgcgaaatttttggttttctcatcagtgtgggaaattctcaacacagagaaatgtttacacaaaactacagttgtgctcatcaaaatatacttgcaacttgtgtaccctaatttcccaaatactcttttgtcttttccttttcagataaccacattttaatatcagcattggtgattgctagcacggtaattttgacagttttgggagcaatcatttggttcctgtacaaaaaacattctgattctcgtttcaccacagttttttcaaccgcaccccaatcaccttataatgaagactgtgttttggtagttggagaagaaaatgaatatcctgttcaatttgactaagtttttggtaatcttgcactaagacatcaacaaaatgccctggcagagataacttgggaaagattttaatataaaacttgacattggatattagagctttaatggtattccttattccagtaacatttttatgtactcatctgctgtgaaaagtctttaggttcattaaaaaaacaggttttagaaatgatcttagatctaatatagtgattttaagcatcccgtcaaaggcagaatctgtcacttgaatgaaggaaagcttaaagcccaagcagataaaaataaaagcccagcctatttgtcttgcctgctgtatcttccctatttagttgacccactttagtttatatgtttattagtaaacatgaaatggggaataagtgattttaagtacatcccatacatttaaatatctttgataattgttatttttttggcagataattcctctagaatgtgtatctttttatgatttagatgaagaaaattttacaacttttaacaccccacaccaattttagtttcattacttttacacacaccattttatcacaaatgactcaagttttaatgaatgtttataaattatttgaaacaaaatatgatcgctgtgtccaggatggcatagagaaagctggcaattaggttaacacttacatattatagtgcccctttaaggatttctctcttgccaccataccttttgtactttcccctatacaagatgtatctcattctcctcaagcatttataaatttttccttcaatgacatgaaaactgtgcaagcaaaaaccgaagaaaaacacttaagtacaactgtagtgacagtgatcaaagttttcagtgcatttattgtacattttaagaaaaaggtgaaaatcatttggggagtaaaaaaatgaaaaagctgaaacgagtaattttcctcaccatcaataaaccaaaaacaggaaagataaagaatgtataaatttcacgtaaattagtcacgtatcacttatcaatggggatacgttctaagaaatgcatagttagggaatcttgtgtgaaaatcagcttgtatttacacaaacccagatggtagagcctattttgtcccaaacctacacagcatgttactgtgctgaatactgcagacaattgtaacacaatatttgtgtatctaaatatagaaaaggtacagtaaaaatatggtctactaaggaaacactgttctatatgtggtccattactgactgaagtatactgtctagaagtctgaggctcaaagaaaagtaatccctcttctgaatccacaccccatcaattatcttactttcttctggggagatagatagatatactatctcactagcttgactaatggcaacaaagttccagcttgtgtagtctctttttattgaccacatgaatcgaaaacactcatcacaattaatggcactatcattaatgagacatgagtaactaaaaagtgatagaaaactattaacagtgcggctacatggtactgaaaatgcaggcattacaccagctgttacacaagcacaagcatgctctgtaagagctttacatttctgagattttgtatagtgattgagatgtctattttattattgatagactattactaatgtcaatattgaacactaccctggaattcctgcctggttttcctacccaaattgtaccactccttgaagaactacaggcacagtaaaaaaaatatggcgtattatgtgaactaaaagagttctaaaggagttcttaaaggagtggtagaatttgggtaggaaagtgattaagtccaacttaaaaccaacagtctcaaacgtctacaactacaatgtccaatgagccactagccacatgaggctatttaagtaaatttagtttaaaatccagttttcgaattacattagccacattgtcaagtgttcaaatcacaggtggttagtggctactgtactgggcaacatacattatagaacattttcattataggaagttttattgggcagtgctgctcttaaatcctaccttccactcaactcccatacaactttcttttgtacattttgatactttctacctaatggcagctcttccaaaatagctgctttaaactctgatttaattttcaatatttggtttcatttttcaacaggccaagaggcctctggtaatgaagtgctatatatatatatatgatggagtctcactgtgctgcccaggctacagtgcagtggctcgatcttggctctctccaatctccgccttgcaggttttcaagcaattctcctgcctcagcctccttagtagctgggaccacagacatctgtcaccacacccagctaactttttgtatttttggtagagacggggtttcgccatattgactgggctggtctcaaactcctgacctcaagtgatccacccaccttggtctcccaaagtgctgggattacatgcgtgagccaccacacttggcctacattttttctttatataccagaacatctataacaggcaccttatctactcattagtgaagagataattggattacacaggcaggcttgtttactacatccagaatgtagaaactgctttcttcaacatcttggttctagctagtaataacaatataattctttggcagatattcagaataacattttaaactacattttcttagaaaattgcattcttgtagtgagcagtgtatggtctcttttgttcagaatttaaaactgataaccaatgaaagccttttctcttattcctctaccgtcatttacatgataatctgaagctaatatgacaatatttaaatactaagtggtactagggaactacaagaatactgtaaagcttaagccattgttatcactgtcatttagcatttaataacaaaactatacagaattatgtgcataccaatgaatgttttgtaccatctagttaaattttttaaataaagttttatgggttaagcagaagacaactgtcatactgaattttattaaaagtatatatacttcaaattcaaagcatcccttaggacccacagaatatattaaaactaccacccttaaattttatatttttgctttaagacagacaatgcaaaggtaactggcaagaggtgagcaaatgttttagaacatttatattattgcttaaaatgagatttgaaattgtaataaaattcttggttatgaagtctgatgtcttctttgagcaccagtttaaaaggaaacatttcaaacagtaaaataaatcagtgtgtactttattttgagccaatgttttacataagaccaacaaactaagtgtgggcaaactgttcaatcacttttgagaaattacatggttcctatttaggtttgaaaaagtagtttgttgccataacatcagaccttaaggtgttttaaagtgtatcctaagggtactgacagaagaaaaatacagtgttatggaagtatacacaactcatgaccacacagtatagatccattcatggtgaataatgttcatctgttctccaaaggtagtgaaaaatattaagtcatcaaataaatgctcagaattaccaaggaacagttaaaaaggaccaggatccaaacaggttatttatatatttatctataaacaagtgacaacactaaaaaacacattgaaaaccagtgttttaaactctgaatgtgggatttaaaaatattccattaacttgagattgcaataatttttttaaaccaaatagcatttgactggcaagtctcatataatttacagctgtaatattcctaaaaggaatatctacatactatagcctatcaaatataatctgcattttgcaaatcaatacaaaaatctatccatcatttatacattttatatttatatactttttaaaaattagattttattgctaggtgaatttggaattcaaattaaatttggaaacttaataatgctcttctcataaccttctccccattctttttttaatttttaaacactggaagcaaatgattatcaaaatgagactgcaacttaaggcattttaaaagaaaaaataattaggtgccatatagcattttatttcaaaagtatattttgtcccacttttctcactagcaagagtaaaacacaaaccttttttcataaatatgactacagtaatcataacacaaaaaagggttgataggcattgcttagatatttaaaacaagggtaatactttcccactcacctaaaagaaaaaacctttttgattaccagtttataaacatcggatttgctatgttaaaaagtccagcagaattttaattcagcaacactggagaatgaatatatatatacaggcatacatgtgtatgtatatattcattttatatatagtcaatgtatttattaggcattcccccacaaaagtaatttatatttaattgccatttttaataaacacttatgttcacagatcatccatctgtcttatatgaagttaggcaatatcaaatgtcctgttatggtctccgtcttcttcggaatcatcctctgaagctgttgaaagaaaacaggatgatcaagaatgattttaagggggataagaatgggaagtgtaaatatgaagccagaacagcaatgacttgtaaaaacaaaaatcccaatttagtatattgtttgatcaaaatgataagttgtatgacagggttagggtggtctgcctgtgggtgatacccaaccacaattctccccgcccccacaccgttttgtaatgttatatcaatttttataaaaatgaaagtgcttaaaaatgtggttcaagagcatcttctaaaatgtttctgagatcaatctgtgttcatagtagctaatgatatataaacactaggaaaaaagtcaacttaaggcaagtgctctcacacacctgagtaaaatctggcttattaccgctgcatgtttggtgcatccgatgagaatgaaaagaagtgtacatagaataagaggttatgggtgttcttgcaatttacatcaaagctatgtctatacaatgccaagataaaagttc

(19) United States
(12) Patent Application Publication
(10) Pub. No.: US 2006/0281672 A1
(43) Pub. Date: Dec. 14, 2006

(54) Dec-205 (ly 75)/dcl-1 intergenic splice variants associated with hodgkin's disease, and uses thereof

(76) Inventors: Derek Nigel John Hart, Queensland (AU); Masato Kato, South Brisbane (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/537,839

(22) PCT Filed: Dec. 6, 2002
(86) PCT No.: PCT/AU03/01634

§ 371 Date: May 18, 2006

(30) Foreign Application Priority Data
Dec. 6, 2002 (AU) 2002953223

(51) Int. Cl. A61K 38/17; C07K 14/705; C07H 21/04; C12P 21/06

(52) U.S. Cl. 514/12; 530/350; 435/69.1; 435/320.1; 435/325; 536/23.5; 530/388.22

(57) ABSTRACT

The inventors have identified intergenically spliced DEC-205/DCL-1 mRNAs, which encode the intact DEC-205 ectodomain together with an additional carbohydrate recognition domain, a transmembrane domain and a cytoplasmic domain derived from DCL-1. These DEC-205/DCL-1 intergenic splice variants were identified on Reed-Sternberg cells and thus have application in the therapy and investigation of Hodgkin's disease.

### FIELD OF THE INVENTION

The present invention relates generally to a novel lectin receptor and to derivatives, homologues, analogues, chemical equivalents and mimetics thereof and, more particularly, to novel splice variants of DEC-205. The present invention further relates to a novel lectin and to derivatives, homologues, analogues, chemical equivalents and mimetics thereof and, more particularly, to a novel type I C-type lectin, herein referred to as “DCL-1”. The present invention also contemplates genetic sequences encoding said novel molecules and derivatives, homologues and analogues thereof. The molecules of the present invention are useful in a range of therapeutic, prophylactic and diagnostic applications.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Hodgkin's disease accounts for 15% of all lymphomas, but less than 1% of all cancers. It is diagnosed in 7 per 100,000 people annually. Hodgkin's disease can occur at any age, but is rare in children. It most commonly strikes young adults between the ages of 20-30 years and adults above the age of 50 years. Hodgkin's disease is more common in higher-socioeconomic groups and more men are affected by the illness than women.

Hodgkin's disease is characterised by the presence of Reed-Sternberg cells. These are malignant morphologically distinct cells, the presence of which is used as a diagnostic criterion of Hodgkin's disease.

In nodular lymphocyte predominant Hodgkin's disease, Hodgkin and Reed-Sternberg cells occur amongst a background of polyclonal B and T cells. The proliferation of these lymphocytes is postulated to be mediated by malignant Hodgkin and Reed-Sternberg cells. Hodgkin and Reed-Sternberg cells exhibit characteristics in common with antigen presenting cells such as activated B cells and dendritic cells. For example, Hodgkin and Reed-Sternberg cells lines, such as KM-H2, L428 and HDLM-2, express cell surface molecules required for costimulation/proliferation of B and T cells (MHC class II, CD40, CD80 and CD86), cell adhesion molecules involved in APC-T cell interactions (LFA-1, CD11c, ICAM-1-3), and produce inflammatory cytokines (TNF-α and lymphotoxin) and non-inflammatory cytokines (e.g. CSF-1, IL-5 and IL-13), all of which may contribute to the pathology of Hodgkin's disease.

In light of the unique distribution and characteristics of Reed-Sternberg cells, there is an on-going need to investigate and define the phenotypic and functional characteristics of this population of cells.

In work leading up to the present invention, the inventors have studied the cell surface molecule expression of Reed-Sternberg cells with a view to identifying molecules which may provide useful immunotherapeutic targets. In this regard, the inventors have surprisingly identified novel alternatively spliced DEC-205 mRNAs which encode the intact DEC-205 ectodomain plus a unique sequence encoding for an additional carbohydrate recognition domain (CRD), a transmembrane domain and a cytoplasmic domain derived from a newly identified type I C-type lectin termed DCL-1.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

The subject specification contains nucleotide sequence information prepared using the programme Patentin Version 3.1, presented herein after the bibliography. Each nucleotide sequence is identified in the sequence listing by the numeric indicator <201> followed by the sequence identifier (eg. <210>1, <210>2, etc). The length, type of sequence (DNA, etc) and source organism for each nucleotide sequence is indicated by information provided in the numeric indicator fields <211>, <212> and <213>, respectively. Nucleotide sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing. A summary of the sequences detailed in this specification are provided immediately prior to the examples, in Table 4.

One aspect of the present invention provides a novel nucleic acid molecule in isolated form wherein said nucleic acid molecule comprises a novel DEC-205 intergenic splice variant.

In another aspect there is provided a novel nucleic acid molecule in isolated form wherein said nucleic acid molecule comprises a DEC-205/DCL-1 intergenic splice variant.

Yet another aspect provides a nucleic acid molecule or derivative, homologue or analogue thereof comprising a nucleotide sequence encoding an amino acid sequence substantially as set forth in SEQ ID NO:2 or SEQ ID NO:21 or a derivative, homologue or mimetic thereof having at least about 45% or greater similarity to at least 30 contiguous amino acids in SEQ ID NO:2 or SEQ ID NO:21.

Still another aspect provides a novel nucleic acid molecule or a derivative, homologue or analogue thereof in isolated form comprising a nucleotide sequence substantially as set forth in SEQ ID NO:1 or SEQ ID NO:20 or a nucleotide sequence having at least about 50% similarity to all or part thereof or a nucleotide sequence capable of hybridising to the sequence set forth in SEQ ID NO:1 or SEQ ID NO:20 under low stringency conditions at 42° C.

Yet still another aspect of the present invention contemplates a nucleic acid molecule or derivative, homologue or analogue thereof comprising a nucleotide sequence substantially as set forth in SEQ ID NO:1 or SEQ ID NO:20 or a derivative thereof or capable of hybridising to SEQ ID NO:1 or SEQ ID NO:20 under low stringency conditions at 42° C. and which encodes an amino acid sequence corresponding to an amino acid sequence set forth in SEQ ID NO:2 or SEQ ID NO:21 or a sequence having at least about 45% similarity to at least 10 contiguous amino acids in SEQ ID NO:2 or SEQ ID NO:21.

Still yet another aspect of the present invention contemplates a nucleic acid molecule comprising a sequence of nucleotides substantially as set forth in SEQ ID NO:1 or SEQ ID NO:20.

A further aspect of the present invention provides a novel cDNA or a derivative, homologue or analogue thereof in isolated form comprising a nucleotide sequence substantially as set forth in SEQ ID NO:1 or SEQ ID NO:20 or a nucleotide sequence having at least about 50% similarity to all or part thereof or a nucleotide sequence capable of hybridising to the sequence set forth in SEQ ID NO:1 or SEQ ID NO:20 under low stringency conditions at 42° C.

Another further aspect of the present invention provides a nucleic acid molecule or derivative, homologue or analogue thereof comprising a nucleotide sequence encoding an amino acid sequence substantially as set forth in SEQ ID NO:5 or a derivative, homologue or mimetic thereof having at least about 45% or greater similarity to at least 30 contiguous amino acids in SEQ ID NO:5.

In another aspect there is provided a nucleic acid molecule or derivative, homologue or analogue thereof comprising a nucleotide sequence encoding an amino acid sequence substantially as set forth in SEQ ID NO:8 or a derivative, homologue or mimetic thereof having at least about 45% or greater similarity to at least 30 contiguous amino acids in SEQ ID NO:8.

In still another aspect there is provided a nucleic acid molecule or derivative, homologue or analogue thereof comprising a nucleotide sequence encoding an amino acid sequence substantially as set forth in SEQ ID NO:11 or a derivative, homologue or mimetic thereof having at least about 45% or greater similarity to at least 30 contiguous amino acids in SEQ ID NO:11.

In yet another aspect, the present invention provides a novel nucleic acid molecule or a derivative, homologue or analogue thereof in isolated form comprising a nucleotide sequence substantially as set forth in SEQ ID NO:4 or a nucleotide sequence having at least about 50% similarity to all or part thereof or a nucleotide sequence capable of hybridising to the sequence set forth in SEQ ID NO:4 under low stringency conditions at 42° C.

In still yet another aspect, the present invention provides a novel nucleic acid molecule or a derivative, homologue or analogue thereof in isolated form comprising a nucleotide sequence substantially as set forth in SEQ ID NO:7 or a nucleotide sequence having at least about 50% similarity to all or part thereof or a nucleotide sequence capable of hybridising to the sequence set forth in SEQ ID NO:7 under low stringency conditions at 42° C.

In still another aspect, the present invention provides a novel nucleic acid molecule or a derivative, homologue or analogue thereof in isolated form comprising a nucleotide sequence substantially as set forth in SEQ ID NO:10 or a nucleotide sequence having at least about 50% similarity to all or part thereof or a nucleotide sequence capable of hybridising to the sequence set forth in SEQ ID NO:10 under low stringency conditions at 42° C.

A further aspect of the present invention contemplates a nucleic acid molecule or derivative, homologue or analogue thereof comprising a nucleotide sequence substantially as set forth in SEQ ID NO:4 or a derivative thereof capable of hybridising to SEQ ID NO:4 under low stringency conditions at 42° C. and which encodes an amino acid sequence corresponding to an amino acid sequence set forth in SEQ ID NO:5 or a sequence having at least about 45% similarity to at least 30 contiguous amino acids in SEQ ID NO:5.

In another further aspect the present invention contemplates a nucleic acid molecule or derivative, homologue or analogue thereof comprising a nucleotide sequence substantially as set forth in SEQ ID NO:7 or a derivative thereof capable of hybridising to SEQ ID NO:7 under low stringency conditions at 42° C. and which encodes an amino acid sequence corresponding to an amino acid sequence set forth in SEQ ID NO:8 or a sequence having at least about 45% similarity to at least 30 contiguous amino acids in SEQ ID NO:8.

In still another further aspect the present invention contemplates a nucleic acid molecule or derivative, homologue or analogue thereof comprising a nucleotide sequence substantially as set forth in SEQ ID NO:10 or a derivative thereof capable of hybridising to SEQ ID NO:10 under low stringency conditions at 42° C. and which encodes an amino acid sequence corresponding to an amino acid sequence set forth in SEQ ID NO:11 or a sequence having at least about 45% similarity to at least 30 contiguous amino acids in SEQ ID NO:11.

Yet another further aspect of the present invention contemplates a nucleic acid molecule comprising a sequence of nucleotides substantially as set forth in SEQ ID NO:4, SEQ ID NO:7 or SEQ ID NO:10.

Still another further aspect of the present invention is directed to a isolated protein selected from the list consisting of:
(i) An isolated DEC-205 intergenic splice variant or a derivative, homologue, analogue, chemical equivalent or mimetic thereof.
(ii) An isolated DEC-205/DCL-1 intergenic splice variant or a derivative, homologue, analogue, chemical equivalent or mimetic thereof.
(iii) A protein having an amino acid sequence substantially as set forth in SEQ ID NO:2 or SEQ ID NO:21 or a derivative, homologue or mimetic thereof or a sequence having at least about 45% similarity to at least 30 contiguous amino acids in SEQ ID NO:2 or SEQ ID NO:21 or a derivative, homologue, analogue, chemical equivalent or mimetic of said protein.
(iv) A protein encoded by a nucleotide sequence substantially as set forth in SEQ ID NO:1 or SEQ ID NO:20 or a derivative, homologue or analogue of said nucleotide sequence or a derivative, homologue, analogue, chemical equivalent or mimetic of said protein.
(v) A protein encoded by a nucleotide sequence substantially as set forth in SEQ ID NO:1 or SEQ ID NO:20 or a derivative, homologue or analogue thereof or a sequence encoding an amino acid sequence having at least about 45% similarity to at least 30 contiguous amino acids in SEQ ID NO:2 or SEQ ID NO:21 or a derivative, homologue, analogue, chemical equivalent or mimetic of said protein.
(vi) A protein encoded by a nucleic acid molecule capable of hybridising to the nucleotide sequence set forth in SEQ ID NO:1 or SEQ ID NO:20 or a derivative, homologue or analogue thereof under low stringency conditions at 42° C. or a derivative, homologue, analogue, chemical equivalent or mimetic of said protein.
(vii) A protein encoded by a nucleic acid molecule capable of hybridising to the nucleotide sequence as set forth in SEQ ID NO:1 or SEQ ID NO:20 or a derivative, homologue or analogue thereof under low stringency conditions at 42° C. and which encodes an amino acid sequence substantially as set forth in SEQ ID NO:2 or SEQ ID NO:21 or a derivative, homologue or mimetic thereof or an amino acid sequence having at least about 45% similarity to at least 30 contiguous amino acids in SEQ ID NO:2 or SEQ ID NO:21.
(viii) A protein having an amino acid sequence substantially as set forth in SEQ ID NO:5, SEQ ID NO:8, or SEQ ID NO:11 or a derivative, homologue or mimetic thereof or a sequence having at least about 45% similarity to at least 30 contiguous amino acids in SEQ ID NO:5, SEQ ID NO:8, or SEQ ID NO:11 or a derivative, homologue, analogue, chemical equivalent or mimetic of said protein.
(ix) A protein encoded by a nucleotide sequence substantially as set forth in SEQ ID NOs:4, 7 or 10 or a derivative, homologue or analogue of said nucleotide sequence or a derivative, homologue, analogue, chemical equivalent or mimetic of said protein.
(x) A protein encoded by a nucleotide sequence substantially as set forth in SEQ ID NOs:4, 7 of 10 or a derivative, homologue or analogue thereof or a sequence encoding an amino acid sequence having at least about 45% similarity to at least 30 contiguous amino acids in SEQ ID NOs:5, 8 or 11 or a derivative, homologue, analogue, chemical equivalent or mimetic of said protein.
(xi) A protein encoded by a nucleic acid molecule capable of hybridising to the nucleotide sequence set forth in SEQ ID NOs:4, 7 or 10 or a derivative, homologue or analogue thereof under low stringency conditions at 42° C. or a derivative, homologue, analogue, chemical equivalent or mimetic of said protein
(xii) A protein encoded by a nucleic acid molecule capable of hybridising to the nucleotide sequence as set forth in SEQ ID NOs:4, 7 or 10 or a derivative, homologue or analogue thereof under low stringency conditions at 42° C. and which encodes an amino acid sequence substantially as set forth in SEQ ID NOs:5, 8 or 11 or a derivative, homologue or mimetic thereof or an amino acid sequence having at least about 45% similarity to at least 30 contiguous amino acids in SEQ ID NOs:5, 8 or 11.
(xiii) A protein as defined in any one of paragraphs (i) to (xii) in a homodimeric form.
(xiv) A protein as defined in any one of paragraphs (i) to (xii) in a heterodimeric form.

Another aspect of the present invention contemplates a method of modulating DEC-205 SV expression or DEC-205 SV functional activity in a mammal, said method comprising administering to said mammal an agent for a time and under conditions sufficient to up-regulate, down-regulate or otherwise modulate expression of DEC-205 SV or functioning of DEC-205 SV.

Yet another aspect of the present invention is directed to a method for modulating DCL-1 expression or DCL-1 functional activity in a mammal, said method comprising administering to said mammal an agent for a time and under conditions sufficient to up-regulate, down-regulate or otherwise modulate said expression or functioning.

Still another aspect of the present invention contemplates a method for regulating cellular activity in a subject said method comprising administering to said subject an effective amount of an agent for a time and under conditions sufficient to modulate DEC-205 SV expression of DEC-205 SV functional activity.

In yet another aspect there is contemplated a method of regulating cellular activity in a subject said method comprising administering to said subject an effective amount of an agent for a time and conditions sufficient to modulate DCL-1 expression or DCL-1 functional activity.

In yet still another aspect there is provided a method for the treatment and/or prophylaxis of a condition characterised by aberrant, unwanted or otherwise inappropriate functioning of DEC-205 SV or DCL-1 in a subject, said method comprising administering to said subject an effective amount of an agent as hereinbefore defined for a time and under conditions sufficient to modulate the expression of DEC-205 SV or DCL-1 and/or functioning of DEC-205 SV or DCL-1.

In still yet another aspect there is provided a method for the treatment of Hodgkin's lymphoma in a mammal, said method comprising administering to said mammal an effective amount of a cytolytic and/or cytotoxic agent which agent interacts or otherwise associates with DEC-205 SV, for a time and under conditions sufficient for said agent to lyse, apoptose or otherwise kill Hodgkin and Reed-Sternberg cells.

Single and three letter abbreviations used throughout the specification are defined in Table 11.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | The | T |
| Tryptophan | Trp | W |
| Tyrosine | Tyr | Y |
| Valine | Val | V |
| Any residue | Xaa | X |

### BRIEF DESCRIPTION OF THE DRAWINGS

FIG. 1. Identification of the cDNA clone encoding DEC-205/DCL-1 fusion. (A) A schematic presentation of DEC-205 mRNA (top, partial structure) and two representative clones (pB30-3 and pB30-1) isolated from the DEC-205 3′-RACE product. The boxes in the DEC-205 mRNA indicate domain structures, including CRDs, a TM and CP. Wide black bars indicate the DNA sequence for DEC-205¹⁷ and wide shaded bars indicate the DNA sequence for the novel C-type lectin DCL-1 (KIAA0022).²² The broken line indicates the position of the junction between DEC-205 and DCL-1. (B) The DNA and corresponding amino acids sequence adjacent to the junction for DEC-205/DCL-1 fusion protein. Sequence of the pB30-3 and pB30-1 were aligned with DEC-205 (top) and DCL-1 (bottom) sequences. An arrow indicates the DEC-205/DCL-1 junction, apparent after gene analysis was performed to assign the exon-intron junction of DEC-205 and DCL-1 gene. SP, signal peptide; CRD, carbohydrate recognition domain; TM, transmembrane domain; CP, cytoplasmic domain.

FIG. 2. The DEC-205/DCL-1 fusion mRNA encodes the entire DEC-205 ectodomain. The L428 cDNA was subjected to RT-PCR using either DEC-205 specific reverse primer (085) or DCL-1 specific reverse primer (086) in combination with various DEC-205 specific forward primers (078, 088, 090, 092 and 094), and fractionated with 0.8% (w/v) agarose gel electrophoresis. The positions of these gene specific primers are indicated as arrows in the schematic diagram (bottom). The doublets obtained with several sets of primer combinations correspond to alternatively spliced DEC-205 mRNA (see text). SP, signal peptide; CR, cysteine-rich domain; FN, fibronectin type II domain; CRD, carbohydrate recognition domain; TM, transmembrane domain; CP, cytoplasmic domain.

FIG. 3. The DEC-205/DCL-1 fusion mRNA is predominantly expressed by HRS cell lines. Total RNA from hematopoietic cell lines were subjected to Northern blot analysis, probed sequentially with the DCL-1 (top panel) and DEC-205 (middle panel). The bottom panel shows methylene blue staining of 28S ribosomal RNA.

FIG. 4. The DEC-205 and DCL-1 gene are juxtaposed in chromosome band 2q24. A schematic drawing of DEC-205 (partial) and DCL-1 mRNA (top), DEC-205 (partial) and DCL-1 genes on chromosome 2q24 (middle) and DEC-205/DCL-1 fusion mRNA (bottom). In the top and bottom drawings, boxes indicate domain structures (please see keys in FIG. 2). In the middle panel, boxes indicate exons.

FIG. 5. DEC-205/DCL-1 fusion mRNA is translated to the fusion protein. (A) The cell lysates from HRS cell lines (L428, HDLM-2 and KM-H2), HEL and Jurkat cells were immunoprecipitated with anti DEC-205 CP, anti DCL-1 CP peptide antisera or non immune rabbit IgG, and the immune complexes were subjected to Western blot analysis using DEC-205 mAbs (M335 plus MMRI-7). The signals were detected by ECL on X-ray films. (B) The cell lysates as above were applied to a ELISA plate coated with DEC-205 mAbs, and bound DEC-205 or DEC-205/DCL-1 fusion protein was detected with anti DEC-205 CP (for DEC-205) or anti DCL-1 CP (for DCL-1). The signals were detected with OPD at 492 nm.

FIG. 6 is a schematic representation of the CED-205/DCL-1 fusion protein.

FIG. 7 is a schematic and annotated representation of the DCL-1 protein molecule.

FIG. 8 is an image of Northern blot analysis of hematopoietic cell lines for DCL-1 mRNA expression.

FIG. 9 is a schematic representation of the DCL-1 gene structure.

FIG. 10 is a schematic representation of the construction of expression vectors for FLAG-DCL-1 and FLAG-DCL-1-Ig fusion protein.

FIG. 11 is an image depicting DCL-1 protein expression in FLAG-tagged DCL-1 transfectants.

FIG. 12 is an image depicting expression of DCL-1 mRNA and protein in purified leukocytes.

FIG. 13 is a representation of the strategy for producing monoclonal antibodies against human DCL-1.

FIG. 14 is a graphical representation of the flow cytometric analysis of DCL-1 expression on peripheral blood mononuclear cells using monoclonal antibodies against DCL-1.

FIG. 15 is a representation of the genomic DCL-1 sequence (SEQ ID NO: 32). Exons are underlined, CDS capitalised and initiation and step codons shown in bold. Table 4 details the human DCL-1 exon-intron structure.

FIG. 16 is an annotated representation of the DCL-1 sequence.

### DETAILED DESCRIPTION OF THE INVENTION

The present invention is predicated, in part, on the identification of novel DEC-205 splice variants. More particularly, the inventors have identified RNA splice variants of DEC-205 which encode an intact DEC-205 ectodomain in addition to a novel carbohydrate recognition domain, transmembrane domain and cytoplasmic domain. Still further, the inventors have determined that the generation of these novel splice variants is likely the result of an intergenic splicing event which leads to the formation of a fusion mRNA comprising both partial DEC-205 mRNA and a novel carbohydrate recognition domain, transmembrane domain and cytoplasmic domain encoding mRNA sequence. In investigating these unique cistronic mRNAs, the inventors have yet further determined that the novel carbohydrate recognition domain, transmembrane and cytoplasmic domains, which are spliced together with a partial DEC-205 mRNA transcript in order to form the subject novel DEC-205 splice variants, corresponds to a novel type I C-type lectin, herein termed “DCL-1”. The identification of these novel molecules now permits the identification and rational design of a range of products for use in prophylaxis, therapy, diagnosis and antibody generation including, for example, in the context of diagnosing and/or treating disease conditions characterised by the presence of Reed-Sternberg cells.

Accordingly, one aspect of the present invention provides a novel nucleic acid molecule in isolated form wherein said nucleic acid molecule comprises a novel DEC-205 intergenic splice variant.

Reference to “DEC-205 intergenic splice variant” should be understood as a reference to an RNA product of a splicing event which results in the introduction of non-DEC-205 nucleic acid material to DEC-205 nucleic acid material. This may occur at the level of either the primary RNA transcript or the mRNA. Preferably, the DEC-205 intergenic splice variant is an mRNA DEC-205 intergenic splice variant. In this regard, it should be understood that the subject splice variant may be a splice variant of any form of DEC-205 such as any allelic form of DEC-205. Still further it should be understood that the DEC-205 encoding portion of the splice variants of the present invention may not necessarily correspond to the entire DEC-205 encoding mRNA. For example, the variants exemplified herein encode a molecule comprising the DEC-205 ectodomain (being the signal peptide, cysteine rich domain, fibronectin type II domain and carbohydrate recognition domains 1-10) followed by the DCL-1 carbohydrate recognition domain, transmembrane domain and cytoplasmic domain. In a most preferred embodiment, the subject non-DEC-205 nucleic acid material corresponds to all or part of the DCL-1 gene or its transcribed RNA product. The fusion/splicing together of all or part of DEC-205 nucleic acid material with DCL-1 nucleic acid material to form a novel DEC-205 intergenic splice variant is herein referred to as a “DEC-205/DCL-1 intergenic splice variant”.

According to this preferred embodiment there is provided a novel nucleic acid molecule in isolated form wherein said nucleic acid molecule comprises a DEC-205/DCL-1 intergenic splice variant.

Reference to “DEC-205” should be understood as a reference to a molecule of the family of type I transmembrane C-type lectin receptors that are, inter alia, expressed by dendritic cells. Reference to “DCL-1” is hereinafter defined.

The present invention still more particularly provides a nucleic acid molecule or derivative, homologue or analogue thereof comprising a nucleotide sequence encoding or a sequence complementary to a nucleotide sequence encoding an amino acid sequence substantially as set forth in SEQ ID NO:2 or SEQ ID NO:21 or a derivative, homologue or mimetic thereof having at least about 45% or greater similarity to at least 30 contiguous amino acids in SEQ ID NO:2 or SEQ ID NO:21.

The term “similarity” as used herein includes exact identity between compared sequences at the nucleotide or amino acid levels. Where there is non-identity at the nucleotide level “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. The percentage similarity may be greater than 45% such as at least 50% or at least 55% or at least 60% or at least 65% or at least 70% or at least 75% or at least 80% or at least 85% or at least 90%, 91%, 92%, 93%, 94%, 95%, 96%, 97%, 98%, 99% or higher. To determine the percent identity of two amino acid sequences or of two nucleic acids, the sequences may be aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions can then be compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e. % identity=# of identical positions/total # of overlapping positions×100). Preferably, the two sequences are the same length. The determination of percent identity or homology between two sequences can be accomplished using a mathematical algorithm. A suitable, mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to the nucleic acid molecules of the invention. BLAST protein searches can be performed with XBLAST program, score=50, wordlength=3 to obtain amino acid sequences homologous to the protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, only exact matches are counted. Yet another example of a suitable algorithm is one such Gap which considers all possible alignment and gap positions and creates an alignment with the largest number of matches bases and the fewest gaps. Gap uses the alignment method of Needleman and Wunsch. Gap reads a scoring matrix that contains values for ever possible GCG symbol match. GAP is available on ANGIS (Australian National Genomic Information Service) at website http://mell.angis.org.au.

In another embodiment, the present invention provides a novel nucleic acid molecule or a derivative, homologue or analogue thereof in isolated form comprising a nucleotide sequence or a sequence complementary thereto substantially as set forth in SEQ ID NO:1 or SEQ ID NO:20 or a nucleotide sequence having at least about 50% similarity to all or part thereof or a nucleotide sequence capable of hybridising to the sequence set forth in SEQ ID NO:1 or SEQ ID NO:20 under low stringency conditions at 42° C.

Preferably, the present invention contemplates a nucleic acid molecule or derivative, homologue or analogue thereof comprising a nucleotide sequence or a sequence complementary thereto substantially as set forth in SEQ ID NO:1 or SEQ ID NO:20 or a derivative thereof or capable of hybridising to SEQ ID NO:1 or SEQ ID NO:20 under low stringency conditions at 42° C. and which encodes an amino acid sequence corresponding to an amino acid sequence set forth in SEQ ID NO:2 or SEQ ID NO:21 or a sequence having at least about 45% similarity to at least 10 contiguous amino acids in SEQ ID NO:2 or SEQ ID NO:21.

More particularly, the present invention contemplates a nucleic acid molecule comprising a sequence of nucleotides substantially as set forth in SEQ ID NO:1 or SEQ ID NO:20.

Reference herein to a low stringency includes and encompasses from at least about 0% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0. 15M salt for hybridisation, and at least about 0.01M to at least about 0.15M salt for washing conditions. Stringency may be measured using a range of temperature such as from about 40° C. to about 65° C. Particularly useful stringency conditions are at 42° C. In general, washing is carried out at Tₘ=69.3+0.41 (G+C)%=−12° C. However, the Tₘ of a duplex DNA decreases by 1° C. with every increase of 1% in the number of mismatched based pairs (Bonner et al (1973) J. Mol. Biol., 81:123).

The nucleic acid molecule according to this aspect of the present invention corresponds herein to “DEC-205 SV”. Reference to the expression product appears in non-italicised text. Without limiting the present invention to any one theory or mode of action, it has been determined that DEC-205 SV mRNA encodes the full ectodomain of DEC-205 together with the carbohydrate recognition domain, transmembrane and cytoplasmic domain of DCL-1. The ectodomain of DEC-205 comprises a signal peptide, cysteine rich domain, fibronectin type II domain and 10 lectin-like carbohydrate recognition domains. The junction of DEC-205/DCL-1 mRNA is in frame, indicating that DEC-205 SV mRNA can be translated successfully. Both the DEC-205 and DCL-1 genes map to chromosome 2q24 and consist of 35 and 6 exons, respectively. These genes are separated by 5.4 kb. As detailed hereinbefore, the DCL-1 gene is a novel gene which has been identified by the inventors in respect of the present invention. More detailed discussion in relation to DCL-1 is provided hereinafter.

In one embodiment a DEC-205 SV mRNA is thought to be generated by transcribing a cistronic mRNA containing DEC-205 and DCL-1 gene followed by splicing out of DEC-205 exon 35 and DCL exon 1 (herein referred to as the “DEC-205 SV34”). In another embodiment, another DEC-205 SV mRNA is generated by transcribing a cistronic mRNA containing DEC-205 and DCL-1 gene followed by splicing out of DEC-205 exons 34 and 35, together with DCL-1 exon 1. Accordingly, there occurs fusion of the DEC-205 exon 33 to DCL-1 exon 2 (herein referred to as the “DEC-205 SV33”). The generation of DEC-205 SV therefore involves an intergenic splicing event, being an extremely rare event. The inventors have determined that the 5′ proximal promoter regions for DEC-205 and DCL-1 show independent promoter activity, thereby confirming their status as independent genes. This further confirms that the generation of DEC-205 SV clearly involves an intergenic splicing event.

The human DEC-205 SV34 expression product is defined by the amino acid sequence set forth in SEQ ID NO:2 while the DEC-205 SV33 expression product is defined by the amino acid sequence set forth in SEQ ID NO:21. The cDNA nucleotide sequence for human DEC-205 SV34 is set forth in SEQ ID NO:1 and the cDNA nucleotide sequence for human DEC-205 SV33 is set forth in SEQ ID NO:20. The nucleic acid molecules encoding the DEC-205 SV expression products are preferably a sequence of deoxyribonucleic acids such as a cDNA sequence or a genomic sequence. A cDNA sequence may optionally comprise all or some of the 5′ or 3′ untranslated regions while a genomic sequence may also comprise introns. A genomic sequence may also include a promoter region or other regulatory regions. It should also be understood that the subject nucleic acid molecule may be a sequence of ribonucleic acids such as mRNA.

In a particularly preferred embodiment, the present invention provides a novel cDNA or a derivative, homologue or analogue thereof or a sequence complementary thereto in isolated form comprising a nucleotide sequence substantially as set forth in SEQ ID NO:1 or SEQ ID NO:20 or a nucleotide sequence having at least about 50% similarity to all or part thereof or a nucleotide sequence capable of hybridising to the sequence set forth in SEQ ID NO:1 or SEQ ID NO:20 under low stringency conditions at 42° C.

As detailed hereinbefore, the present invention extends to nucleic acid molecules complementary to DEC-205 SV. In this regard, two examples of such complementary nucleic acid molecules are the nucleic acid molecules provided in SEQ ID NO:3 and SEQ ID NO:22 which are complementary to SEQ ID NO:1 and SEQ ID NO:20, respectively.

In a related aspect, the inventors have determined that the DCL-1 gene with which the DEC-205 is intergenically spliced to create the novel splice variants of the present invention is, itself, a novel gene. Specifically, it has been determined that DCL-1 corresponds to a unique type I transmembrane C-type lectin, the ectodomain of which contains only one CRD, whereas other type I transmembrane C-type lectins contain more than one domain. The DCL-1 expression product contains several putative motifs including a Tyr-based internalisation, a cluster of acidic amino acids and Ser- and Tyr-phosphorylation motifs. Without limiting the present invention to any one theory or mode of action, these features suggest that DCL-1 mediates not only endocytosis and late endosome targeting but also signalling.

Accordingly, another aspect of the present invention provides a nucleic acid molecule or derivative, homologue or analogue thereof comprising a nucleotide sequence encoding or a sequence complementary to a nucleotide sequence encoding an amino acid sequence substantially as set forth in SEQ ID NO:5 or a derivative, homologue or mimetic thereof having at least about 45% or greater similarity to at least 30 contiguous amino acids in SEQ ID NO:5.

In another aspect there is provided a nucleic acid molecule or derivative, homologue or analogue thereof comprising a nucleotide sequence encoding or a sequence complementary to a nucleotide sequence encoding an amino acid sequence substantially as set forth in SEQ ID NO:8 or a derivative, homologue or mimetic thereof having at least about 45% or greater similarity to at least 30 contiguous amino acids in SEQ ID NO:8.

In still another aspect there is provided a nucleic acid molecule or derivative, homologue or analogue thereof comprising a nucleotide sequence encoding or a sequence complementary to a nucleotide sequence encoding an amino acid sequence substantially as set forth in SEQ ID NO:11 or a derivative, homologue or mimetic thereof having at least about 45% or greater similarity to at least 30 contiguous amino acids in SEQ ID NO:11.

Reference to “similarity” should have the same meaning as hereinbefore provided.

In another embodiment, the present invention provides a novel nucleic acid molecule or a derivative, homologue or analogue thereof in isolated form comprising a nucleotide sequence or a sequence complementary thereto substantially as set forth in SEQ ID NO:4 or a nucleotide sequence having at least about 50% similarity to all or part thereof or a nucleotide sequence capable of hybridising to the sequence set forth in SEQ ID NO:4 under low stringency conditions at 42° C.

In still another embodiment, the present invention provides a novel nucleic acid molecule or a derivative, homologue or analogue thereof in isolated form comprising a nucleotide sequence or a sequence complementary thereto substantially as set forth in SEQ ID NO:32 or a nucleotide sequence having at least about 50% similarity to all or part thereof or a nucleotide sequence capable of hybridising to the sequence set forth in SEQ ID NO:32 under low stringency conditions at 42° C.

In yet another embodiment, the present invention provides a novel nucleic acid molecule or a derivative, homologue or analogue thereof in isolated form comprising a nucleotide sequence or a sequence complementary thereto substantially as set forth in SEQ ID NO:7 or a nucleotide sequence having at least about 50% similarity to all or part thereof or a nucleotide sequence capable of hybridising to the sequence set forth in SEQ ID NO:7 under low stringency conditions at 42° C.

In still another embodiment, the present invention provides a novel nucleic acid molecule or a derivative, homologue or analogue thereof in isolated form comprising a nucleotide sequence or a sequence complementary thereto substantially as set forth in SEQ ID NO:10 or a nucleotide sequence having at least about 50% similarity to all or part thereof or a nucleotide sequence capable of hybridising to the sequence set forth in SEQ ID NO:10 under low stringency conditions at 42° C.

Preferably, the present invention contemplates a nucleic acid molecule or derivative, homologue or analogue thereof comprising a nucleotide sequence or a sequence complementary thereto substantially as set forth in SEQ ID NO:4 or a derivative thereof capable of hybridising to SEQ ID NO:4 under low stringency conditions at 42° C. and which encodes an amino acid sequence corresponding to an amino acid sequence set forth in SEQ ID NO:5 or a sequence having at least about 45% similarity to at least 30 contiguous amino acids in SEQ ID NO:5.

In another preferred embodiment, the present invention contemplates a nucleic acid molecule or derivative, homologue or analogue thereof comprising a nucleotide sequence or a sequence complementary thereto substantially as set forth in SEQ ID NO:32 or a derivative thereof capable of hybridising to SEQ ID NO:32 under low stringency conditions at 42° C. and which encodes an amino acid sequence corresponding to an amino acid sequence set forth in SEQ ID NO:32 or a sequence having at least about 45% similarity to at least 30 contiguous amino acids in SEQ ID NO:32.

In yet another preferred embodiment, the present invention contemplates a nucleic acid molecule or derivative, homologue or analogue thereof comprising a nucleotide sequence or a sequence complementary thereto substantially as set forth in SEQ ID NO:7 or a derivative thereof capable of hybridising to SEQ ID NO:7 under low stringency conditions at 42° C. and which encodes an amino acid sequence corresponding to an amino acid sequence set forth in SEQ ID NO:8 or a sequence having at least about 45% similarity to at least 30 contiguous amino acids in SEQ ID NO:8.

In still another preferred embodiment, the present invention contemplates a nucleic acid molecule or derivative, homologue or analogue thereof comprising a nucleotide sequence or a sequence complementary thereto substantially as set forth in SEQ ID NO:10 or a derivative thereof capable of hybridising to SEQ ID NO:10 under low stringency conditions at 42° C. and which encodes an amino acid sequence corresponding to an amino acid sequence set forth in SEQ ID NO:11 or a sequence having at least about 45% similarity to at least 30 contiguous amino acids in SEQ ID NO:11.

Most particularly, the present invention contemplates a nucleic acid molecule comprising a sequence of nucleotides substantially as set forth in SEQ ID NO:4, SEQ ID NO:7 or SEQ ID NO:10 or SEQ ID NO:32.

Reference to “stringency” should have the same meaning as hereinbefore provided.

The nucleic acid molecule according to this aspect of the present invention corresponds herein to “DCL-1”. This gene has been determined in accordance with the present invention to encode a novel type I transmembrane C-type lectin which encodes only one CRD. The product of the DCL-1 gene is referred to herein as “DCL-1” (non-italicised text). DCL-1 is a protein for which intergenic splice variants exist, thereby resulting in the expression of a variety of intergenic isoforms. These have been hereinbefore described and are encompassed by the scope of the present invention. Further, a number of homologues of DCL-1 have been identified and described herein. Human DCL-1 is defined by the amino acid sequence set forth in SEQ ID NO:5, murine DCL-1 is defined by the amino acid sequence set forth in SEQ ID NO:8 and rat DCL-1 is defined by the amino acid sequence set forth in SEQ ID NO:11. The cDNA and genomic nucleotide sequences for human DCL-1 are defined by the nucleotide sequences set forth in SEQ ID NO:4. Murine and rat cDNA DCL-1 sequences are defined by the nucleotide sequences set forth in SEQ ID NO:7 and 10, respectively. SEQ ID NO:13 discloses a partial sequence of bovine DCL-1. As detailed hereinbefore, the nucleic acid molecules encoding DCL-1 expression products are preferably a sequence of deoxyribonucleic acids such as cDNA sequences or a genomic sequence. A cDNA sequence may optionally comprise all or some of the 5′ or 3′ untranslated regions while a genomic sequence may also comprise introns. A genomic sequence may also include a promoter region or other regulatory regions. It should also be understood that the subject nucleic acid molecules may be a sequence of ribonucleic acids such as mRNA.

The present invention extends to nucleic acid molecules complementary to DCL-1. In this regard, examples of such complementary nucleic acid molecules are the nucleic acid molecules provided in SEQ ID NOs:6, 9 and 12 which are complementary to SEQ ID NOs:4, 7 and 10, respectively.

The nucleic acid molecule of the present invention is preferably in isolated form or ligated to a vector, such as an expression vector. By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure.

The nucleic acid molecule may be ligated to an expression vector capable of expression in a prokaryotic cell (e.g. E.coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector, The latter embodiment facilitates production of recombinant forms of DEC-205 SV or DCL-1 which forms are encompassed by the present invention.

The expression product of the splice variant disclosed herein is a novel DEC-205 intergenic splice variant having an amino acid sequence set forth in SEQ ID NO:2 or SEQ ID NO:21 or is a derivative, homologue, analogue, chemical equivalent or mimetic thereof or is a molecule having an amino acid sequence of at least about 45% similarity to at least 30 contiguous amino acids in the amino acid sequence as set forth in SEQ ID NO:2 or SEQ ID NO:21 or a derivative, homologue, analogue, chemical equivalent or mimetic thereof.

The expression product of the novel lectin molecule disclosed herein is a novel DCL-1 molecule having an amino acid sequence set forth in SEQ ID NOs:5, 8 or 11 or is a derivative, homologue, analogue, chemical equivalent or mimetic thereof or is a molecule having an amino acid sequence of at least about 45% similarity to at least 30 contiguous amino acids in the amino acid sequence set forth in SEQ ID NO:5, 8 or 11, respectively or a derivative, homologue, analogue, chemical equivalent or mimetic thereof.

Accordingly, another aspect of the present invention is directed to a isolated protein selected from the list consisting of:
(ii) An isolated DEC-205 intergenic splice variant or a derivative, homologue, analogue, chemical equivalent or mimetic thereof.
(ii) An isolated DEC-205/DCL-1 intergenic splice variant or a derivative, homologue, analogue, chemical equivalent or mimetic thereof.
(xv) A protein having an amino acid sequence substantially as set forth in SEQ ID NO:2 or SEQ ID NO:21 or a derivative, homologue or mimetic thereof or a sequence having at least about 45% similarity to at least 30 contiguous amino acids in SEQ ID NO:2 or SEQ ID NO:21 or a derivative, homologue, analogue, chemical equivalent or mimetic of said protein.
(xvi) A protein encoded by a nucleotide sequence substantially as set forth in SEQ ID NO:1 or SEQ ID NO:20 or a derivative, homologue or analogue of said nucleotide sequence or a derivative, homologue, analogue, chemical equivalent or mimetic of said protein.
(xvii) A protein encoded by a nucleotide sequence substantially as set forth in SEQ ID NO:1 or SEQ ID NO:20 or a derivative, homologue or analogue thereof or a sequence encoding an amino acid sequence having at least about 45% similarity to at least 30 contiguous amino acids in SEQ ID NO:2 or SEQ ID NO:21 or a derivative, homologue, analogue, chemical equivalent or mimetic of said protein.
(xviii) A protein encoded by a nucleic acid molecule capable of hybridising to the nucleotide sequence set forth in SEQ ID NO:1 or SEQ ID NO:20 or a derivative, homologue or analogue thereof under low stringency conditions at 42° C. or a derivative, homologue, analogue, chemical equivalent or mimetic of said protein.
(xix) A protein encoded by a nucleic acid molecule capable of hybridising to the nucleotide sequence as set forth in SEQ ID NO:1 or SEQ ID NO:20 or a derivative, homologue or analogue thereof under low stringency conditions at 42° C. and which encodes an amino acid sequence substantially as set forth in SEQ ID NO:2 or SEQ ID NO:21 or a derivative, homologue or mimetic thereof or an amino acid sequence having at least about 45% similarity to at least 30 contiguous amino acids in SEQ ID NO:2 or SEQ ID NO:21.
(xx) A protein having an amino acid sequence substantially as set forth in SEQ ID NO:5, SEQ ID NO:8, or SEQ ID NO:11 or a derivative, homologue or mimetic thereof or a sequence having at least about 45% similarity to at least 30 contiguous amino acids in SEQ ID NO:5, SEQ ID NO: 8, or SEQ ID NO:11 or a derivative, homologue, analogue, chemical equivalent or mimetic of said protein.
(xxi) A protein encoded by a nucleotide sequence substantially as set forth in SEQ ID NOs:4, 7 or 10 or a derivative, homologue or analogue of said nucleotide sequence or a derivative, homologue, analogue, chemical equivalent or mimetic of said protein.
(xxii) A protein encoded by a nucleotide sequence substantially as set forth in SEQ ID NOs:4, 7 of 10 or a derivative, homologue or analogue thereof or a sequence encoding an amino acid sequence having at least about 45% similarity to at least 30 contiguous amino acids in SEQ ID NOs:5, 8 or 11 or a derivative, homologue, analogue, chemical equivalent or mimetic of said protein.
(xxiii) A protein encoded by a nucleic acid molecule capable of hybridising to the nucleotide sequence set forth in SEQ ID NOs:4, 7 or 10 or a derivative, homologue or analogue thereof under low stringency conditions at 42° C. or a derivative, homologue, analogue, chemical equivalent or mimetic of said protein
(xxiv) A protein encoded by a nucleic acid molecule capable of hybridising to the nucleotide sequence as set forth in SEQ ID NOs:4, 7 or 10 or a derivative, homologue or analogue thereof under low stringency conditions at 42° C. and which encodes an amino acid sequence substantially as set forth in SEQ ID NOs:5, 8 or 11 or a derivative, homologue or mimetic thereof or an amino acid sequence having at least about 45% similarity to at least 30 contiguous amino acids in SEQ ID NOs:5, 8 or 11.
(xxv) A protein as defined in any one of paragraphs (i) to (xii) in a homodimeric form.
(xxvi) A protein as defined in any one of paragraphs (i) to (xii) in a heterodimeric form.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater of subject protein relative to other components as determined by molecular weight, amino acid sequence or other convenient means. The protein of the present invention may also be considered, in a preferred embodiment, to be biologically pure.

The DEC-205 SV or DCL-1 of the present invention may be in multimeric form meaning that two or more molecules are associated together. Where the same DEC-205 SV or DCL-1 molecules are associated together, the complex is a homomultimer. An example of a homomultimer is a homodimer. Where at least one DEC-205 SV or DCL-1 is associated with at least one non-DEC-205 SV or DCL-1 molecule, then the complex is a heteromultimer such as a heterodimer.

The ability to produce recombinant DEC-205 SV or DCL-1 permits the large scale production of these molecules for commercial use. The DEC-205 SV or DCL-1 may need to be produced as part of a large peptide, polypeptide or protein which may be used as is or may first need to be processed in order to remove the extraneous proteinaceous sequences. Such processing includes digestion with proteases, peptidases and amidases or a range of chemical, electrochemical, sonic or mechanical disruption techniques.

Notwithstanding that the present invention encompasses recombinant proteins, chemical synthetic techniques are also preferred in synthesis of DEC-205 SV or DCL-1.

DEC-205 SV or DCL-1 according to the present invention is conveniently synthesised based on molecules isolated from a mammal. Isolation of these molecules may be accomplished by any suitable means such as by chromotographic separation, for example using CM-cellulose ion exchange chromatography followed by Sephadex (e.g. G-50 column) filtration. Many other techniques are available including HPLC, PAGE amongst others.

DEC-205 SV or DCL-1 may be synthesised by solid phase synthesis using F-moc chemistry as described by Carpino et al. (1991). DEC-205 SV and fragments thereof may also be synthesised by alternative chemistries including, but not limited to, t-Boc chemistry as described in Stewart et al. (1985) or by classical methods of liquid phase peptide synthesis.

The protein and/or gene is preferably from a human, primate, livestock animal (e.g. sheep, pig, cow, horse, donkey), laboratory test animal (e.g. mouse, rabbit, rat, guinea pig), companion animal (e.g. dog, cat), captive wild animal (e.g. fox, kangaroo, deer), aves (e.g. chicken, geese, duck, emu, ostrich), reptile or fish. Most preferably, the gene is of human or primate origin.

Without limiting the present invention to any one theory or mode of action, genes encoding DEC-205 and DCL-1 are juxtaposed within chromosome band 2q24 and are separated by only approximately 5.4 kb. These two genes are independent genes because both DEC-205 and DLC-1 mRNA are expressed independently in haematopoietic cell lines. Further, luciferase reporter assay studies show that both the 5′-proximal promoters of DEC-205 and DCL-1 have independent promoter activities. Still without limiting the invention in any way, all Hodgkin and Reed-Sternberg cells express the 9.5 kb DEC-205 SV mRNA indicating that expression of this mRNA is highly regulated. Accordingly, it is thought that mechanisms which transcriptionally control expression of this splice variant molecule may be involved in the pathogenesis of Hodgkin's disease. Still further, the presence of this molecule in classical Hodgkin's lymphoma provides a target for antibody or T-cell mediated immunotherapy for this disease condition.

The present invention therefore contemplates a method of modulating DEC-205 SV expression or DEC-205 SV functional activity in a mammal, said method comprising administering to said mammal an agent for a time and under conditions sufficient to up-regulate, down-regulate or otherwise modulate expression of DEC-205 SV or functioning of DEC-205 SV.

For example, DEC-205 SV antisense sequences such as oligonucleotides may be introduced into a cell to down-regulate the expression of DEC-205/DCL-1. Conversely, a nucleic acid molecule encoding DEC-205/DCL-1 or a derivative thereof may be introduced to enhance the functioning of DEC-205 SV in any cell expressing the endogenous DEC-205 SV gene. Although the preferred method is to down-regulate the expression of this molecule as a means for therapeutically or prophylactically treating Hodgkin's lymphoma, it should be understood that the present invention also extends to up-regulation of the expression of this molecule which may be desired in certain circumstances, such as for the purpose of creating cell lines for further studies.

Reference to “DEC-205 SV” should be understood as a reference to all splice variant forms of this molecule including, for example, the DEC-205 SV34 and DEC-205 SV33 forms of this splice variant.

In accordance with the other aspect of the present invention, and without limiting this aspect of the present invention in any way, as detailed hereinbefore DCL-1 is a unique type I transmembrane C-type lectin which expresses an ectodomain containing only one CRD. Most other type I transmembrane C-type lectins contain more than one domain. It is thought that since DCL-1 comprises putative motifs including a Tyr based internalisation, a cluster of acidic amino acids and Ser- and Tyr-phosphorylation motifs, that DCL-1 mediates not only endocytosis and late endosome targeting but also signalling. Further, it has been found that this molecule is expressed in myeloid and B cells.

Accordingly, another aspect of the present invention is directed to a method for modulating DCL-1 expression or DCL-1 functional activity in a mammal, said method comprising administering to said mammal an agent for a time and under conditions sufficient to up-regulate, down-regulate or otherwise modulate said expression or functioning.

The cloning and sequencing of these molecules and their expression products now provides a mechanism for both the development of diagnosis/prognosis methodology and the prophylactic and therapeutic treatment of conditions such as Hodgkin's lymphoma. Accordingly, the present invention contemplates therapeutic, prophylactic, diagnostic and prognostic uses of DEC-205 SV amino acid and nucleic acid molecules, DCL-1 amino acid and nucleic acid molecules and agonistic and antagonistic agents thereto, for the regulation of cell functional activity.

The present invention contemplates, therefore, a method for regulating cellular activity in a subject said method comprising administering to said subject an effective amount of an agent for a time and under conditions sufficient to modulate DEC-205 SV expression of DEC-205 SV functional activity.

In yet another aspect there is contemplated a method of regulating cellular activity in a subject said method comprising administering to said subject an effective amount of an agent for a time and conditions sufficient to modulate DCL-1 expression or DCL-1 functional activity.

Reference to “cellular activity” should be understood as a reference to one or more of the functional activities which are directly or indirectly regulated via the DEC-205 SV or DCL-1 expression products. This includes, but is not limited to, cellular endocytosis, late endosome targeting, signalling (in respect of the DCL-1 molecule), Hodgkin and Reed-Sternberg cell functioning (in respect of the DEC-205 SV molecule) and antigen presenting cell antigen uptake (in respect of the DCL-1 molecule).

In terms of achieving the up or down-regulation of DEC-205 SV or DCL-1 expression or functioning, means for achieving this objective would be well known to the person of skill in the art and include, but are not limited to:
(i) Introducing into a cell a nucleic acid molecule encoding DEC-205 SV or DCL-1 or functional equivalent, derivative or analogue thereof in order to up-regulate the capacity of said cell to express DEC-205 SV or DCL-1, respectively.
(ii) Introducing into a cell a proteinaceous or non-proteinaceous molecule which modulates transcriptional and/or translational regulation of a gene, wherein this gene may be DEC-205 SV or DCL-1 or functional portion thereof or some other gene which directly or indirectly modulates the expression of DEC-205 SV or DCL-1.
(iii) Introducing a proteinaceous or non-proteinaceous molecule which functions as an antagonist to the DEC-205 SV or DCL-1 expression product.
(iv) Introducing a proteinaceous or non-proteinaceous molecule which functions as an agonist of the DEC-205 SV or DCL-1 expression product (this should be understood to extend to administering the DEC-205 SV or DCL-1 expression product).

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the DEC-205 SV or DCL-1 expression product or small molecules capable of acting as agonists or antagonists. Chemical agonists may not necessarily be derived from the DEC-205 SV or DCL-1 expression product but may share certain conformational similarities. Alternatively, chemical agonists may be specifically designed to meet certain physiochemical properties. Antagonists may be any compound capable of blocking, inhibiting or otherwise preventing DEC-205 SV or DCL-1 from carrying out its normal biological function. Antagonists include monoclonal antibodies and antisense nucleic acids which prevent transcription or translation of DEC-205 SV or DCL-1 genes or mRNA in mammalian cells. Modulation of expression may also be achieved utilising antigens, RNA, ribosomes, DNAzymes, RNA aptamers, antibodies or molecules suitable for use in cosuppression. The proteinaceous and non-proteinaceous molecules referred to in points (i)-(iv), above, are herein collectively referred to as “modulatory agents”.

Screening for the modulatory agents hereinbefore defined can be achieved by any one of several suitable methods including, but in no way limited to, contacting a cell comprising the DEC-205 SV or DCL-1 gene or functional equivalent or derivative thereof with an agent and screening for the modulation of DEC-205 SV or DCL-1 protein production or functional activity, modulation of the expression of a nucleic acid molecule encoding DEC-205 SV or DCL-1 or modulation of the activity or expression of a downstream functional activity. Detecting such modulation can be achieved utilising techniques such as Western blotting, electrophoretic mobility shift assays and/or the readout of reporter genes

The present invention should be understood to extend to methods of screening for such agents.

It should be understood that the DEC-205 SV or DCL-1 gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate DEC-205 SV or DCL-1 activity, at either the nucleic acid or expression product levels, or the gene may require activation—thereby providing a model useful for, inter alia, screening for agents which up regulate DEC-205 SV or DCL-1 expression. Further, to the extent that a DEC-205 SV or DCL-1 nucleic acid molecule is transfected into a cell, that molecule may comprise the entire DEC-205 SV or DCL-1 gene or it may merely comprise a portion of the gene such as the portion which regulates expression of the DEC-205 SV or DCL-1 product. For example, the DEC-205 SV or DCL-1 promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream DEC-205 SV or DCL-1 regulatory target, rather than DEC-205 SV or DCL-1 itself. Yet another example includes DEC-205 SV or DCL-1 binding sites ligated to a minimal reporter, For example, modulation of DEC-205 SV or DCL-1 activity can be detected by screening for the modulation of the functional activity in a Hodgkin and Reed-Sternberg cell or other suitable cell. This is an example of an indirect system where modulation of DEC-205 SV or DCL-1 expression, per se, is not the subject of detection.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the DEC-205 SV or DCL-1 nucleic acid molecule or expression product itself or which modulate the expression of an upstream molecule, which upstream molecule subsequently modulates DEC-205 SV or DCL-1 expression or expression product activity. Accordingly, these methods provide a mechanism for detecting agents which either directly or indirectly modulate DEC-205 SV or DCL-1 expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region.

The subject proteinaceous or non-proteinaceous molecule may act either directly or indirectly to modulate the expression of DEC-205 SV or DCL-1 or the activity of the DEC-205 SV or DCL-1 expression product. Said molecule acts directly if it associates with the DEC-205 SV or DCL-1 nucleic acid molecule or expression product to modulate expression or activity, respectively. Said molecule acts indirectly if it associates with a molecule other than the DEC-205 SV or DCL-1 nucleic acid molecule or expression product which other molecule either directly or indirectly modulates the expression or activity of the DEC-205 SV or DCL-1 nucleic acid molecule or expression product, respectively. Accordingly, the method of the present invention encompasses the regulation of DEC-205 SV or DCL-1 nucleic acid molecule expression or expression product activity via the induction of a cascade of regulatory steps.

The term “expression” in this context refers to the transcription and translation of a nucleic acid molecule. Reference to “expression product” is a reference to the product produced from the transcription and translation of a nucleic acid molecule.

“Derivatives” of the molecules herein described (for example DEC-205 SV or DCL-1 or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, DEC-205 SV or DCL-1 or derivative thereof may be fused to a molecule to facilitate its homing to a cell. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” of DEC-205 SV or DCL-1 should be understood to mean molecules which exhibit at least some of the functional activity of the form of DEC-205 SV or DCL-1 of which it is a variant. A variation may take any form and may be naturally or non-naturally occurring. A mutant molecule is one which exhibits modified functional activity.

By “homologue” is meant a molecule derived from a species other than human.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing analogues which exhibit properties such as more potent pharmacological effects. DEC-205 SV or DCL-1 or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of DEC-205 SV or DCL-1, it may also be desirable to identify and utilise molecules which function agonistically or antagonistically to DEC-205 SV or DCL-1 in order to up or down-regulate the functional activity of DEC-205 SV or DCL-1 in relation to modulating cell functioning. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of DEC-205 SV or DCL-1 capable of acting as agonists or antagonists. Chemical agonists may not necessarily be derived from DEC-205 SV or DCL-1 but may share certain conformational similarities. Alternatively, chemical agonists may be specifically designed to mimic certain physiochemical properties of DEC-205 SV or DCL-1. Antagonists may be any compound capable of blocking, inhibiting or otherwise preventing DEC-205 SV or DCL-1 from carrying out its normal biological functions. Antagonists include monoclonal antibodies specific for DEC-205 SV or DCL-1 or parts of DEC-205 SV or DCL-1.

Analogues of DEC-205 SV or DCL-1 or of DEC-205 SV or DCL-1 agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH4.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional amino acid | Code |
|---|---|
| α-aminobutyric acid | Abu |
| α-amino-α-methylbutyrate | Mgabu |
| aminocyclopropane-carboxylate | Cpro |
| aminoisobutyric acid | Aib |
| aminonorbornyl-carboxylate | Norb |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl)carbamylmethyl)glycine | Nnbhm |
| 1-carboxy-1-(2,2-diphenyl-Nmbc ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl- -aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl)carbamylmethyl)glycine | Nnbhe |

Crosslinkers can be used, for example, to stabilise 3D conformations, using homo-bifuictional crosslinkers such as the bifunctional imido esters having (CH2)n spacer groups with n=1 to n=6, glutaraldehyde, N-hydroxysuccinimide esters and hetero-bifunctional reagents which usually contain an amino-reactive moiety.

These types of modifications may be important to stabilise the molecule if administered to an individual or for use as a diagnostic reagent.

The present invention further contemplates analogues capable of acting as antagonists or agonists of the native amino acid or nucleic acid molecules or which can act as functional analogues of the native molecules (herein referred to as an “antagonist” or an “agonist”). Analogues, antagonists and agonists may not necessarily be derived from the subject molecules but may share certain conformational similarities. Alternatively, analogues, antagonists and agonists may be specifically designed to mimic certain physiochemical properties of the molecules. Analogues, antagonists and agonists may be chemically synthesised or may be detected following, for example, natural product screening.

Derivatives also extend to fragments having particular epitopes or parts of the entire molecule fused to peptides, polypeptides or other proteins. The derivatives of the nucleic acid molecules of the present invention include oligonucleotides, PCR primers, antisense molecules, molecules suitable for use in cosuppression and fusion of nucleic acid molecules.

An “effective amount” means an amount necessary at least partly to attain the desired immune response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

It should be understood that the target cell which is treated according to the method of the present invention may be located ex vivo or in vivo. By “ex vivo” is meant that the cell has been removed from the body of a subject wherein the modulation of its activity will be achieved in vitro. In accordance with the preferred aspect of the present invention, the cell may be a neoplastic cell, such as a Hodgkin and Reed-Sternberg cell, located in vivo and the down-regulation of its growth will be achieved by applying the method of the present invention in vivo.

It should be understood that the reference to a “cell” in the context of the present invention is a reference to any form or type of cell, irrespective of its origin. For example, the cell may be a naturally occurring normal or abnormal cell or it may be manipulated, modified or otherwise treated either in vitro or in vivo such as a cell which has been freeze/thawed or genetically, biochemically or otherwise modified either in vitro or in vivo (including, for example, cells which are the result of the fusion of two distinct cell types).

A further aspect of the present invention relates to the use of the invention in relation to the treatment and/or prophylaxis of disease conditions characterised by aberrant, unwanted or inappropriate functioning of DEC-205 SV or DCL-1. Still further, the present invention is particularly useful, but in no way limited to, use in the treatment of Hodgkin's lymphoma which is characterised by the Hodgkin and Reed-Sternberg cells which express DEC-205 SV.

The present invention therefore contemplates a method for the treatment and/or prophylaxis of a condition characterised by aberrant, unwanted or otherwise inappropriate functioning of DEC-205 SV or DCL-1 in a subject, said method comprising administering to said subject an effective amount of an agent as hereinbefore defined for a time and under conditions sufficient to modulate the expression of DEC-205 SV or DCL-1 and/or functioning of DEC-205 SV or DCL-1.

Reference to “aberrant, unwanted or otherwise inappropriate” activity should be understood as a reference to overactivity, underactivity or to physiologically normal activity which is inappropriate in that it is unwanted.

In yet another aspect, the present invention provides a means of targeting a therapeutic treatment method to Hodgkin's lymphoma cells on the basis of their unique expression of the DEC-205 SV expression product. In particular, the unique expression of this molecule by the Hodgkin and Reed-Sternberg malignant cells provides a means for targeting therapeutic means such as immunological cytolytic means (eg. cytotoxic T cell or antibody) or cytotoxic means such as those characterised by the use of chemotherapeutic agents.

According to this aspect of the present invention there is provided a method for the treatment of Hodgkin's lymphoma in a mammal, said method comprising administering to said mammal an effective amount of a cytolytic and/or cytotoxic agent which agent interacts or otherwise associates with DEC-205 SV, for a time and under conditions sufficient for said agent to lyse, apoptose or otherwise kill Hodgkin and Reed-Sternberg cells.

In still another aspect, the inventors have determined that DCL-1 may be used as an antigen loading receptor for antigen presenting cells, such as dendritic cells, in the context of immunotherapy. Accordingly, the present invention should also be understood to be directed to methods of modulating the generation of an immune response to an antigen via modulation of the association of antigen presenting cell DCL-1 molecules with the subject antigen. Without limiting the present invention to any one theory or mode of action, it is thought that DCL-1 functions by binding and intemalising antigen such that it can be processed and re-expressed on the dendritic cell surface in a form suitable for presentation. Methods of agonising or antagonising the functioning of DCL-1 on antigen presenting cell surfaces, in particular dendritic cells, provides a means of either up- or down-regulating this process. Means of identifying agents suitable for use in this regard have been hereinbefore described in detail.

The subject of the treatment or prophylaxis is generally a mammal such as but not limited to human, primate, livestock animal (e.g. sheep, cow, horse, donkey, pig), companion animal (e.g. god, cat), laboratory test animal (e.g. mouse, rabbit, rat, guinea pig, hamster), captive wild animal (e.g. fox, deer). Preferably the mammal is a human or primate. Most preferably the mammal is a human. Although the present invention is exemplified using a murine model, this is not intended as a limitation on the application of the present invention to other species, in particular, human.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

Administration of the agent in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In another aspect, the present invention contemplates a pharmaceutical composition comprising a modulatory agent as hereinbefore defined and one or more pharmaceutically acceptable carriers and/or diluents. Said modulatory agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another aspect of the present invention relates to modulatory agents, as hereinbefore defined, when used in the method of the present invention.

Still another aspect of the present invention is directed to antibodies to DEC-205 SV or DCL-1 including catalytic antibodies. Such antibodies may be monoclonal or polyclonal and may be selected from naturally occurring antibodies to DEC-205 SV or DCL-1 or may be specifically raised to DEC-205 SV or DCL-1. In the case of the latter, DEC-205 SV or DCL-1 may first need to be associated with a carrier molecule. The antibodies and/or recombinant DEC-205 SV or DCL-1 of the present invention are particularly useful as therapeutic or diagnostic agents. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool for assessing apoptosis or monitoring the program of a therapeutic regime. For example, DEC-205 SV or DCL-1 can be used to screen for naturally occurring antibodies to DEC-205 SV.

In another example, specific antibodies can be used to screen for DEC-205 SV or DCL-1 proteins. The latter would be important, for example, as a means for screening for levels of DEC-205 SV or DCL-1 in a cell extract or other biological fluid or purifying DEC-205 SV or DCL-1 made by recombinant means from culture supernatant fluid. Techniques for the assays contemplated herein are known in the art and include, for example, sandwich assays, ELISA and flow cytometry.

Both polyclonal and monoclonal antibodies are obtainable by immunization with the protein or peptide derivatives and either type is utilizable for immunoassays. The methods of obtaining both types of sera are well known in the art. Polyclonal sera are less preferred but are relatively easily prepared by injection of a suitable laboratory animal with an effective amount of DEC-205 SV or DCL-1, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman, Basic Facts about Hybridomas, in Compendium of Immunology Vol II, ed. by Schwartz, 1981; Kohler and Milstein, Nature 256: 495499, 1975; European Journal of Immunology 6: 511-519, 1976).

In another aspect, the molecules of the present invention are also useful as screening targets for use in applications such as the diagnosis of disorders characterised by the expression of DEC-205 SV or DCL-1. For example, screening for the levels of DEC-205 SV protein or DEC-205 SV mRNA transcripts in tissues as an indicator of a predisposition to, or the development of, Hodgkin's lymphoma. More specifically, there is now provided a means for screening individuals for the presence of DEC-205 SV encoding nucleic acid molecules or expression product or the specific forms of DEC-205 SV which are transcribed and/or translated by a given population of cells. The screening methodology may be directed to qualitative and/or quantitative DEC-205 SV analysis.

Accordingly, yet another aspect of the present invention contemplates a method of monitoring a disease condition in a mammal, which disease condition is characterised by DEC-205 SV cellular expression, said method comprising screening for DEC-205 SV and/or DEC-205 SV in a biological sample isolated from said mammal.

Screening for DEC-205 SV or DEC-205 SV (or DCL-1 to the extent that it may prove to be a useful diagnostic marker) in a biological sample can be performed by any one of a number of suitable methods which are well known to those skilled in the art. Examples of suitable methods include, but are not limited to, in situ hybridisation of biopsy sections to detect mRNA transcript or DNA, Northern blotting, RT-PCR of specimens isolated from tissue biopsies or antibody screening of tissue sections.

To the extent that antibody based methods of diagnosis are used, the presence of DEC-205 SV or DEC-205 SV may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention the sample is one which might contain DEC-205 SV including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, a first antibody having specificity for the DEC-205 SV or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

Further features of the present invention are more fully described in the following non-limiting examples.

TABLE 4

| SEQ ID | |
|---|---|
| NO | SEQUENCE DESCRIPTION |
| <400>1 | Human DEC205/DCL-1 splice variant (exon 34 |
| | fusion): cDNA sequence |
| <400>2 | Human DEC205/DCL-1 splice variant (exon 34 |
| | fusion): amino acid sequence |
| <400>3 | Human DEC205/DCL-1 splice variant (exon 34 |
| | fusion): complementary DNA strand |
| <400>4 | Human DCL-1 cDNA sequence |
| <400>5 | Human DCL-1 amino acid sequence |
| <400>6 | Human DCL-1 complementary DNA sequence |
| <400>7 | Murine DCL-1 cDNA sequence |
| <400>8 | Murine DCL-1 amino acid sequence |
| <400>9 | Murine DCL-1 complementary DNA sequence |
| <400>10 | Rat DCL-1 cDNA sequence |
| <400>11 | Rat DCL-1 amino acid sequence |
| <400>12 | Rat DCL-1 complementary DNA sequence |
| <400>13 | Bovine DCL-1 EST sequence |
| <400>14 | FIG. 4 sequence |
| <400>15 | FIG. 4 sequence |
| <400>16 | FIG. 4 sequence |
| <400>17 | FIG. 4 sequence |
| <400>18 | FIG. 4 sequence |
| <400>19 | FIG. 4 sequence |
| <400>20 | Human DEC-205/DCL-1 cDNA (exon 33 fusion) |
| | sequence |
| <400>21 | Human DEC-205/DCL-1 amino acid (exon 33 |
| | fusion) sequence |
| <400>22 | Human DEC-205/DCL-1 (exon 33 fusion) |
| | complementary DNA strand sequence |
| <400>23 | Primer 62 |
| <400>24 | Primer 63 |
| <400>25 | Primer 78 |
| <400>26 | Primer 85 |
| <400>27 | Primer 86 |
| <400>28 | Primer 88 |
| <400>29 | Primer 90 |
| <400>30 | Primer 92 |
| <400>31 | Primer 94 |

### EXAMPLE 1

### Hodgkin's Lymphoma Cell Lines Express a Fusion Protein Encoded by Intergenically Splice mRNA for the Multilectin Receptor DEC-205 (CD205) and a Novel C-Type Lectin Receptor DCL-1

Materials and Methods

Cell Lines

The human hematopoietic cell lines, HEL, KG-1, K562, THP-1, U937, Mann, Daudi, Raji, WT49, Mann, Molt-4, Jurkat and HSB-2 were obtained from the American Type Culture Collection (Rockville, Md.). L428 cells were provided by V. Diehl (Klinik fur Innere Medizin, Cologne, Germany).²³ HDLM-2²⁴ and KM-H2 cells²⁵ were obtained from the German Collection of Microorganism and Cell Culture (Braunschweig, Germany). Mono Mac 6 cells²⁶ were provided by E. M. Schneider (Dusseldorf, Germany). All cell lines were maintained in RPMI 1640 (Invitrogen, Melbourne, VIC, Australia), 10% fetal calf serum (FCS, Invitrogen), 100 U/ml penicillin, and 100 μg/ml streptomycin, except for HDLM-2 cells, which were maintained in 20% FCS. These cells were subjected to RNA preparation using TRIzol (Invitrogen) for RT-PCR and Northern blot analysis.

Antibodies and other Reagents

The mAb MMRI-7 against human DEC-205 was produced in house.²⁷ MMRI-7 binds to an epitope within DEC-205 CRD 1 and 2. The other anti human DEC-205 mAb, M335 was provided through the 7^{th} International Workshop on Human Leukocyte Differentiation Antigens. M335 binds to an epitope within DEC-205 cysteine-rich domain (CR).²⁷

Goat anti mouse IgG was purchased from Dako (Botany, NSW, Australia). Horse radish peroxidase (HRP)-conjugated goat anti mouse IgG-Fc specific and protein A-conjugated agarose beads were from Sigma (Sydney, NSW, Australia). HRP-conjugated sheep anti rabbit IgG was from Silenus (Melbourne, VIC, Australia). ELISA plates (Maxsorb) were from Nalge Nunc International (Rochester, N.Y.). Prestained protein standards (Benchmark Prestained Protein Ladder) and DNA ladder (1 kb ladder) were from Invitrogen. Molecular biological enzymes (e.g. restriction enzymes, polymerases and ligase) were obtained from Invitrogen, Promega (Sydney, NSW, Australia) or Roche Applied Science (Castle Hill, NSW, Australia). Unless specified, general chemicals were obtained from Sigma (Castle Hill, NSW, Australia) or BDH (Poole, England).

Rabbit polyclonal peptide antisera against the DEC-205 CP domain and the DCL-1 CP were produced by immunizing New Zealand White rabbits with diphtheria toxoid-conjugated synthetic peptide CEDEIMLPSFHD and CGEENEYPYQFD (Minotopes, Clayton, VIC, Australia), respectively, using a conventional schedule with Freund adjuvant at the Herston Medical Research Institute (Herston, QLD, Australia). To assess the titer of the antibodies against CP peptides, an ELISA plate was coated with streptavidin (Sigma) and biotinylated peptides for DEC-205 CP (biotin-SGSGEDEIMLPSFHD) and DCL-1 CP (biotin-SGSGEENEYPYQFD) captured. The plate was blocked with 1% (w/v) sodium caseinate (Sigma) in PBS and 0.1% (w/v) Tween 20 (PBS/Tw), and incubated with serially diluted antisera. After washing the plate with PBS/Tw, bound antibody was detected with HRP-sheep anti rabbit IgG and o-phenylenediamine hydrochloride, and quantitated with 492 nm using an ELISA reader. There was no cross-reactivity detected between these two rabbit CP antibodies at the dilutions used in the experiments described (data not shown).

3′-Rapid Amplification of cDNA Ends (3′-RACE)

The 3′-end of DEC-205 mRNA was obtained by 3′-RACE was performed as described previously.¹⁷ Briefly, L428 mRNA was reverse transcribed with an oligo dT adaptor primer. The obtained L428 cDNA pool was subjected to PCR using DEC-205 specific forward primer and an adaptor primer, and cloned into pBlueScript SKII (Stratagene, La Jolla, Calif.). The clones analyzed by restriction enzyme mapping and sequencing using a BigDye Terminator kit on a ABI Prism 377 automated sequencer (PE Applied Biosystems, Scoresby, VIC, Australia) by Australian Genome Research Facility (University of Queensland, St. Lucia, QLD, Australia).

RT-PCR Analysis

PCR was performed on the L428 cDNA pool using DEC-205 specific forward primers (078, 088, 090, 092 and 094, nested within various parts of DEC-205 ectodomain) in combination with either DEC-205 specific reverse primer (085, nested within DEC-205 CP) or DCL-1 specific reverse primer (086, nested within DCL-1 ectodomain) with an Expand Long Template PCR system (Roche)(Table 3). The PCR reactions were fractionated in 0.8% agarose in Tris-acetate buffer (40 mM Tris-acetate, 1 mM EDTA, pH 7.6) and visualized with ethidium bromide. The PCR products obtained by the primer combination 078/085 and 078/086 were cloned into pGEM-T Easy vector (Promega) and sequenced.

Northern Blot Analysis

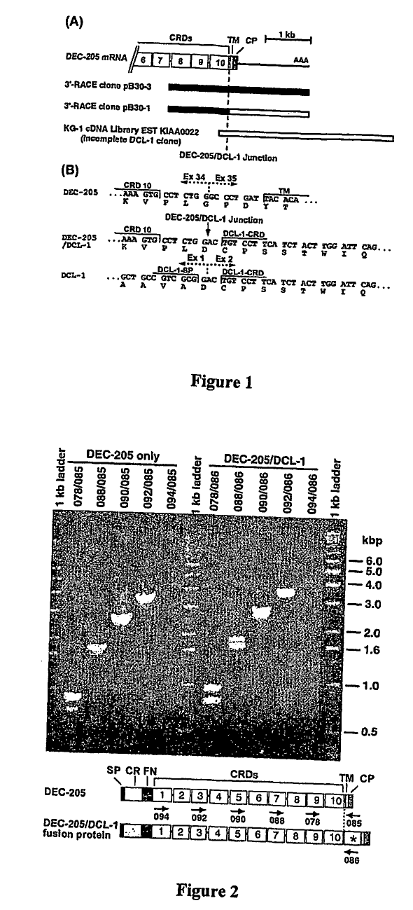

Approximately 10 μg of total RNA from cultured cell lines was fractionated in formaldehyde-denatured 1% agarose gel, and transferred to Hybond N⁺ cationic nylon membrane (Amersham Biosciences, Sydney, NSW, Australia). The 864 bp DEC-205 cDNA probe nested within DEC-205 CRD I and 2 was PCR amplified using primers 094 and 095 on the DEC-205 cDNA clone pCRD1/2-Ig²⁷ and Taq polymerase (Roche). The 1617 bp DCL-1 cDNA probe was PCR amplified using DCL-1 specific primers 062 and 063 on the pBS30-1 (FIG. 1). These probes were purified using QlAquick PCR Purification kit (Qiagen, Clifton Hill, VIC, Australia) and labeled with [α-³²P]dATP (Amersham Biosciences) using Strip-EZ DNA StipAble DNA probe Synthesis and Removal kit (Ambion, Austin, Tex.). The membrane was hybridized sequentially with these probes and exposed to a Kodak BioMax MS X-ray film at −70° C. using an intensifying screen (Amersham Biosciences). The final wash was 0.1×SSC (1×SSC is 0.15 M NaCl, 15 mM Na-citrate, pH7.0) and 0.5% SDS at 68° C. After each probing, the membrane was chemically stripped according to the manufacture's instruction, and used for hybridization with the other probes.

Preparation of Cell Lysate

Approximately 10⁷ cells were lysed with 1 ml of 0.15 M NaCl, 25 mM Tris-HCl, pH 7.4, 1% (v/v) Triton X-100, 0.5% (w/v) sodium deoxycholate, 0.1% (w/v) SDS and a cocktail of protease inhibitors (Complete, EDTA-free, Roche Applied Science) and incubated on ice for 10 min with occasional vortexing. After centrifugation at 12,000×g for 20 min at 4° C., the supernatant was collected and used directly for immunoprecipitation/Western blot or sandwich ELISA analysis described below.

Immunoprecipitation/Western Blot Analysis

The cell extract was precleared with a non-immune rabbit serum and protein A Sepharose (Sigma) for 1 h at 4° C., and subjected to immunoprecipitation using the rabbit peptide antisera against DEC−205 CP or DCL-1 CP with protein A Sepharose overnight at 4° C. The beads were washed with a wash buffer (0.15 M NaCl, 25 mM Tris-HCl, pH7.5, 0.2% (v/v) Triton X-100 and 0.5% (w/v) sodium deoxycholate), and eluted with SDS-PAGE sample buffer (2% (w/v) SDS, 62.5 mM Tris-HCl, pH6.8, 0.01% (w/v) bromophenol blue and 10% (v/v) glycerol) by heating at 95° C. for 5 min. The samples were subjected to Laemmli discontinuous SDS-PAGE with 10% (v/v) polyacrylamide separating gel²⁸ in the non-reducing condition, and transferred to a polyvinylidene fluoride membrane (PVDF-Plus, Osmonics, Westborough, Mass.). The membrane was blocked with 5% (w/v) non-fat dry milk in PBS/Tw (BLOTTO), incubated with a mixture of DEC-205 mAbs (MMRI-7 and M335, 5 μg/ml each) overnight at 4° C., and washed with PBS/Tw. The membrane was incubated with HRP-anti goat mouse IgG, and the bound enzyme was detected with enhanced chemiluminescence (SuperSignal West Pico, Pierce, Rockford, Ill.) on a Kodak X-Omat XB-1 X-ray film.

Sandwich ELISA

An ELISA plate was coated with 10 μg/ml goat anti mouse IgG in PBS, washed with PBS/Tw and blocked with BLOTTO. To the plate a mixture of DEC-205 mAb (MMRI-7 and M335, 2 μg/ml each) was added and incubated for 1 h at room temperature. The plate was washed and incubated with the serially diluted cell extracts overnight at 4° C. The plate was washed with PBS/Tw and incubated with either rabbit peptide antibodies against DEC-205 CP or DCL-1 CP (1:1000 dilution in PBS/Tw) or non immune rabbit serum for 1 h at room temperature and after washing with PBS/Tw, the plate was incubated with HRP-conjugated goat anti rabbit IgG in 5% mouse serum and PBS/TW. The plate was developed with o-phenylenediamine dihydrochloride and quantitated at 492 nm.

Results

Identification of the cDNA Clone Encoding DEC-205/DCL-1 Fusion

To obtain the 3′-end of human DEC-205 mRNA, we performed 3′-RACE.¹⁷ This resulted in amplification of an ˜3 kb PCR product (data not shown). When we cloned the PCR product and analyzed several clones by restriction enzyme analysis, however, we realized that there were two distinct sequences within the PCR product. The clone pB30-3 contained the authentic DEC-205 sequence encoding the DEC-205 CRD 8-10, TM and CP¹⁷. The other clone pB30-1, however, encoded DEC-205 CRD 8-10 followed by a unique sequence distinct from the DEC-205 TM and CP sequence (FIG. 1A). The junction of the DEC-205 and unique sequence was located within the connecting region (spacer 11) between the DEC-205 CRD10 and TM. A BLAST search identified the unique sequence as a part of the cDNA, KIAA0022 derived from KG-1 cell cDNA library²². Our further analysis showed that the KIAA0022 contained a partial cDNA encoding a novel type I transmembrane C-type lectin receptor, and we termed it, DCL-1 (DEC-205-associated C-type Lectin-1). The complete DCL-1 coding region encodes a signal peptide (SP), one CRD, one TM and one CP. DCL-1 was recently mapped to chromosome band 2q24. More details of DCL-1 will be published elsewhere (in preparation).

The sequence analysis showed that fusion junction occurred within the codon G/GC (/indicates the junction) for Gly in the DEC-205 spacer 11, connected to the codon G/AC for Asp in the junction between the DCL-1 SP and CRD. The fusion junction was in-frame, connecting the DEC-205 CRD 10 to the DCL-1 CRD, TM and CP, suggesting that the DEC-205/DCL-1 fusion mRNA is translated. Further, analysis of the DEC-205 and DCL-1 genes indicated that the junction is formed by splicing and fusing DEC-205 exon 34 to DCL-1 exon 2 (described below).

The DEC-205/DCL-1 Fusion mRNA Appears to Encode the Entire DEC-205 Ectodomain

We examined L428 cDNA containing the DEC-205/DCL-1 junction by RT-PCR to see whether it included the entire DEC-205 ectodomain (FIG. 2). The combination of the DEC-205 CP-specific reverse primer 085 with DEC-205-specific forward primers, nested to various parts of DEC-205 ectodomain, yielded major PCR products of the sizes predicted in accordance with the primer combinations used. We also detected slightly smaller (by 168 bp) minor PCR products, which were most apparent in the primer combinations of 078/085 and 088/085. When the DCL-1-specific reverse primer 086 was used in combination with the same DEC-205-specific forward primers, we detected doublet bands (˜200 bp apart). The larger band of which was the predicted size. Sequence analysis indicated that the smaller RT-PCR fragments from DEC-205 itself or the DEC-205/DCL-1 fusion mRNA were amplified from alternatively spliced RNA, lacking DEC-205 exon 34 (described below). Thus, the DEC-205/DCL-1 fusion mRNA encodes the entire DEC-205 ectodomain, but may also lack DEC-205 exon 34 in an alternatively spliced variant.

The DEC-205/DCL-1 Fusion mRNA is Predominantly Expressed by HRS Cell Lines

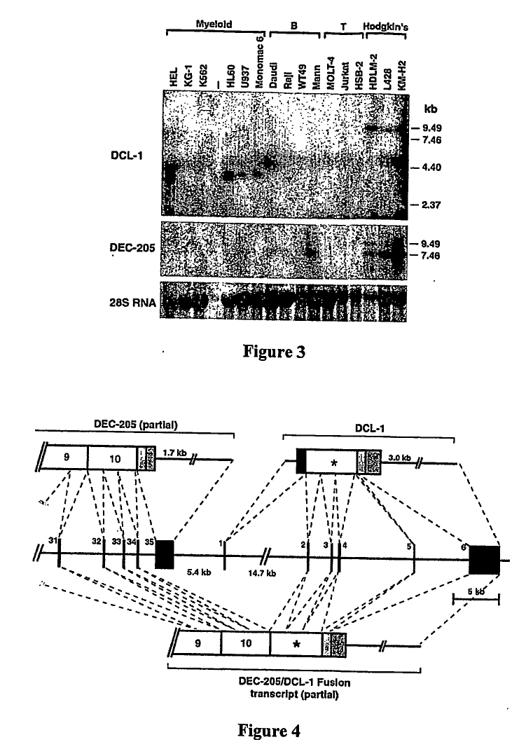

To assess DEC-205/DCL-1 fusion mRNA expression, we performed Northern blot analysis in several hematopoietic cell lines (FIG. 3). The DCL-1-specific probe nested within the DCL-1 ectodomain detected a single 4.2 kb DCL-1 mRNA band in myeloid cell lines (HEL, HL60, U937 and Monomac 6), but there were no band detected in the B or T cell lines tested. We detected a single 9.5 kb DEC-205/DCL-1 mRNA band in HRS cell lines (HDLM-2, L428 and KM-H2), however, we did not detect the 4.2 kb DCL-1 mRNA band observed in the myeloid cell lines. The U937 appear to express a small amount of the 9.5 kb DEC-205/DCL-1 mRNA in addition to the 4.2 kb DCL-1 mRNA band. When DEC-205-specific probe nested within the CR was used to hybridize the same blot after the DCL-1 probe was stripped, a 7.5 kb DEC-205 mRNA band was detected in myeloid cell lines (HEL and U937), B cell lines (Daudi and Mann) and all HRS cell lines. In addition, we detected a 9.5 kb DEC-205/DCL-1 mRNA band in all HRS cell lines and the U937 as described previously.¹⁷ Thus, it appears that DEC-205/DCL-1 fusion mRNA is predominated in HRS cell lines.

The DEC-205 and DCL-1 Gene are Juxtaposed in Chromosome Band 2Q24

We mapped the DEC-205 gene previously to the chromosome band 2q24.¹⁷ The KIAA0022/DCL-1 gene was previously located to chromosome 222 and further mapped recently to the identical chromosomal band in the NCBI UniGene database. Using the NCBI Genome BLAST, we identified the human genomic contig NT 005151 containing both DEC-205 and the DCL-1 gene. Our sequence analysis showed that DEC-205 and DCL-1 genes consist of 35 and 6 exons, respectively, and the DEC-205 gene is localized ˜5.4 kb upstream of the DCL-1 gene (FIG. 4). Therefore, the DEC-205 and DCL-1 fusion mRNA appears to be generated by cotranscription of both DEC-205 and DCL-1 genes followed by intergenic splicing to remove the DEC-205 exon 35 (or exon 34/35) and DCL-1 exon 1.

DEC-205/DCL-1 Fusion mRNA is Translated to the Fusion Protein

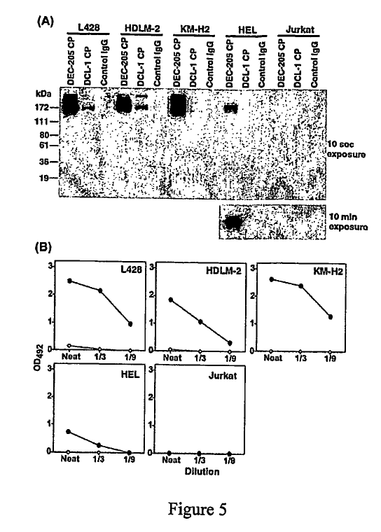

We sought to establish whether the DEC-205/DCL-1 fusion mRNA is translated into a fusion protein. We prepared cell lysates from three HRS cell lines (DEC-205 mRNA⁺, DEC-205/DCL-1 fusion mRNA⁺), HEL (DEC-205 mRNA⁺, DEC-205/DCL-1 fusion mRNA⁻) and Jurkat cell line (DEC-205 mRNA⁻, DEC-205/DCL-1 fusion mRNA⁻)(see FIG. 3), and subjected them to immunoprecipitation with the DEC-205 CP or DCL-1 CP peptide antisera. The immunoprecipitated samples were further analyzed by Western blot with DEC-205 mAbs to detect DEC-205 and DEC-205/DCL-1 fusion protein in non-reducing conditions (FIG. 5A). The DEC-205 CP antiserum precipitated a broad but single ˜180 kDa DEC-205 protein band specifically from the three HRS cell lines (L428, HDLM-2 and KM-H2) and HEL cells. There was no detectable signal in Jurkat cells. When the DCL-1 CP antiserum was used for the initial immunoprecipitation, we detected low levels of ˜180 kDa DEC-205/DCL-1 fusion protein band in the three HRS cell lines, but not in HEL or Jurkat cells. The presence of this DEC-205/DCL-1 fusion protein band in these HRS cell extracts was not due to cross-reactivity of DCL-1 CP antiserum with DEC-205 CP because (i) there was no cross-reactivity in the DCL-1 CP antiserum with DEC-205 CP peptide assessed by ELISA analysis (data not shown), (ii) 60 times longer exposure of HEL sample did not produce any band (FIG. 5A) and (iii) the DCL-1 CP antiserum detected the weakest signal in KM-H2 extracts, which contained most DEC-205 protein (FIG. 5A and described below).

To determine the relative abundance of the DEC-205/DCL-1 fusion protein to DEC-205, we developed a sandwich ELISA using the DEC-205 mAbs for capturing and the CP antisera for detection (FIG. 5B). The HRS cell lines express most DEC-205 protein (KM-H2>L428>HDLM-2), followed by HEL cells. We detected relatively small amounts of the DEC-205/DCL-1 fusion protein in L428 and HDLM-2 cells, approximately 30-50 times less than the amount of DEC-205. No fusion protein was detected in the KM-H2 cells, probably because the amount of KM-H2 derived fusion protein is below the detection limit. The negative control, Jurkat, did not show any signal. The relative abundance of both DEC-205 and DEC-205/DCL-1 fusion protein by the ELISA correlated with the immunoprecipitation/Western blot data (FIG. 5A).

### EXAMPLE 2

### Identification and Analysis of DCL-1

Identification of DCL-1 cDNA

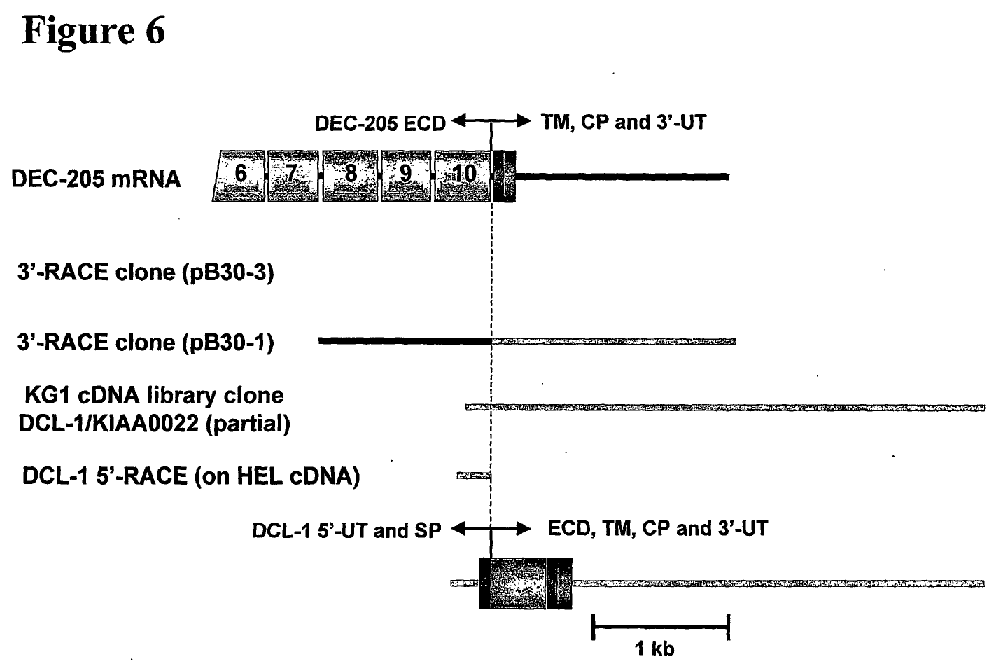

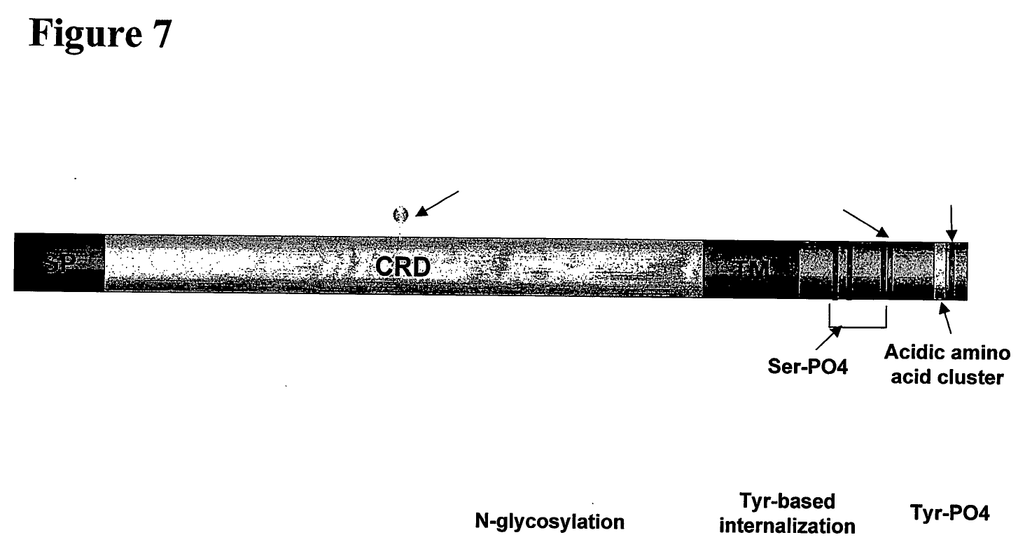

DCL-1 cDNA was identified as a genetic fusion partner of DEC-205 in Hodgkin's disease-derived cell line L428 by 3′-rapid amplification of cDNA ends (RACE). GenBank search identified a partial DCL-1 cDNA clone KIAA0022. 5′-RACE was performed and amplified ˜250 bp fragment to complete DCL-1 cDNA (details published in Masato et al., J. Biol. Chem. 2003) and annotated its protein structure (FIG. 6).

Analysis of the DCL-1 protein revealed that it is a putative type 1 transmembrane C-type lectin receptor of 232 amino acids. Its extracellular domain contains only one carbohydrate recognition domain and one end glycosylation site within the carbohydrate recognition domain. Its cytoplasmic portion contains several motives for SER-PO₄, tyrosine based internalisation, late endosome targetting and Tyr-PO₄. DCL-1 is highly conserved between species. For example, it is approximately 80% conserved between human and mouse.

Northern Blot Analysis of Hematopoietic Cell Lines for DCL-1 mRNA Expression

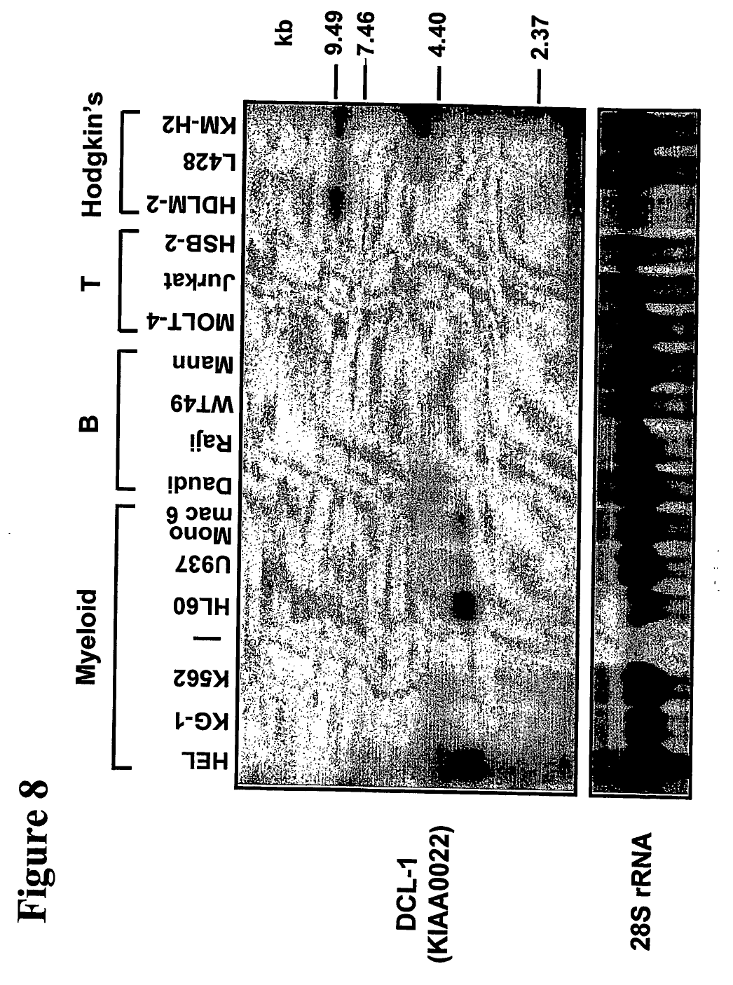

Total RNA from hematopoietic cell lines was purified using Trizol, fractionated with denaturing formaldehyde gel electrophoresis. The RNA was transferred onto a cationic nylon membrane and probed with [32P]-labeled DCL-1 specific cDNA probe (details published in Masato et al., J. Biol. Chem., 2003). The 4.2 kb DCL-1 mRNA expression was restricted in myeloid cell lines, but not in B and T cell lines. In Hodgkin's disease-derived cell lines, only 9.5 kb mRNA corresponding to DEC-205/DCL-1 fusion transcript was detected (FIG. 8).

DCL-1 Gene Structure

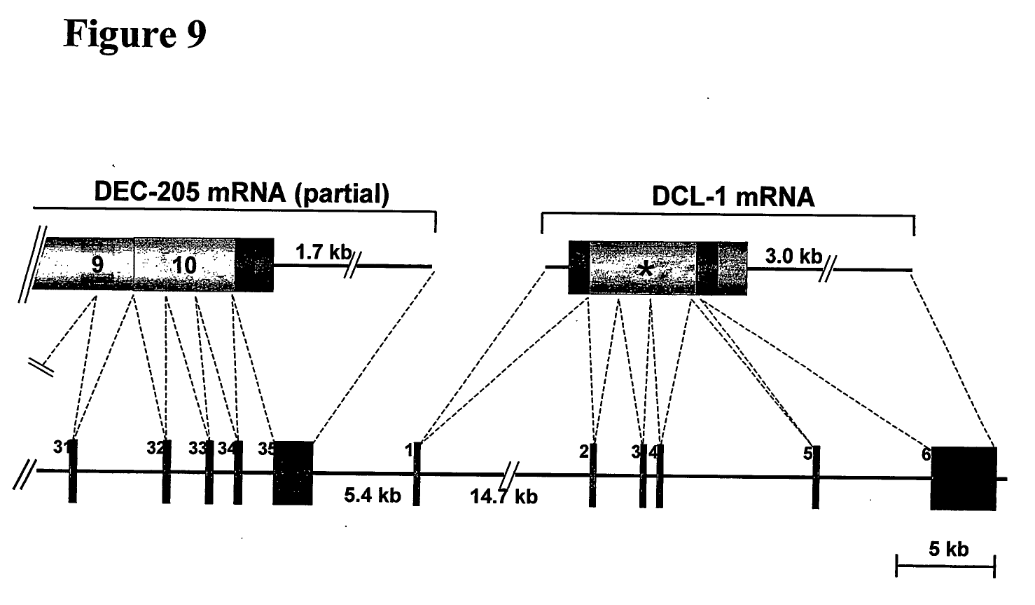

The DCL-1 gene consists of 6 exons; exon 1 encodes 5′-untranslated region (UT) and a signal peptide, exon 2-4 encode a carbohydrate recognition domain (CRD), exon 5 encodes a stalk region connecting DCL-1 extracellular domain to a transmembrane domain, and exon 6 encodes cytoplasmic domain (CP) and 3′-UT (FIG. 9). The DCL-1 gene is mapped onto chromosome band 2q24 and 5.4 kb downstream of DEC-205 gene. Exon 5 may be alternatively spliced according to the mouse DCL-1 cDNA analysis.

DCL-1 Protein Expression in FLAG-tagged DCL-1 Transfectants

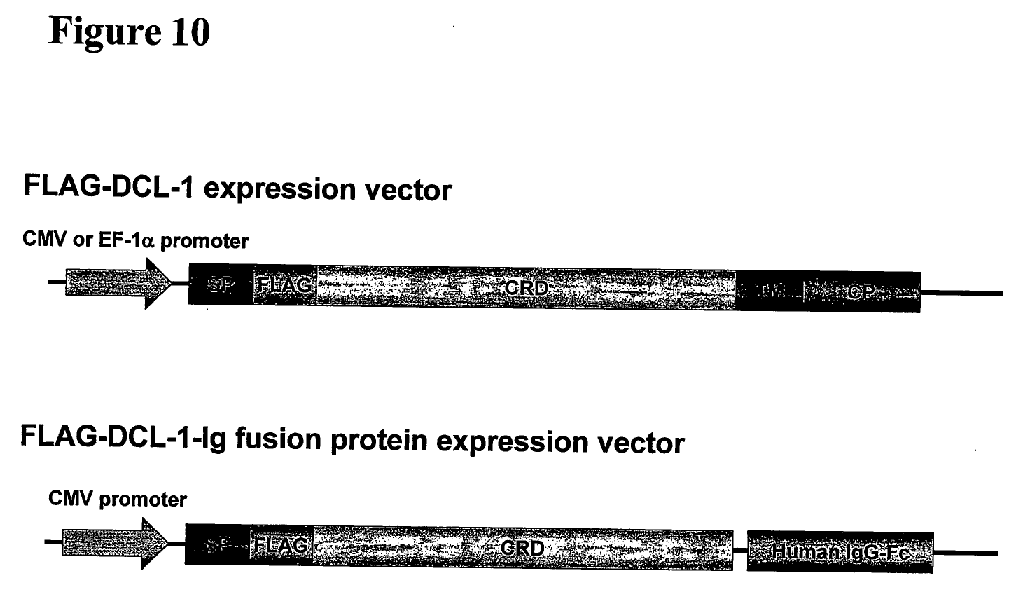

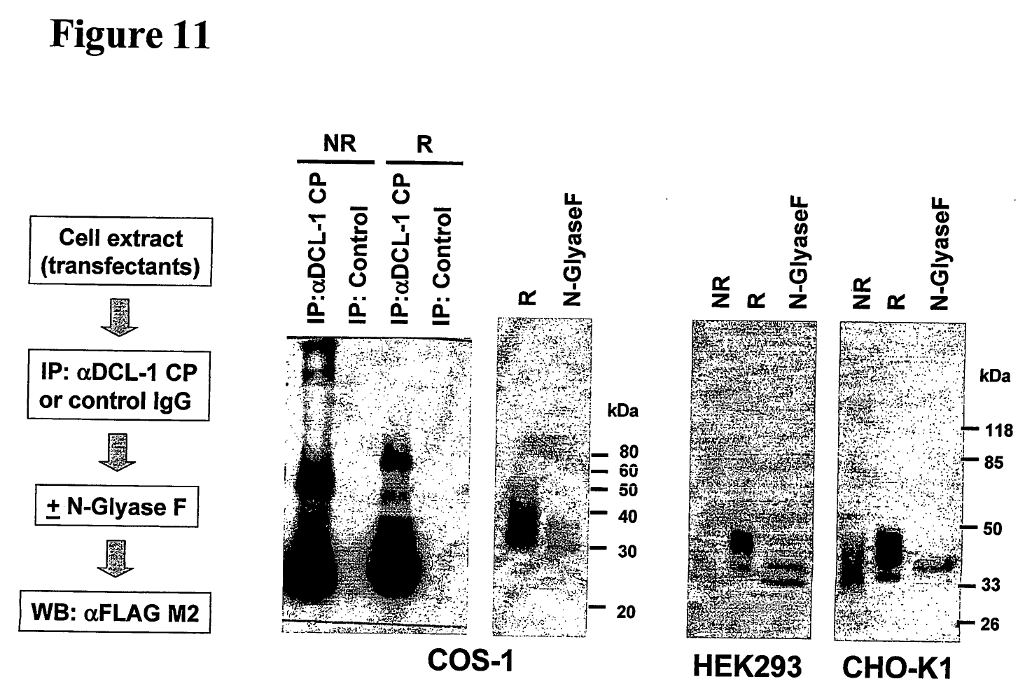

A FLAG-tagged DCL-1 mammalian expression vector (FIG. 10) was constructed and transfected into COS-7 (transient transfection), HEK293 (stable transfection) and CHO-K1 cells (stable transfection). The transfectants were extracted with a immunoprecipitation buffer (RIPA buffer) and immunoprecipitated with rabbit anti DCL-1 CP and protein A agarose. The precipitated protein was treated with or without N-glycosidase F, fractionated with SDS-PAGE in reducing or non-reducing condition, and transferred onto a PVDF membrane. FLAG-DCL-1 protein appeared to be 30-40 kDa protein in a reducing condition. N-glycosidase F treatment reduce FLAG-DCL-1 molecular mass indicating that DCL-1 is N-N-glycosylated at an N-glycosylation site in the CRD (FIG. 11).

Expression of DCL-1 mRNA and Protein in Purified Leukocytes

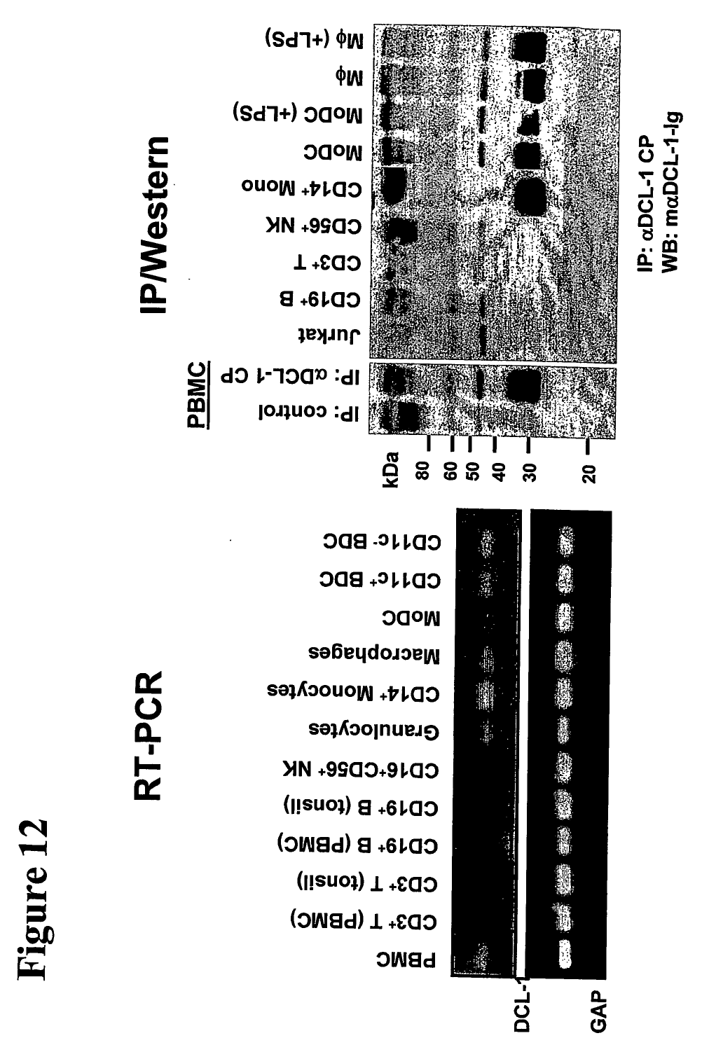

Total RNA was purified from flow purified leukocytes using Trizol and subjected to RT-PCR for expression of DCL-1 mRNA and GAPDH (house keeping gene, a control for cDNA input normalization). DCL-1 mRNA expression was only detected in phagocytic cells (i.e. granulocytes, monocytes, macrophages) and dendritic cells (i.e. monocyte-derived dendritic cells, blood CD11c+ dendritic cells and CD11c− dendritic cells), but not in T, B, NK cells (FIG. 12, left panel).

Purified leukocytes were extracted with a immunoprecipitation buffer (RIPA buffer) and immunoprecipitated with rabbit anti DCL-1 CP and protein A agarose. The precipitated protein was fractionated with SDS-PAGE non-reducing condition, and transferred onto a PVDF membrane. The membrane was probed with mouse antiserum made against the FLAG-tagged DCL-1-Ig fusion protein. The DCL-1 protein appears to be expressed in phagocytes (i.e. monocytes, macrophages) and dendritic cells (i.e. monocyte-derived dendritic cells), but not in T, B and NK cells. Jurkat cells (T cell line) was used as a negative control (FIG. 12, right panel). The result is consistent with the RT-PCR results.

Strategy for Production of Monoclonal Antibodies Against Human DCL-1

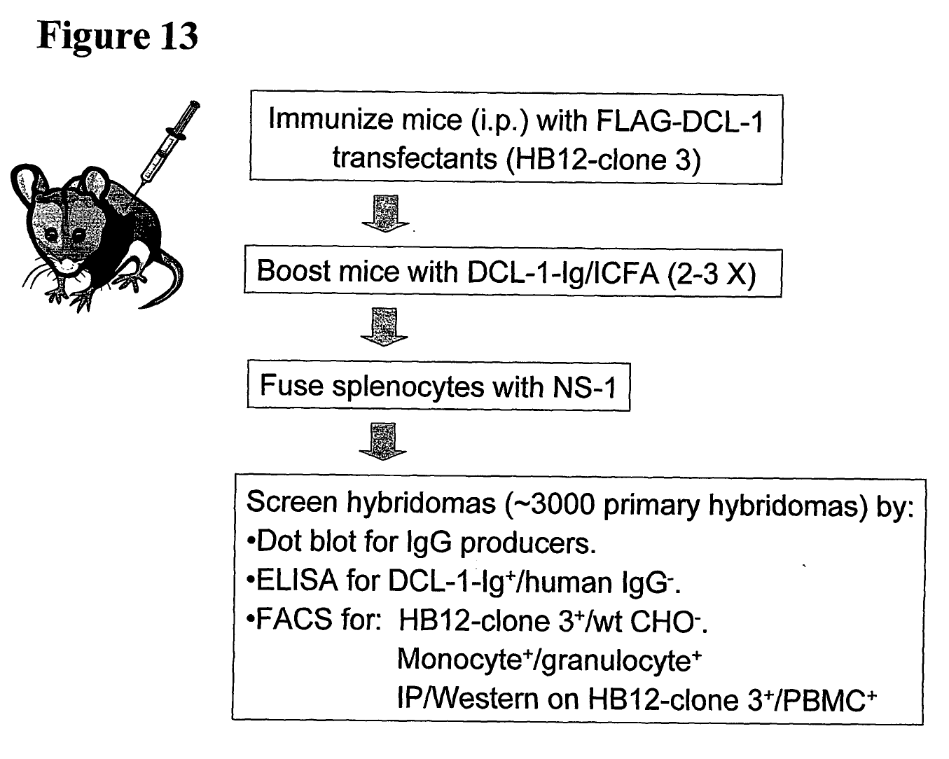

Mice were immunized with CHO-K1 stable transfectants expressing the FLAG-DCL-1 (HB12-clone 3) and boosted 2-3 times with the FLAG-DCL-1-Ig fusion protein (FIG. 13). The mice spleens were harvested and fused with NS-1 for hybridoma production. Approximately 3000 hybridomas were screened for mouse IgG producers by dot blot analysis, FLAG-DCL-1+ but human IgG− hybridomas by ELISA and HB12-clone 3+/wild type CHO-K1−, monocyte+/granulocyte+ by flow cytometry and HB 12-clone 3+/PBMC+ by immunoprecipitation/western blot analysis. 5 hybridomas were derived from independent primary hybridomas.

Flow Cytometry Analysis of DCL-1 Expression on Peripheral Blood Mononuclear Cells Using Monoclonal Antibodies Against DCL-1

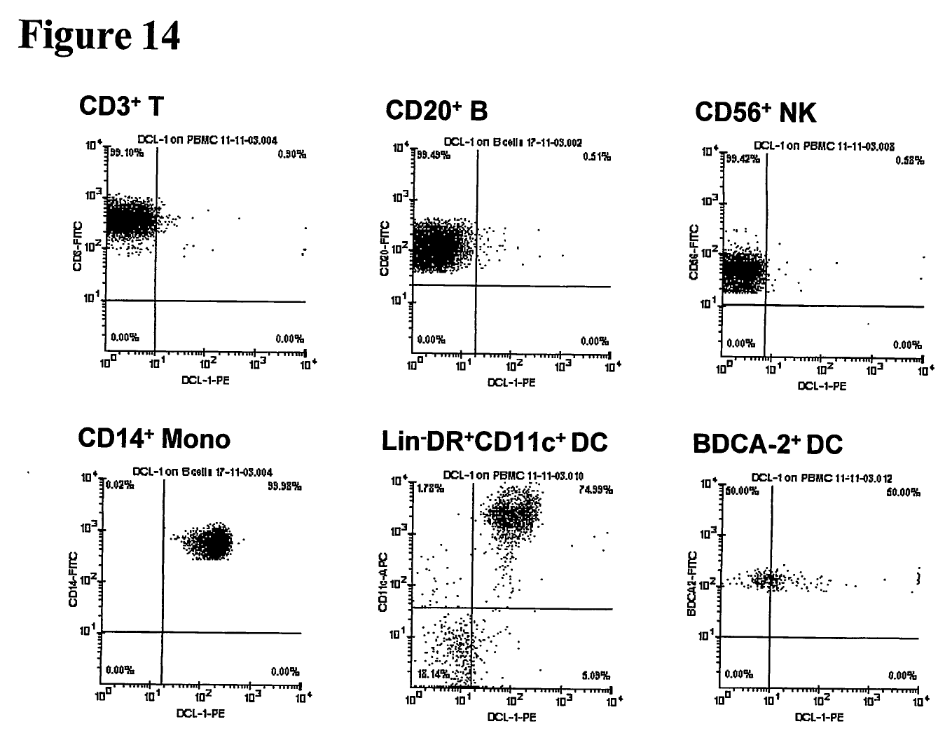

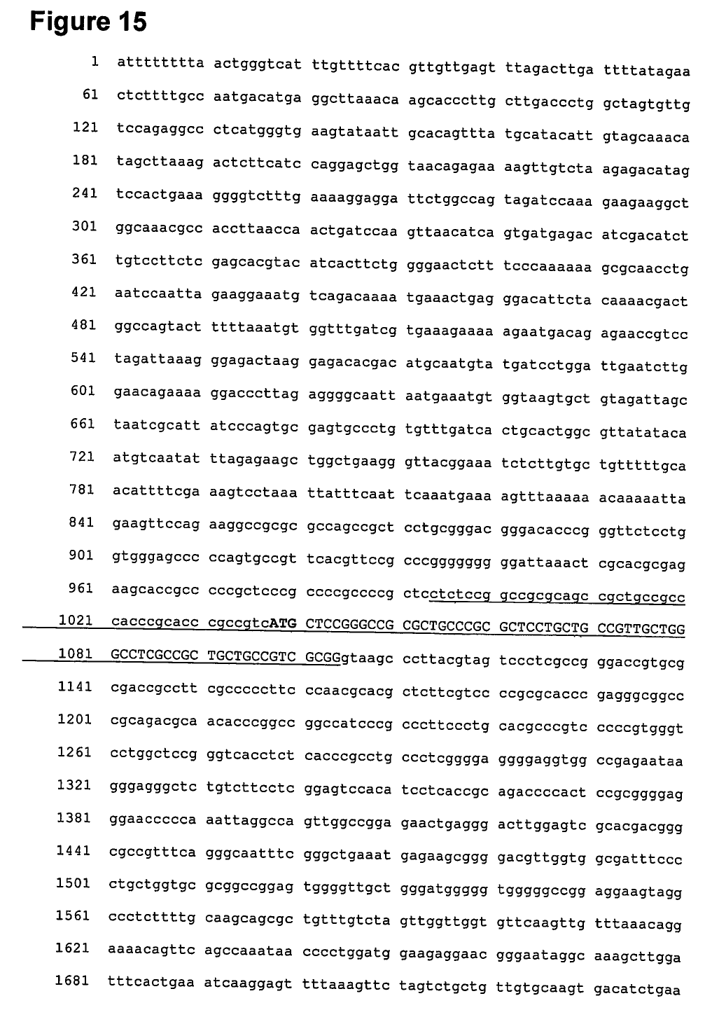

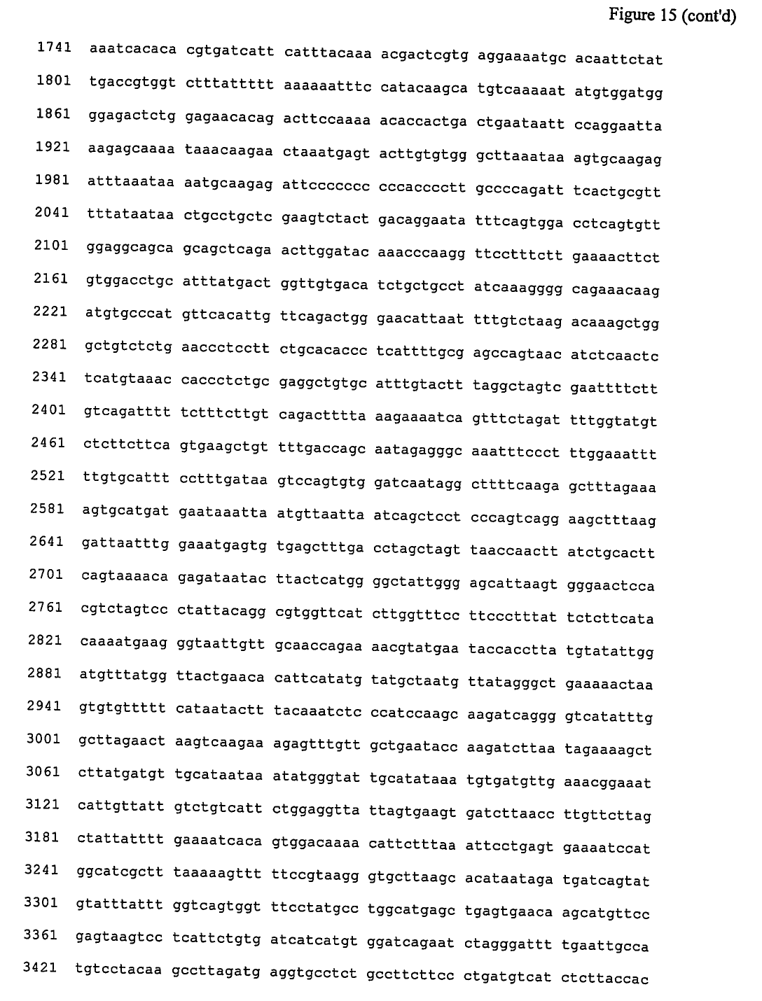

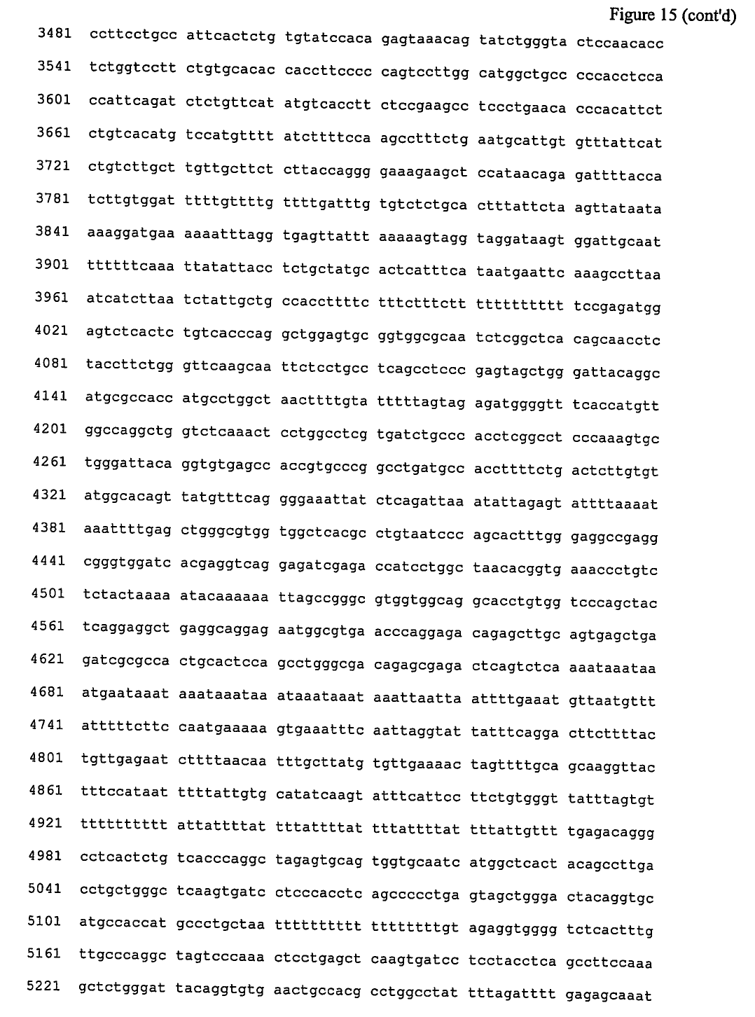

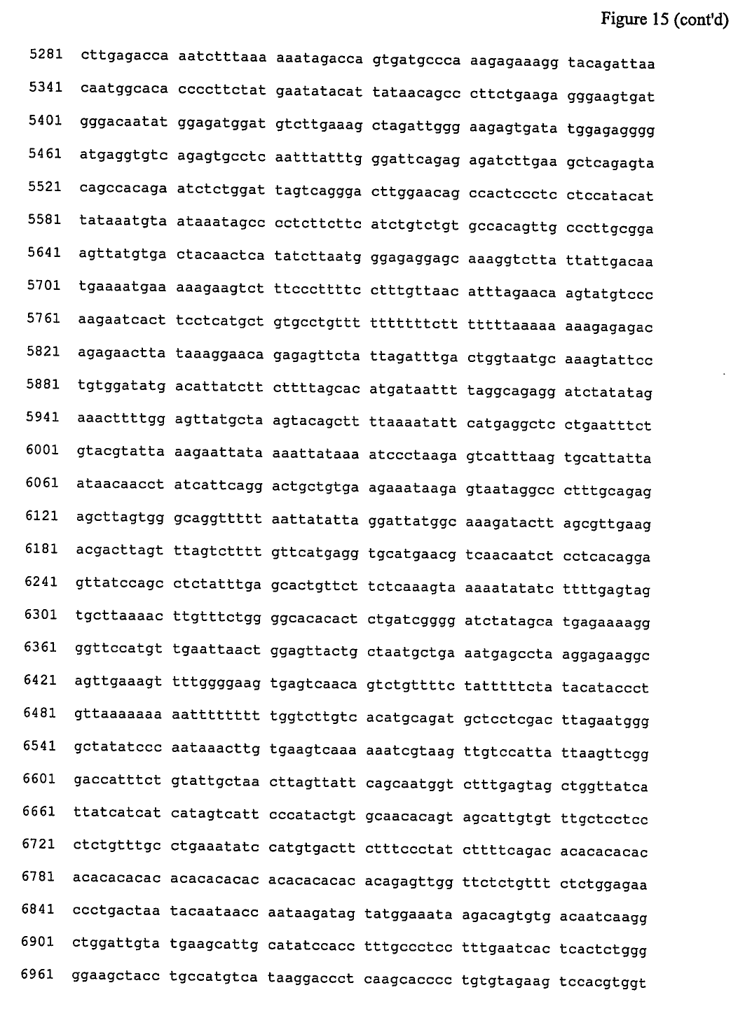

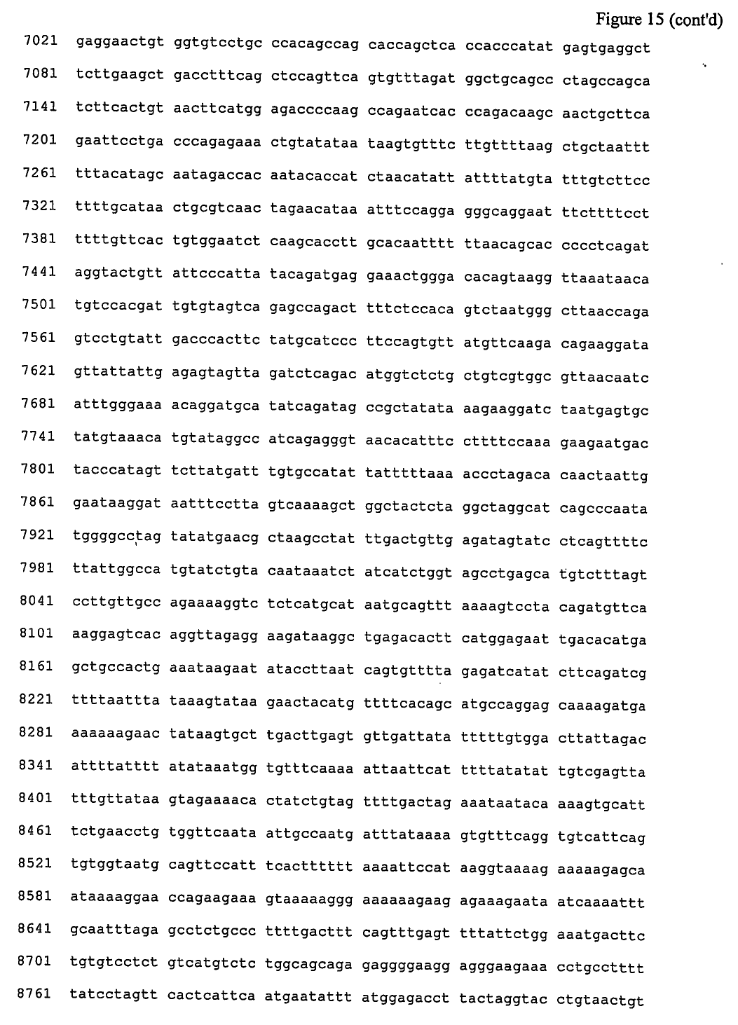

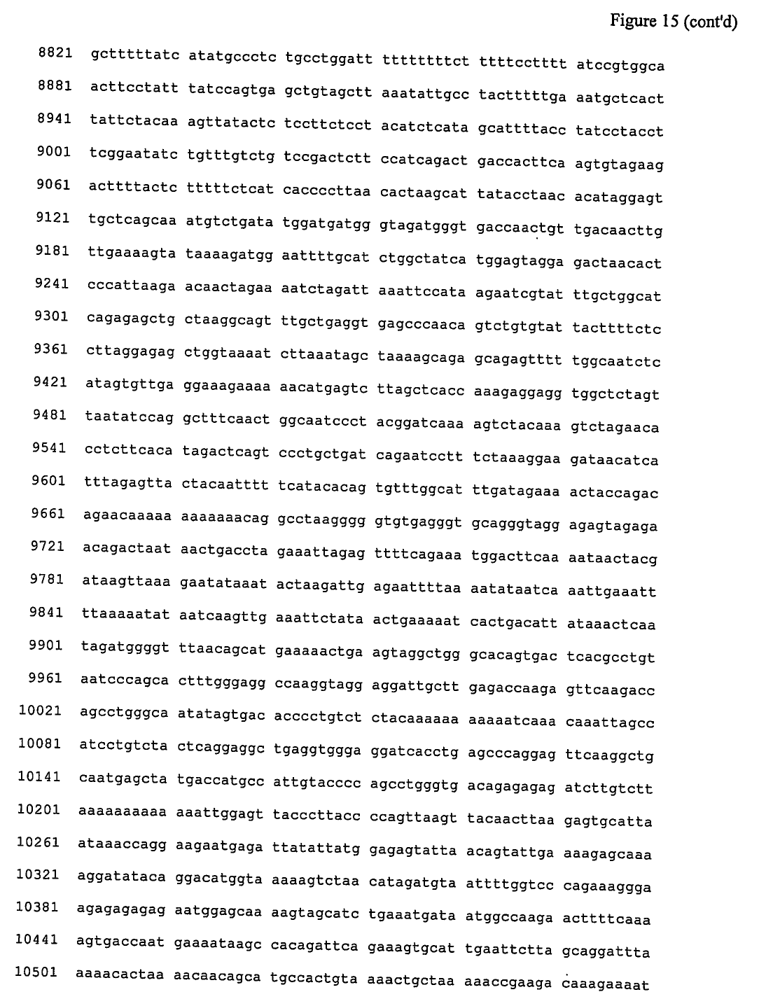

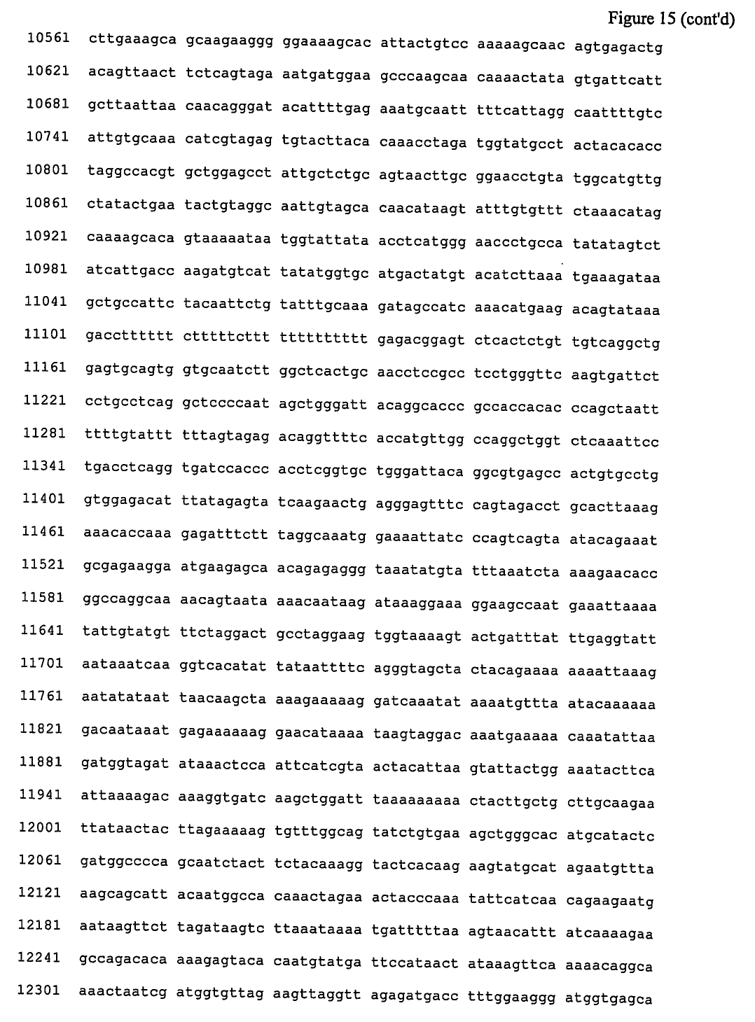

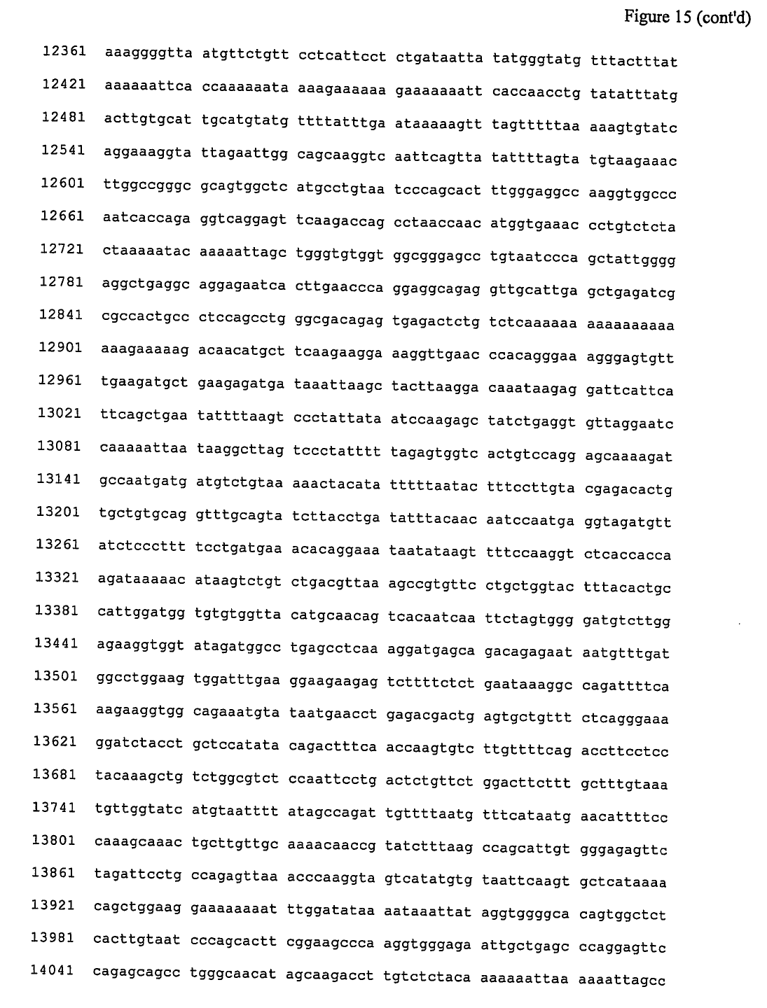

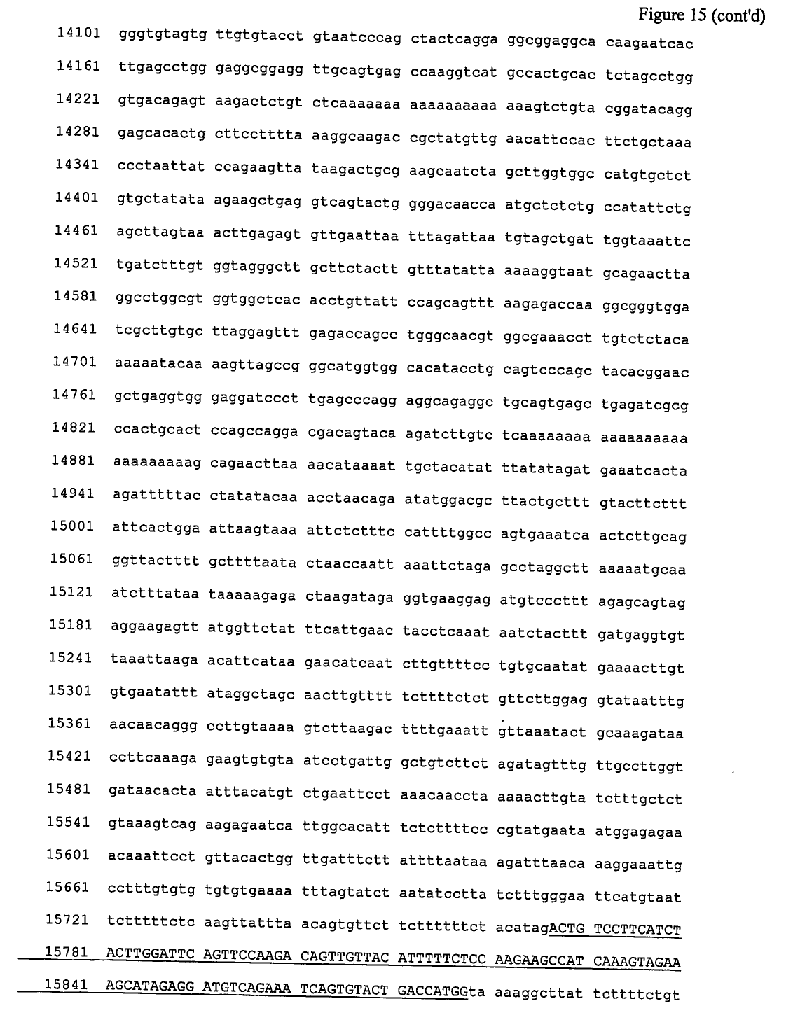

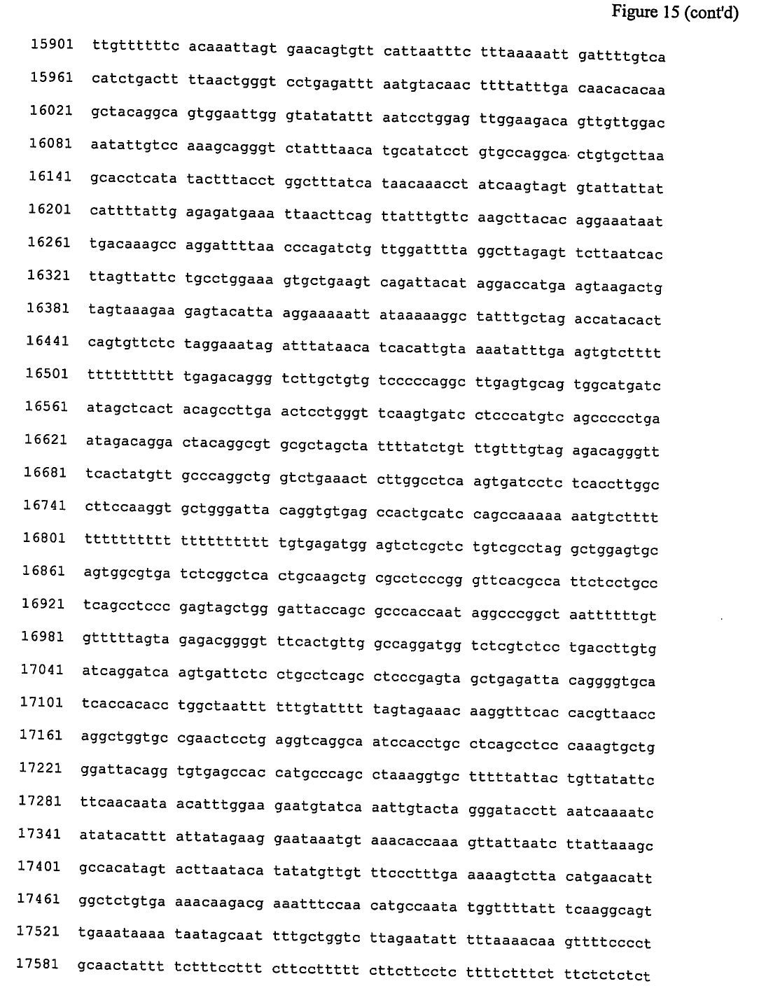

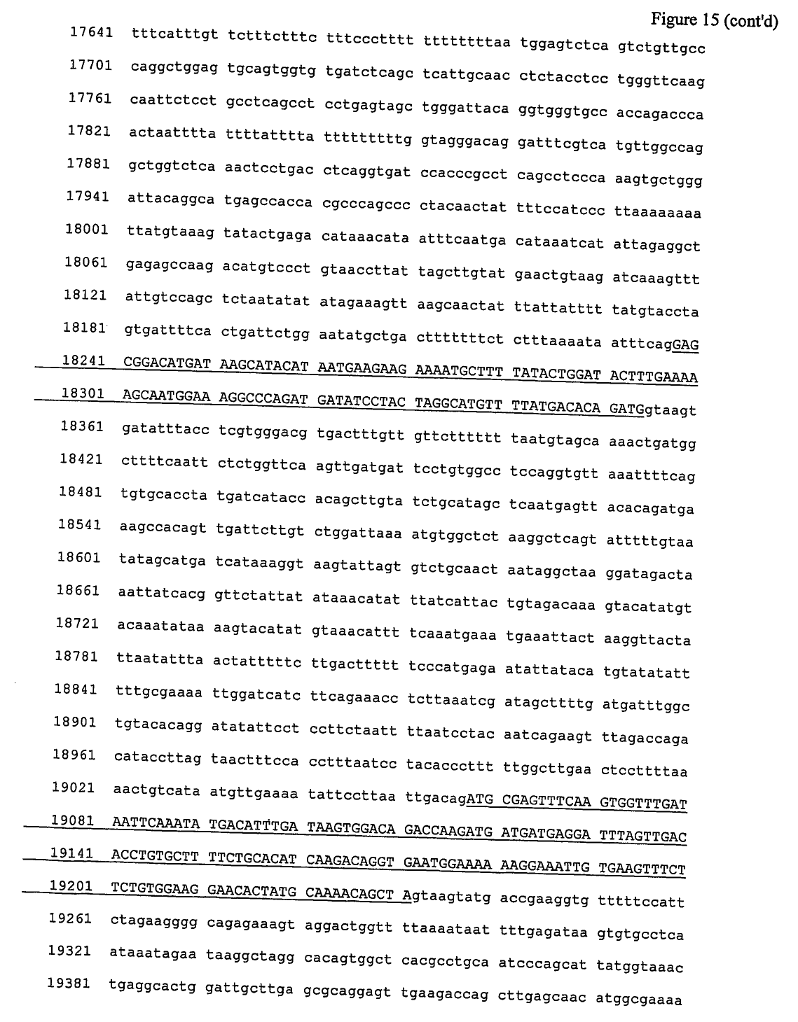

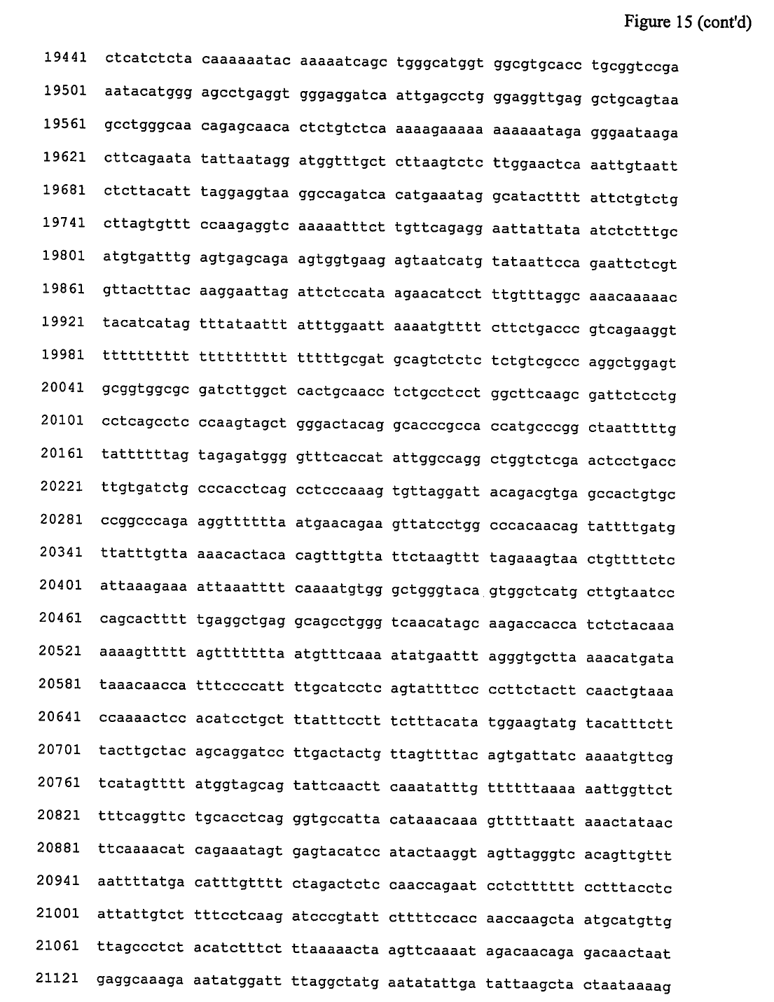

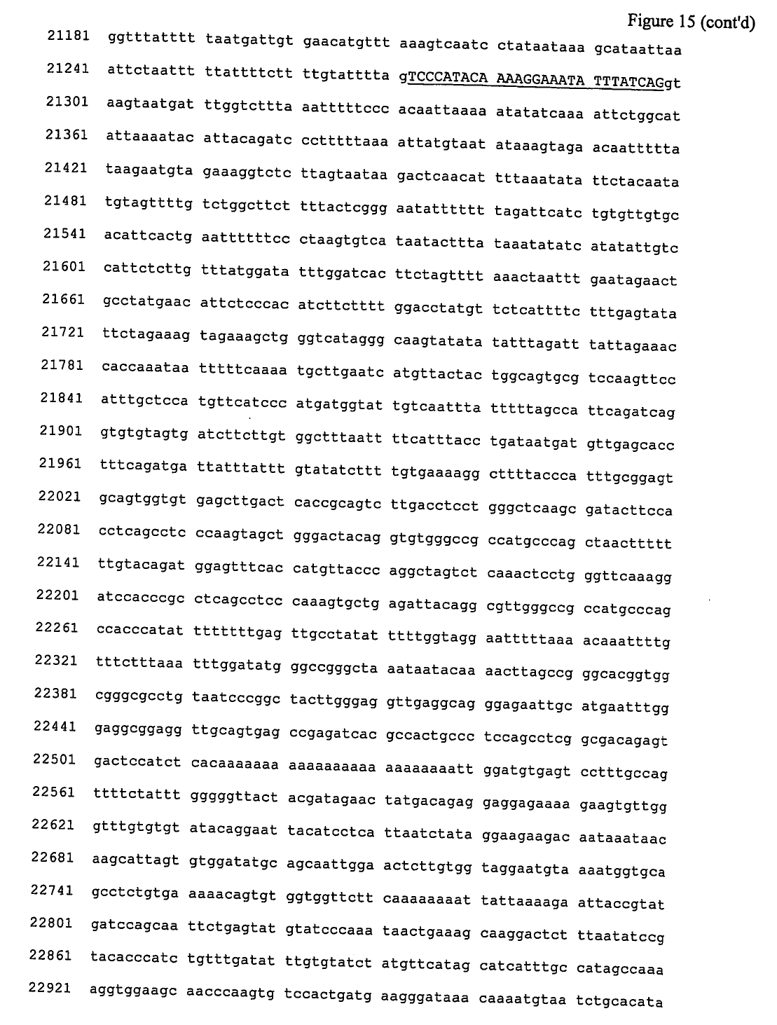

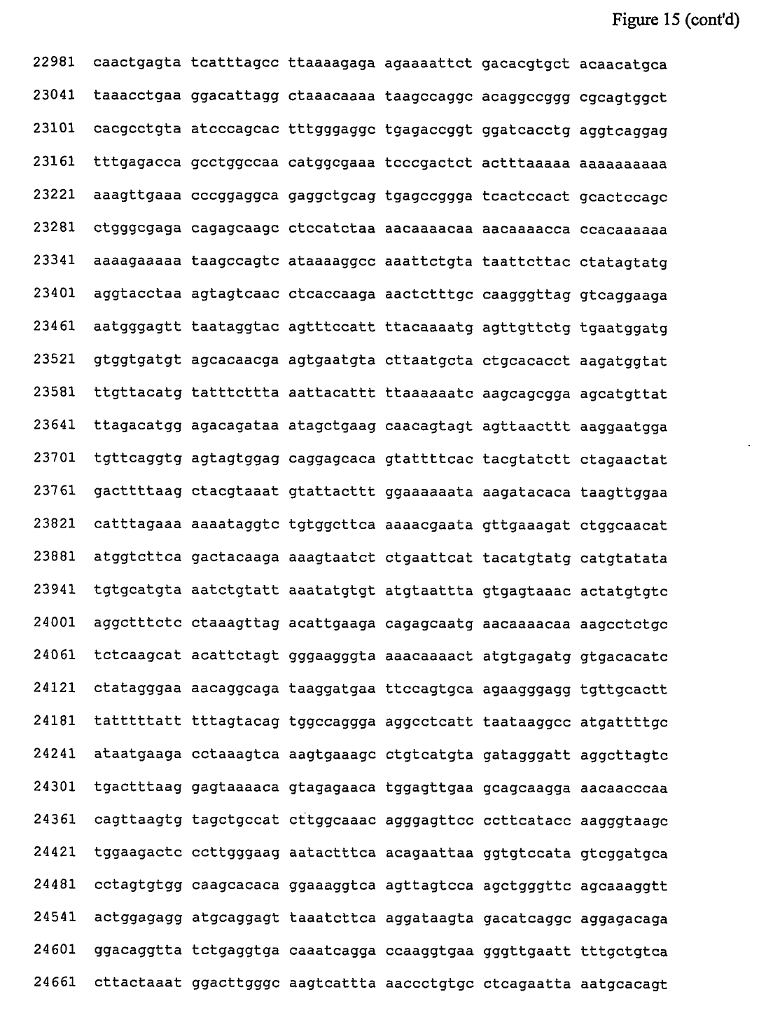

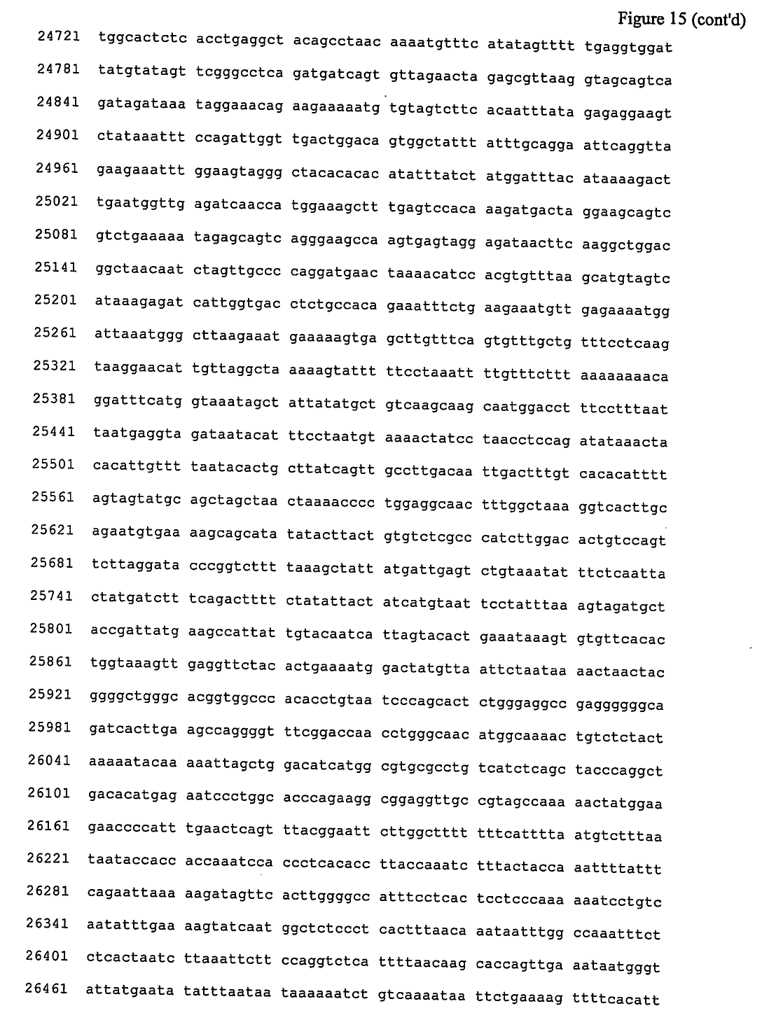

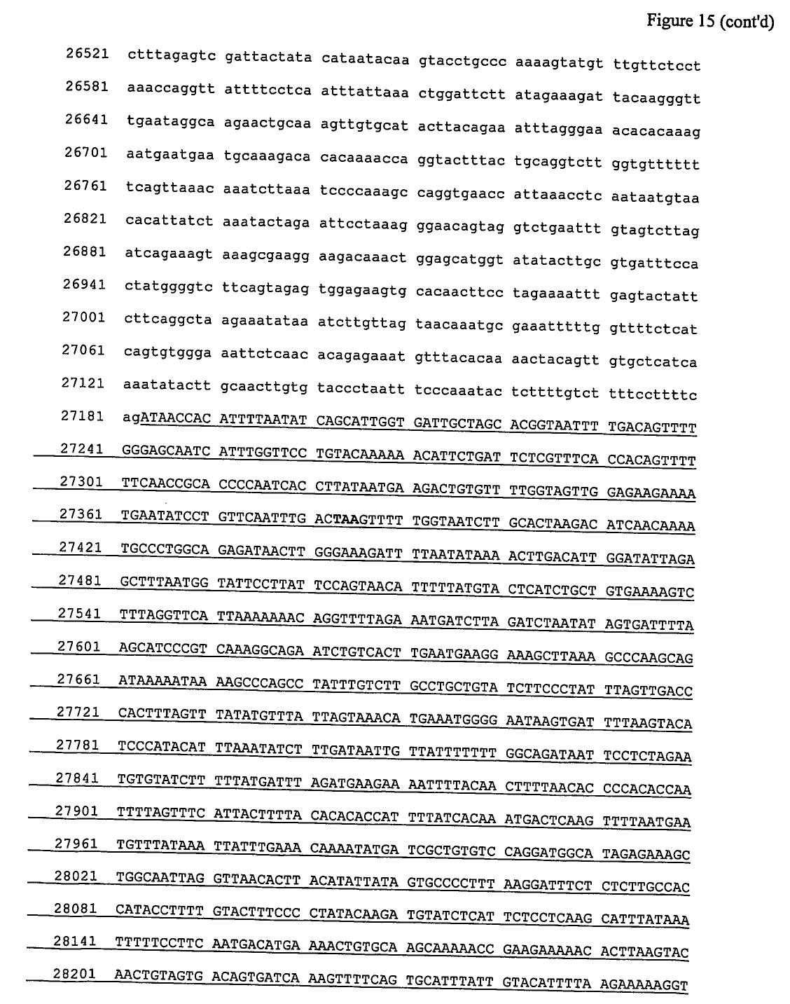

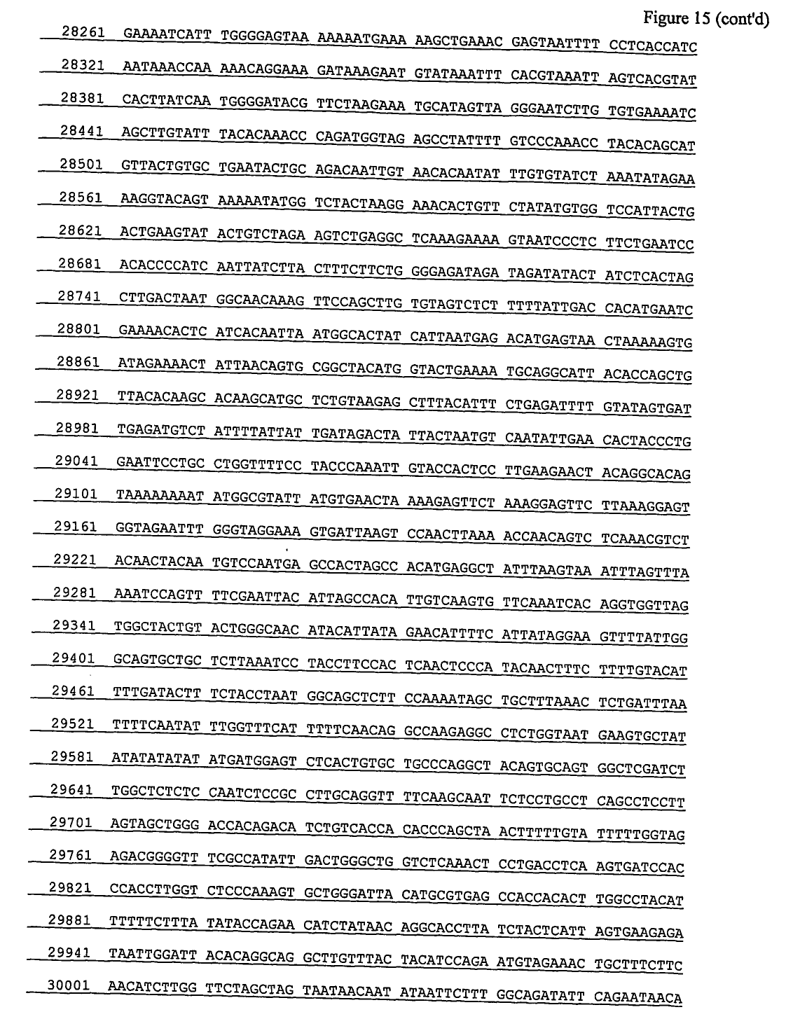

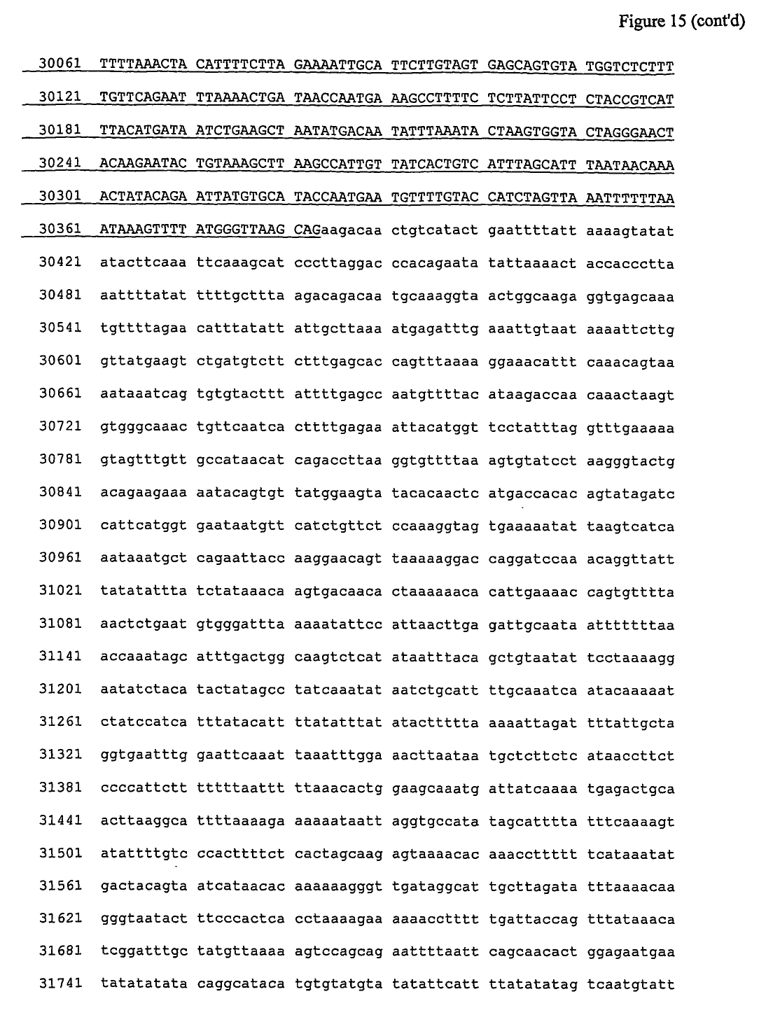

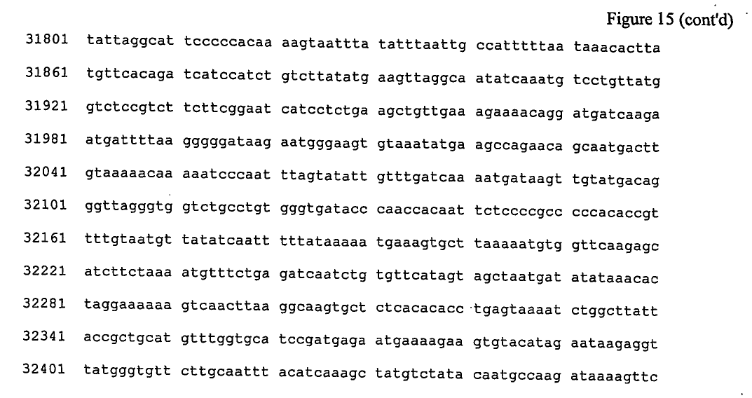

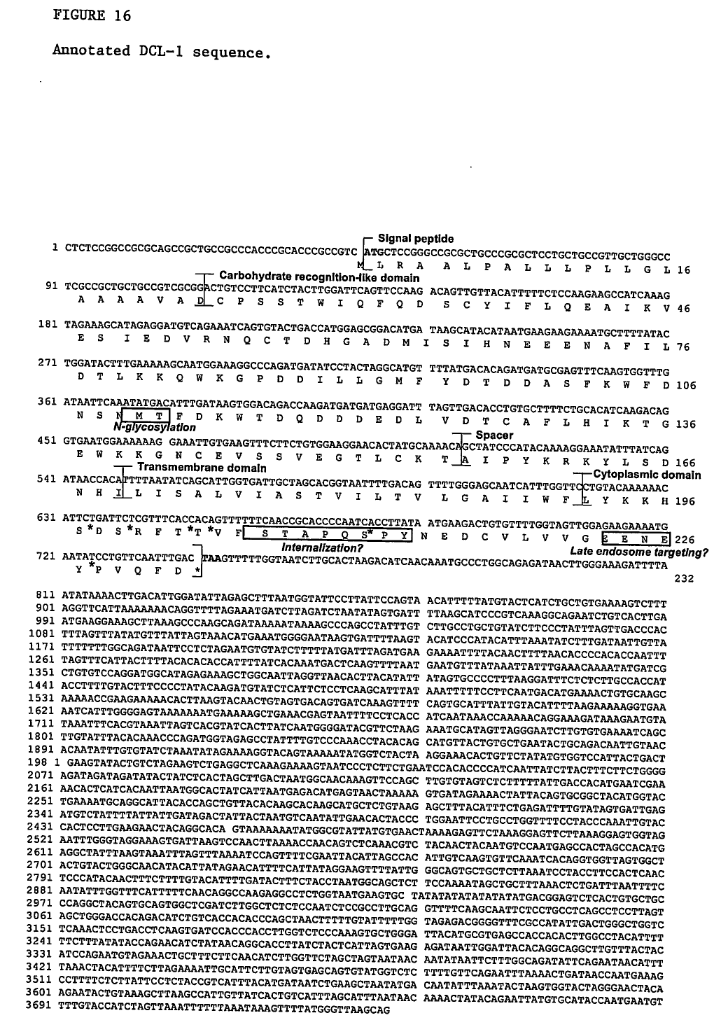

Peripheral blood mononuclear cells (PBMC) were double stained with a DCL-1 mAb and lineage antibodies for DCL-1 cell surface expression. DCL-1 was expressed on CD14+ monocytes, CD11c+ blood dendritic cells (myeloid subset) and BDCA-2+ blood DC (plasmacytoid subset, CD11c−) (FIG. 14).

Summary

DCL-1 is a 30 kDa novel C-type lectin receptor encoded by a 4.2 kb mRNA. The DCL-1 gene consists of 6 exons and is localized downstream of the DEC-205 gene. Monoclonal antibody against DCL-1 has now been produced. DCL-1 is expressed only on phagocytes (i.e. monocytes, macrophages, granulocytes) and dendritic cells, but not on B, T or NK cells.

DCL-1 is involved in endocytic and signalling function of phagocytes and dendritic cells and may be used as an antigen loading receptor to dendritic cells for dendritic cell immunotherapy.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Ancian P, Lambeau G, Mattei M G, Lazdunski M: The human 180-kDa receptor for secretory phospholipases A2. Molecular cloning, identification of a secreted soluble form, expression, and chromosomal localization. J. Biol. Chem. 1995;270:8963-8970.
Banchereau J, Steinman R M: Dendritic cells and the control of immunity. Nature. 1998;392:245-252.
Communi D, Suarez-Huerta N, Dussossoy D, Savi P, Boeynaems J M: Cotranscription and intergenic splicing of human P2Y11 and SSF1 genes. J. Biol. Chem. 2001;276:16561-16566.
Dekker J W, Budhia S, Clark G J, Hart D N J, Kato M: Identification of a S-Adenosylhomocysteine Hydrolase-Like Transcript Induced During Dendritic Cell Differentiation. Immunogenetics. 2002;53:993-1001
Delabie J, Ceuppens J L, Vandenberghe P, et al.: The B7/BB1 antigen is expressed by Reed-Sternberg cells of Hodgkin's disease and contributes to the stimulating capacity of Hodgkin's disease-derived cell lines. Blood. 1993;82:2845-2852.
Diehl V, Pfreundschuh M, Fonatsch C, et al.: Phenotypic and genotypic analysis of Hodgkin's disease derived cell lines: histopathological and clinical implications. Cancer Surv. 1985;4:399-419
Ellis P A, Hart D N, Colls B M, et al.: Hodgkin's cells express a novel pattern of adhesion molecules. Clin. Exp. Immunol. 1992;90:117-123.
Ezekowitz R A, Sastry K, Bailly P, Warner A: Molecular characterization of the human macrophage mannose receptor: demonstration of multiple carbohydrate recognition-like domains andphagocytosis of yeasts in Cos-1 cells. J. Exp. Med. 1990;172:1785-1794.
Falini B, Pileri S, Pizzolo G, et al.: CD30 (Ki-1) molecule: a new cytokine receptor of the tumor necrosis factor receptor superfamily as a tool for diagnosis and immunotherapy. Blood. 1995;85:1-14.
Fears S, Mathieu C, Zeleznik-Le N, et al.: Intergenic splicing of MDS1 and EVI1 occurs in normal tissues as well as in myeloid leukemia and produces a new member of the PR domain family. Proc. Natl. Acad. Sci. USA. 1996;93:1642-1647.
Gruss H J, Hirschstein D, Wright B, et al.: Expression and function of CD40 on Hodgkin and Reed-Sternberg cells and the possible relevance for Hodgkin's disease. Blood. 1994;84:2305-2314.
Hart D N: Dendritic cells: unique leukocyte populations which control the primary immune response. Blood. 1997;90:3245-3287.
Hock B D, Fearnley D B, Boyce A, et al.: Human dendritic cells express a 95 kDa activation/differentiation antigen defined by CMRF-56. Tissue Antigens. 1999;53:320-334.
Hock B D, Kato M, McKenzie J L, Hart D N: A soluble form of CD83 is released from activated dendritic cells and B lymphocytes, and is detectable in normal human sera. Int. Immunol. 2001;13:959-967.
Hock B D, Starling G C, Daniel P B, Hart D N: Characterization of CMRF-44, a novel monoclonal antibody to an activation antigen expressed by the allostimulatory cells within peripheral blood, including dendritic cells. Immunology. 1994;83:573-581.
Howard M J, Isacke C M: The C-type Lectin Receptor Endo 180 Displays Internalization and Recycling Properties Distinct from Other Members of the Mannose Receptor Family. J. Biol. Chem. 2002;277:32320-32331.
Ishizaki J, Hanasaki K, Higashino K, et al.: Molecular cloning of pancreatic group I phospholipase A2 receptor. J. Biol. Chem. 1994;269:5897-5904.
Jiang W, Swiggard W J, Heufler C, et al.: The receptor DEC-205 expressed by dendritic cells and thymic epithelial cells is involved in antigen processing. Nature. 1995;375:151-155.
Kamesaki H, Fukuhara S, Tatsumi E, et al.: Cytochemical, immunologic, chromosomal, and molecular genetic analysis of a novel cell line derived from Hodgkin's disease. Blood. 1986;68:285-292
Kapp U, Yeh W C, Patterson B, et al.: Interleukin 13 is secreted by and stimulates the growth of Hodgkin and Reed-Sternberg cells. J. Exp. Med. 1999;189:1939-1946.
Kato M, MacDonald K, Munster D, Clark G, Hart D N J: DEC 205 workshop panel report, in Mason D (ed): Leucocyte Typing VII. Oxford, Oxford University Press, 2002, p 298-330
Kato M, Neil T K, Clark G J, et al.: cDNA cloning of human DEC-205, a putative antigen-uptake receptor on dendritic cells. Immunogenetics. 1998;47:442-450.
Keilholz U, Szelenyi H, Siehl J, et al.: Rapid regression of chemotherapy refractory lymphocyte predominant Hodgkin's disease after administration of rituximab (anti CD 20 mono-clonal antibody) and interleukin-2. Leuk. Lymphoma. 1999;35:641-642.
Kretschmer C, Jones D B, Morrison K, et al.: Tumor necrosis factor alpha and lymphotoxin production in Hodgkin's disease. Am. J. Pathol. 1990;137:341-351.
Kuppers R, Rajewsky K: The origin of Hodgkin and Reed/Sternberg cells in Hodgkin's disease. Annu. Rev. Immunol. 1998;16:471-493.
Laemmli U K: Cleavage of structural proteins during the assembly of the head of bacteriophage T4. Nature. 1970;227:680-685.
Magrangeas F, Pitiot G, Dubois S, et al.: Cotranscription and intergenic splicing of human galactose-1-phosphate uridylyltransferase and interleukin-11 receptor alpha-chain genes generate a fusion mRNA in normal cells. Implication for the production of multidomain proteins during evolution. J. Biol. Chem. 1998;273:16005-16010.
Mahnke K, Guo M, Lee S, et al.: The dendritic cell receptor for endocytosis, DEC-205, can recycle and enhance antigen presentation via major histocompatibility complex class II-positive lysosomal compartments. J. Cell. Biol. 2000;151:673-684.
Marafioti T, Hummel M, Foss H D, et al.: Hodgkin and reed-sternberg cells represent an expansion of a single clone originating from a germinal center 13-cell with functional immunoglobulin gene rearrangements but defective immunoglobulin transcription. Blood. 2000;95: 1443-1450.
McKenzie J L, Egner W, Calder V L, Hart D N: Hodgkin's disease cell lines: a model for interleukin-1-independent accessory cell function. Immunology. 1992;77:345-353.
Moore R C, Lee I Y, Silverman G L, et al.: Ataxia in prion protein (PrP)-deficient mice is associated with upregulation of the novel PrP-like protein doppel. J. Mol Biol. 1999;292:797-817.
Nestle F O, Banchereau J, Hart D: Dendritic cells: On the move from bench to bedside. Nat. Med. 2001;7:761-765.
Nomura N, Miyajima N, Sazuka T, et al.: Prediction of the coding sequences of unidentified human genes. I. The coding sequences of 40 new genes (KLA0001-KIAA0040) deduced by analysis of randomly sampled cDNA clones from human immature myeloid cell line KG-1. DNA Res. 1994;1:27-35
Paietta E, Racevskis J, Stanley E R, et al.: Expression of the macrophage growth factor, CSF-1 and its receptor c-fms by a Hodgkin's disease-derived cell line and its variants. Cancer Res. 1990;50:2049-2055.
Schaadt M, Diehl V, Stein H, Fonatsch C, Kirchner H H: Two neoplastic cell lines with unique features derived from Hodgkin's disease. Int. J. Cancer. 1980;26:723-731
Schweizer A, Stahl P D, Rohrer J: A di-aromatic motif in the cytosolic tail of the mannose receptor mediates endosomal sorting. J. Biol. Chem. 2000;275:29694-29700.
Sheikh H, Yarwood H, Ashworth A, Isacke C M: Endol 80, an endocytic recycling glycoprotein related to the macrophage mannose receptor is expressed on fibroblasts, endothelial cells and macrophages and functions as a lectin receptor. J. Cell. Sci. 2000;113:1021-1032.
Skinnider B F, Mak T W: The role of cytokines in classical Hodgkin lymphoma. Blood. 2002;99:4283-4297.
Sorg U R, Morse T M, Patton W N, et al.: Hodgkin's cells express CD83, a dendritic cell lineage associated antigen. Pathology. 1997;29:294-299.
Steinman R M, Nussenzweig M C: Dendritic cells: features and functions. Immunol. Rev. 1980;53:127-147.
Strauchen J A: Interleukin receptors in lymphoid lesions. Relevance to diagnosis, biology, and therapy. Pathol. Annu. 1989;24 Pt 2:149-165.
Taylor M E, Conary J T, Lennartz M R, Stahl P D, Drickamer K: Primary structure of the mannose receptor contains multiple motifs resembling carbohydrate-recognition domains. J. Biol. Chem. 1990;265:12156-12162.
Uehira K, Amakawa R, Ito T, et al.: A Hodgkin's disease cell line, KM-H2, shows biphenotypic features of dendritic cells and B cells. Int. J. Hematol. 2001;73:236-244.
van den Berg A, Visser L, Poppema S: High expression of the CC chemokine TARC in Reed-Sternberg cells. A possible explanation for the characteristic T-cell infiltratein Hodgkin's lymphoma. Am J Pathol. 1999;154:1685-1691.
Wu K, Yuan J, Lasky L A: Characterization of a novel member of the macrophage mannose receptor type C lectin family. J. Biol. Chem. 1996;271:21323-21330.
Ziegler-Heitbrock H W, Thiel E, Futterer A, et al.: Establishment of a human cell line (Mono Mac 6) with characteristics of mature monocytes. Int. J. Cancer. 1988;41:456-461.

Zvaritch E, Lambeau G, Lazdunski M: Endocytic properties of the M-type 180-kDa receptor for secretory phospholipases A2. J. Biol. Chem. 1996;271:250-257.

TABLE 3

The DNA sequences of oligonucleotides primers
used in this study

| | |
|---|---|
| Primer | Sequence (5′ > 3′) |

| | | |
|---|---|---|
| 062 | GACCATGGAGCGGACATGATA | <400>23 |
| 063 | GGCTCTACCATCTGGGTTTGT | <400>24 |
| 078 | CCGCCATGTCGCGCGGCCT | <400>25 |
| 085 | ACCAAATCAGTCCGCCCATGAGAA | <400>26 |
| 086 | ATCATGTCCGCTCCATGGTCAGTA | <400>27 |
| 088 | TATTCAGAAGTTAAAAGCAGA | <400>28 |
| 090 | CCAAAAGGCCGTACTCCAAAA | <400>29 |
| 092 | GGAGGAAAACTGAATGACGCA | <400>30 |
| 094 | GAAAACGGTTGTGAAGATAAT | <400>31 |

1. A novel isolated nucleic acid molecule, comprising a DEC-205 intergenic splice variant or a derivative, homologue or analogue thereof.

2. The novel nucleic acid molecule according to claim 1 wherein said intergenic splice variant is a DEC-205/DCL-1 intergenic splice variant or a derivative, homologue or analogue thereof.

3. The nucleic acid molecule according to claim 2 comprising a nucleotide sequence encoding or a nucleotide sequence complementary to a nucleotide sequence encoding an amino acid sequence substantially as set forth in SEQ ID NO: 2 or SEQ ID NO: 21 or a derivative, homologue or mimetic thereof or having at least about 45% or greater similarity to at least 30 contiguous amino acids in SEQ ID NO: 2 or SEQ ID NO: 21 or a derivative, homologue or analogue of said nucleic acid molecule.

4. The nucleic acid molecule according to claim 2 in isolated form comprising a nucleotide sequence substantially as set forth in SEQ ID NO: 1 or SEQ ID) NO: 20 capable of hybridising to the sequence set forth in SEQ ID NO: 1 or SEQ ID NO: 20 under low stringency conditions at 42° C. or a derivative, homologue or analogue of said nucleic acid molecule.

5. The nucleic acid molecule of claim 4 wherein said nucleic acid molecule is a cDNA molecule.

6. The nucleic acid molecule according to claim 4 which encodes an amino acid sequence corresponding to an amino acid sequence set forth in SEQ ID NO: 2 or SEQ ID NO: 21 or a sequence having at least about 45% similarity to at least 30 contiguous amino acids in SEQ ID NO: 2 or SEQ ID NO: 21 or a derivative, homologue or analogue of said nucleic acid molecule.

7. The nucleic acid molecule according to claim 6 comprising a sequence of nucleotides substantially as set forth in SEQ ID NO: 1 or SEQ ID NO: 20.

8. The nucleic acid molecule according to claim 2 comprising a nucleotide sequence encoding or a nucleotide sequence complementary to a nucleotide sequence encoding an amino acid sequence substantially as set forth in SEQ ID NO: 5 or a derivative, homologue or mimetic thereof or having at least about 45% or greater similarity to at least 30 contiguous amino acids in SEQ ID NO: 5 or a derivative, homologue or analogue of said nucleic acid molecule.

9. The novel nucleic acid molecule according to claim 2 comprising a nucleotide sequence substantially as set forth in SEQ ID NO: 4 or a nucleotide sequence capable of hybridising to the sequence set forth in SEQ ID NO: 4 under low stringency conditions at 42° C. or a derivative, homologue or analogue of said nucleic acid molecule.

10. The nucleic acid molecule according to claim 9 wherein said nucleic acid molecule is a cDNA molecule.

11. The nucleic acid molecule according to claim 9 which encodes an amino acid sequence corresponding to an amino acid sequence set forth in SEQ ID NO: 5 or a sequence having at least 45% similarity to at least 30 contiguous amino acids in SEQ ID NO: 5 or a derivative, homologue or analogue of said nucleic acid molecule.

12. The novel nucleic acid molecule according to claim 2 comprising a nucleotide sequence substantially as set forth in SEQ ID NO: 32 or a nucleotide sequence capable of hybridising to the sequence set forth in SEQ ID NO: 32 under low stringency conditions at 42° C. or a derivative, homologue or analogue of said nucleic acid molecule.

13. The nucleic acid molecule according to claim 12 wherein said nucleic acid molecule is a genomic molecule.

14. The nucleic acid molecule according to claim 12 which encodes an amino acid sequence corresponding to an amino acid sequence set forth in SEQ ID NO: 5 or a sequence having at least about 45% similarity to at least 30 contiguous amino acids in SEQ ID NO: 5 or a derivative, homologue or analogue of said nucleic acid molecule.

15. The nucleic acid molecule according to claim 2 comprising a nucleotide sequence encoding or a nucleotide sequence complementary to a nucleotide sequence encoding an amino acid sequence substantially as set forth in SEQ ID NO: 8 or a derivative, homologue or mimetic thereof or having at least about 45% or greater similarity to at least 30 contiguous amino acids in SEQ ID NO: 8 or a derivative, homologue or analogue of said nucleic acid molecule.

16. The nucleic acid molecule according to claim 2 comprising a nucleotide sequence substantially as set forth in SEQ ID NO: 7 or a nucleotide sequence capable of hybridising to the sequence set forth in SEQ ID NO: 7 under low stringency conditions at 42° C. or a derivative, homologue or analogue of said nucleic acid molecule.

17. The nucleic acid molecule according to claim 16 wherein said nucleic acid molecule is a cDNA molecule.

18. The nucleic acid molecule according to claim 16 which encodes an amino acid sequence corresponding to an amino acid sequence set forth in SEQ ID NO: 8 or a sequence having at least about 45% similarity to at least 30 contiguous amino acids in SEQ ID NO: 8 or a derivative, homologue or analogue of said nucleic acid molecule.

19. The nucleic acid molecule according to claim 2 comprising a nucleotide sequence encoding or a nucleic acid molecule sequence complementary to a nucleotide sequence encoding an amino acid sequence substantially as set forth in SEQ ID NO: 11 or a derivative, homologue or mimetic thereof or having at least about 45% or greater similarity to at least 30 contiguous amino acids in SEQ ID NO: 11 or a derivative, homologue or analogue of said nucleic acid molecule.

20. The novel nucleic acid molecule according to claim 2 comprising a nucleotide sequence substantially as set forth in SEQ ID NO: 10 or a nucleotide sequence capable of hybridising to the sequence set forth in SEQ ID NO: 10 under low stringency conditions at 42° C. or a derivative, homologue or analogue of said nucleic acid molecule.

21. The nucleic acid molecule according to claim 20 wherein said nucleic acid molecule is a cDNA molecule.

22. The nucleic acid molecule according to claim 20 which encodes an amino acid sequence corresponding to an amino acid sequence set forth in SEQ ID NO: 11 or a sequence having at least about 45% similarity to at least 30 contiguous amino acids in SEQ ID NO: 11 or a derivative, homologue or analogue of said nucleic acid molecule.

23. The nucleic acid molecule according to claim 3 wherein said complementary nucleotide sequence is substantially as set forth in SEQ ID NO: 3 or 22 or capable of hybridising to the sequence set forth in SEQ ID NO: 3 or 22 under low stringency conditions at 42° C. or a derivative, homologue or analogue of said nucleic acid molecule.

24. The nucleic acid molecule according to claim 8 wherein said complementary nucleotide sequence is substantially as set forth in SEQ ID NO: 6 or capable of hybridising to the sequence set forth in SEQ ID NO: 6 under low stringency conditions at 42° C. or a derivative, homologue or analogue of said nucleic acid molecule.

25. The novel nucleic acid molecule according to claim 15 comprising a nucleotide sequence substantially as set forth in SEQ ID NO: 9 or a nucleotide sequence capable of hybridising to the sequence set forth in SEQ ID NO: 9 under low stringency conditions at 42° C. or a derivative, homologue or analogue of said nucleic acid molecule.

26. The novel nucleic acid molecule according to claim 19 comprising a nucleotide sequence substantially as set forth in SEQ ID NO: 12 or a nucleotide sequence capable of hybridising to the sequence set forth in SEQ ID NO: 12 under low stringency conditions at 42° C. or a derivative, homologue or analogue of said nucleic acid molecule.

27. The novel nucleic acid molecule according to claim 2 comprising a nucleotide sequence substantially as set forth in SEQ ID NO: 13 or a nucleotide sequence capable of hybridising to the sequence set forth in SEQ ID NO: 13 under low stringency conditions at 42° C. or a derivative, homologue or analogue of said nucleic acid molecule.

28. The nucleic acid molecule according to claim 27 wherein said nucleic acid molecule is a cDNA molecule.

29. An isolated protein comprising a DEC-205 intergenic splice variant or a derivative, homologue, analogue, chemical equivalent or mimetic thereof of said protein.

30. An isolated protein according to claim 29 wherein said intergenic splice variant is DEC-205/DCL-1 intergenic splice variant or a derivative, homologue, analogue, chemical equivalent or mimetic thereof of said protein.

31. The protein according to claim 30 having an amino acid sequence substantially as set forth in SEQ ID NO: 2 or SEQ ID NO: 21 or a sequence having at least about 45% similarity to at least 30 contiguous amino acids in SEQ ID NO: 2 or SEQ ID NO: 21 or a derivative, homologue, analogue, chemical equivalent or mimetic of said protein.

32. The protein according to claim 30 encoded by a nucleotide sequence substantially as set forth in SEQ ID NO: 1 or SEQ ID NO: 20 or capable of hybridising to the sequence set forth in SEQ ID NO: 1 or SEQ ID NO: 20 under low stringency conditions at 42° C. or a derivative, homologue, analogue, chemical equivalent or mimetic of said protein.

33. The protein according to claim 32 wherein said nucleotide sequence encodes an amino acid sequence substantially as set forth in SEQ ID NO: 2 or SEQ ID NO: 21 having at least about 45% similarity to at least 30 contiguous amino acids in SEQ ID NO: 2 or SEQ ID NO: 21 or a derivative, homologue, analogue, chemical equivalent or mimetic of said protein.

34. The protein according to claim 30 having an amino acid sequence substantially as set forth in SEQ ID NO: 5, SEQ ID NO: 8, or SEQ ID NO: 11 or a sequence having at least about 45% similarity to at least 30 contiguous amino acids in SEQ ID NO: 5, SEQ ID NO: 8, or SEQ ID NO: 11, respectively, or a derivative, homologue, analogue, chemical equivalent or mimetic of said protein.

35. The protein according to claim 30 encoded by a nucleotide sequence substantially as set forth in SEQ ID NOs: 4, 7 or 10 or capable of hybridising to the sequence set forth in SEQ ID NOs: 4, 7 or 10 under low stringency conditions at 42° C. or a derivative, homologue, analogue, chemical equivalent or mimetic of said protein.

36. The protein according to claim 35 wherein said nucleotide sequence encodes an amino acid sequence substantially as set forth in SEQ ID NOs: 5, 8 or 11 or an amino acid sequence having at least about 45% similarity to at least 30 contiguous amino acids in SEQ ID NOs: 5, 8 or 11 or a derivative, homologue, analogue, chemical equivalent or mimetic of said protein.

37. The protein according to claim 29 in a homodimeric form.

38. The protein according to claim 29 in a heterodimeric form.

39. A method of modulating DEC-205 SV expression or DEC-205 SV functional activity in a mammal, said method comprising administering to said mammal an agent for a time and under conditions sufficient to up-regulate, down-regulate or otherwise modulate expression of DEC-205 SV or functioning of DEC-205 SV.

40. A method for modulating DCL-1 expression or DCL-1 functional activity in a mammal, said method comprising administering to said mammal an agent for a time and under conditions sufficient to up-regulate, down-regulate or otherwise modulate said expression or functioning.

41. A method for regulating cellular activity in a subject said method comprising administering to said subject an effective amount of an agent for a time and under conditions sufficient to modulate DEC-205 SV expression or DEC-205 SV functional activity.

42. A method of regulating cellular activity in a subject said method comprising administering to said subject an effective amount of an agent for a time and conditions sufficient to modulate DCL-1 expression or DCL-1 functional activity.

43. The method according to claim 41, wherein said cellular activity is selected from the group consisting of: cellular endocytosis, late endosome targetting, intracellular signalling, Hodgkin and Reed-Sternberg cell functioning and antigen presenting cell antigen uptake.

44. A method for the treatment and/or prophylaxis of a condition characterized by aberrant, unwanted or otherwise inappropriate functioning of DEC-205 SV or DCL-1 in a subject, said method comprising administering to said subject an effective amount of an agent as hereinbefore defined for a time and under conditions sufficient to modulate the expression of DEC-205 SV or DCL-1 and/or functioning of DEC-205 SV or DCL-1.

45. A method for the treatment of Hodgkin's lymphoma in a mammal, said method comprising administering to said mammal an effective amount of a cytolytic and/or cytotoxic agent which agent interacts or otherwise associates with DEC-205 SV, for a time and under conditions sufficient for said agent to lyse, apoptose or otherwise kill Hodgkin and Reed-Sternberg cells.

46.-49. (canceled)

50. A pharmaceutical composition comprising at least one of DEC-205 SV, DCL-1, DEC-205 SV, DCL-1 or an agent capable of modulating DEC-205 SV or DCL-1 expression or DEC-205 SV or DCL-1 activity or a derivative, homologue, analogue, chemical equivalent or mimetic thereof together with one or more pharmaceutically acceptable carriers and/or diluents.

51. An isolated antibody directed to the protein according to claim 29.

52. An isolated antibody directed to the nucleic acid molecule according to claim 1.

53. The antibody according to claim 51 wherein said antibody is a monoclonal antibody.

54. The antibody according to claim 51 wherein said antibody is a polyclonal antibody.

55. A method of diagnosing or monitoring a mammalian disease condition, which disease condition is characterised by DEC-205 SV and/or DCL-1 expression, said method comprising screening for at least one of DEC-205 SV or DCL-1 or DEC-205 SV or DCL-1 in a biological sample isolated from said mammal.

56. A method for detecting an agent capable of modulating the function of DEC-205 SV or DCL-1 or its functional equivalent or derivative thereof said method comprising contacting a cell or extract thereof comprising said DEC-205 SV or DCL-1 or its functional equivalent or derivative with a putative agent and detecting an altered expression phenotype associated with said DEC-205 SV or DCL-1 or its functional equivalent or derivative.
